SEQUENCE LISTING (184 sequences)

SEQ ID NO: 1 - DNA, 522 bp, Zea mays
atgtctaggtctgtggagcctctcatagtcgggcgggtgattggagaagttctcgactcctttaacccatgtgtcaagatgatagtaacctacaactcaaacaaacttgtattcaatggccatgagatctacccatcagcaattgtatctaaacctagggtagaggttcaagggggtgatttgcggtctttcttcacattggttatgacagacccagatgttccaggaccaagtgatccatatctaagggagcaccttcattggatcgtgactgatatacctgggacaacagatgcctcctttgggcgagaggtcataagctatgagagcccaagacctaacatcggtatccacaggttcatttttgtgctcttcaagcagaagggtaggcaaactgtaaccgtgccatccttcagagatcatttcaacacccggcagtttgctgaggaaaatgaccttggcctcccagtagctgctgtctacttcaatgcacagagagaaactgcagctaggagacgttga

SEQ ID NO: 2 - protein, 173 aa, Zea mays
MSRSVEPLIVGRVIGEVLDSFNPCVKMIVTYNSNKLVFNGHEIYPSAIVSKPRVEVQGGDLRSFFTLVMTDPDVPGPSDPYLREHLHWIVTDIPGTTDASFGREVISYESPRPNIGIHRFIFVLFKQKGRQTVTVPSFRDHFNTRQFAEENDLGLPVAAVYFNAQRETAARRR

SEQ ID NO: 3 - DNA, 522 bp, Zea mays
atgtcaagggtgttggagcctctcattgtggggaaagtgattggtgaggtcctggaccatttcaaccccacggtgaagatggtggtcacctacaactccaacaagcaggtgttcaacgggcacgagttcttcccttcggcagtggccgccaagccgcgtgttgaggtccaagggggcgacctcaggtccttcttcacgttggtgatgaccgaccccgatgttcctggacctagtgatccatacttgagggagcaccttcactggattgtcactgatattcctgggactaccgatgcttcttttgggaaagaggtggtgagctacgagatcccaaagccaaacattggcatccacaggttcatctttgtgctgttccggcagaagagccggcaagcggtgaacccgcygtcgtcgaaggaccgcttcagcacccgccagttcgctgaggagaacgacctcggcctccccgtcgccgccgtctacttcaacgcgcagcgcgagaccgccgcccgccgacgctaa

SEQ ID NO: 4 - protein, 173 aa, Zea mays
  UNSURE (136)...(136): Xaa = any amino acid
MSRVLEPLIVGKVIGEVLDHFNPTVKMVVTYNSNKQVFNGHEFFPSAVAAKPRVEVQGGDLRSFFTLVMTDPDVPGPSDPYLREHLHWIVTDIPGTTDASFGKEVVSYEIPKPNIGIHRFIFVLFRQKSRQAVNPXSSKDRFSTRQFAEENDLGLPVAAVYFNAQRETAARRR

SEQ ID NO: 5 - DNA, 522 bp, Zea mays
atgtccaggtctgtggagcctctcatagtcgggcgggtgatcggagaagtcctcgactccttcaacccgtgtgtgaagatgatagtgacctacaactccaacaaactcgtgttcaatggccatgagatctacccatcagctgttgtgtccaaaccaagggtggcggttcaagggggcgatttgcggtctttcttcacattggttatgacagacccagatgttccaggaccaagtgatccatacctaagggagcaccttcattggatcgtgactgatatacctgggacaacagatgcctccttcgggcgacagatcataagctacgagagcccaagacctagcattggtatccacaggttcatttttgtgctcttcaagcagcagggtaggcaaaatgtaactgtgccatccttcagagatcatttcaacacccggcagttcgctgaggaaaatgaccttggcctccctgtagctgccgtctacttcaatgcacagagagaaactgctgctaggagacgctga

SEQ ID NO: 6 - protein, 173 aa, Zea mays
MSRSVEPLIVGRVIGEVLDSFNPCVKMIVTYNSNKLVFNGHEIYPSAVVSKPRVAVQGGDLRSFFTLVMTDPDVPGPSDPYLREHLHWIVTDIPGTTDASFGRQIISYESPRPSIGIHRFIFVLFKQQGRQNVTVPSFRDHFNTRQFAEENDLGLPVAAVYFNAQRETAARRR

SEQ ID NO: 7 - DNA, 531 bp, Zea mays
atgtctagagcgttggaacctctggtcgtcggcaaggtgatcggggaggtcatcgacaacttcaaccccacggtgaagatgacggttacctacggatccaacaagcaggtgttcaacggccatgagttctttccgtctgcggttctgtccaagccgcgcgtggaggttcagggcgacgacatgaggtccttcttcacgctggtcatgactgacccagatgtgccagggcctagtgatccatacctgagagagcacatccattggatcgtcaccgacattcctggaacaactgatgcttctttcggaagggagttggtgatgtacgagagcccgaagccgtacatcggcatccacaggttcgtcttcgtgctgttcaagcagagcagccggcagtcggcgcgcccgccctcgtccggcggcggcagggactacttcaacacccgccgctttgccgccgacaacaatcttggcctcccagttgccgcggtctacttcaacgcgcagcgggagactgccgcgcgccgccgctga

SEQ ID NO: 8 - protein, 176 aa, Zea mays
MSRALEPLVVGKVIGEVIDNFNPTVKMTVTYGSNKQVFNGHEFFPSAVLSKPRVEVQGDDMRSFFTLVMTDPDVPGPSDPYLREHIHWIVTDIPGTTDASFGRELVMYESPKPYIGIHRFVFVLFKQSSRQSARPPSSGGGRDYFNTRRFAADNNLGLPVAAVYFNAQRETAARRR

SEQ ID NO: 9 - DNA, 522 bp, Zea mays
atgtctagggcgttggagcctctagtcgtcggcaaggtgatcggcgaagtcatcgacaacttcaaccccacggtgaagatgacggtcacctacggctccgacaagcaggtgttcaacggccatgagttctttccgtcggcggttctgtccaagccgcgagtgcaggttcagggcgacgacatgaggtccttcttcacactggtcatgacggacccagatgtgccagggcctagtgatccatacctgagagagcacctccattggatggtcactgacattcctggaacaactgatgcttcttttggaagggagcaggtgatgtacgagagccccaaaccctacatcggcttccacaggttcgtcttcgtgctgttcaagcagagcagccgccagtcggtgtgcccgccctcgtccagggactacttcaacacccgccgctttgccgccgacaacaatcttggcctcccagtcgccgccgtctacttcaacgcgcagcgggagaccgccgcgcgccgccgctga

SEQ ID NO: 10 - protein, 173 aa, Zea mays
MSRALEPLVVGKVIGEVIDNFNPTVKMTVTYGSDKQVFNGHEFFPSAVLSKPRVQVQGDDMRSFFTLVMTDPDVPGPSDPYLREHLHWMVTDIPGTTDASFGREQVMYESPKPYIGFHRFVFVLFKQSSRQSVCPPSSRDYFNTRRFAADNNLGLPVAAVYFNAQRETAARRR

SEQ ID NO: 11 - DNA, 534 bp, Zea mays
atgtctagatctgtggagtctctcgtagtcggccgggtgatcggagaagttctcgactgcttcagcccatgtgtgaagatggtagtgacctacaactcaaacaggctcgtcttcaatggccacgagatctacccgtcagcagtcgtgtctaaaccaagagtagaggttcaagggggtgacttgcggtcgttcttcacattggttatgacagacccagacgtcccaggaccaagcgatccatatctaagggagcaccttcactggatcgtgactgatatacctgggacaactgatgcctcattcgggagagaagtcgtaagctatgagagcccgagacctggcattggtatccacaggttcatctttgttctcttcaagcagaagcgcaggcagcagcagactgtagcggcggtgccatcctccagcagggaccatttcatcacgcgtcagttcgctgcggaaaacgatcttggccaccctgtagccgctgtgtacttcaacgcccagagagaaactgctgctaggaggcgctga

SEQ ID NO: 12 - protein, 177 aa, Zea mays
MSRSVESLVVGRVIGEVLDCFSPCVKMVVTYNSNRLVFNGHEIYPSAVVSKPRVEVQGGDLRSFFTLVMTDPDVPGPSDPYLREHLHWIVTDIPGTTDASFGREVVSYESPRPGIGIHRFIFVLFKQKRRQQQTVAAVPSSSRDHFITRQFAAENDLGHPVAAVYFNAQRETAARRR

SEQ ID NO: 13 - DNA, 579 bp, Zea mays
atgctcaggctgcagcttcctcagtcccatagggttatttttctgcagtatttgtcagcaaccgatcctttggttatggctcgtgtcctacaggatgtgttggatacctttacaccaaccattccactaagaataacatacaacaatagtcaagttctggcaggtgctgagctaaagccatctgcggttataaataaaccacgagtcgatatcggtggcaatgacatgaggactttctacaccctggtactgattgacccggacgccccaagtccaagccatccatcactaagggagtacttgcactggatgatgacagatattcctgaaacaactagtgtcaacttcggccaagagctagtattttatgagagaccagatccaagatctggtatccacaggctggtatttgtgttgttccgccaacttggcaggggtacggtttttgcaccagaaatgcgccaaaacttcaactgcagaagctttgcacggcaatatcacctcagcattgccagtgctacacatttcaactgtcaaagggaaggtggatcgggtggaagaaggtttagggaagagtag

SEQ ID NO: 14 - protein, 192 aa, Zea mays
MLRLQLPQSHRVIFLQYLSATDPLVMARVLQDVLDTFTPTIPLRITYNNSQVLAGAELKPSAVINKPRVDIGGNDMRTFYTLVLIDPDAPSPSHPSLREYLHWMMTDIPETTSVNFGQELVFYERPDPRSGIHRLVFVLFRQLGRGTVFAPEMRQNFNCRSFARQYHLSIASATHFNCQREGGSGGRRFREE

SEQ ID NO: 15 - DNA, 528 bp, Zea mays
atgtcagcaaccgatcatttggttatggctcgtgtcatacaggatgtattggatccctttacaccaaccattccactaagaataacgtacaacaataggctacttctgccaagtgctgagctaaagccatccgcggttgtaagtaaaccacgagtcgatatcggtggcagtgacatgagggctttctacaccctggtactgattgacccggatgccccaagtccaagccatccatcactaagggagtacttgcactggatggtgacagatattccagaaacaactagtgtcaactttggccaagagctaatattttatgagaggccggacccaagatctggcatccacaggctggtatttgtgctgttccgtcaacttggcagggggacagtttttgcaccagaaatgcgccacaacttcaactgcagaagctttgcacggcaatatcacctcagcattgccaccgctacacatttcaactgtcaaagggaaggtggatccggcggaagaaggtttagggaagagtag

SEQ ID NO: 16 - protein, 175 aa, Zea mays
MSATDHLVMARVIQDVLDPFTPTIPLRITYNNRLLLPSAELKPSAVVSKPRVDIGGSDMRAFYTLVLIDPDAPSPSHPSLREYLHWMVTDIPETTSVNFGQELIFYERPDPRSGIHRLVFVLFRQLGRGTVFAPEMRHNFNCRSFARQYHLSIATATHFNCQREGGSGGRRFREE

SEQ ID NO: 17 - DNA, 519 bp, Zea mays
atggcgcgcttcgtggatccgctggtggtggggcgggtgatcggcgaggtggtggacctgttcgtgccttccatctccatgaccgtcgcctatgatggccccaaggacatcagcaacggctgcctcctcaagccgtccgccaccgccgcgccgccgctcgtccgcatctccggccgccgcaacgacctctacacgctgatcatgacggaccccgatgcgcctagccccagcaacccgaccatgagggagtacctccactggatagtgattaacataccaggaggaacagatgctactaaaggtgaggaggtggtggagtacatgggcccgcggccgccggtgggtatccaccgctacgtgctggtgctgttcgagcagaagacgcgcgtgcacgcggaggcccccggcgaccgcgccaacttcaagacgcgcgcgttcgcggcggcgcacgagctcggcctccccactgccgtcgtctacttcaacgcgcagaaggagcccgccagccgccgccgctag

SEQ ID NO: 18 - protein, 172 aa, Zea mays
MARFVDPLVVGRVIGEVVDLFVPSISMTVAYDGPKDISNGCLLKPSATAAPPLVRISGRRNDLYTLIMTDPDAPSPSNPTMREYLHWIVINIPGGTDATKGEEVVEYMGPRPPVGIHRYVLVLFEQKTRVHAEAPGDRANFKTRAFAAAHELGLPTAVVYFNAQKEPASRRR

SEQ ID NO: 19 - DNA, 513 bp, Zea mays
atggcgcggttcgtggacccgctggtggtggggcgggtgatcggcgaggtggtggacctgttcgtgccctccgtctccatgaccgtcgcctatggccccaaagacatcagcaacggctgcctcctcaagccgtccgccaccgccgcgccgccgctcgtccgcatctccggccgccgcgacgacctctacacgctgatcatgacggacccagatgcgcctagccccagcgacccgaccatgagggagtacctccactggatagtgactaacataccaggaggaacggatgcaaacaaagaggtggtggagtacatgggcccgcggccgccggtcggaatccaccgctacgtgctggtgctgttcgagcagaagacgcgtgtgcacgcggagggtcccggtgagcgcgccaacttcaacacacgcgcgttcgcggcggcgcacgagctcggcctccccaccgccgtcgtgtacttcaacgcgcagaaagagccggccaaccaccgccgccgctag

SEQ ID NO: 20 - protein, 170 aa, Zea mays
MARFVDPLVVGRVIGEVVDLFVPSVSMTVAYGPKDISNGCLLKPSATAAPPLVRISGRRDDLYTLIMTDPDAPSPSDPTMREYLHWIVTNIPGGTDANKEVVEYMGPRPPVGIHRYVLVLFEQKTRVHAEGPGERANFNTRAFAAAHELGLPTAVVYFNAQKEPANHRRR

SEQ ID NO: 21 - DNA, 543 bp, Zea mays
atggctgcccatgtggacccgctggttgtggggagggtgatcggcgacgtggtggacttgttcgtgccgacggtggccgtgtcggcgcgcttcggcgccaaggacctcaccaacggctgcgagatcaagccatccgtcgccgcggccgctcccgccgtcctcatcgccggcagggccaacgacctcttcaccctggttatgactgacccagatgctccgagccctagcgagccaacgatgagggagttgctccactggctggtggttaacataccaggtggagcagatgcttctcaaggcggtgagacggtggtgccgtacgtgggcccgcgcccgccggtgggtatccaccgctacgtgctggtggtgtaccagcagaaggcccgcgtcacggctccgccgtcgctggcgccggcgacggaggcgacgcgcgcacggttcagcaaccgcgccttcgccgaccgccatgacctaggcctccctgtcgccgccatgttcttcaacgcgcagaaggagacagctagtcgccgccgccactactga

SEQ ID NO: 22 - protein, 180 aa, Zea mays
MAAHVDPLVVGRVIGDVVDLFVPTVAVSARFGAKDLTNGCEIKPSVAAAAPAVLIAGRANDLFTLVMTDPDAPSPSEPTMRELLHWLVVNIPGGADASQGGETVVPYVGPRPPVGIHRYVLVVYQQKARVTAPPSLAPATEATRARFSNRAFADRHDLGLPVAAMFFNAQKETASRRRHY

SEQ ID NO: 23 - DNA, 534 bp, Zea mays
atgtctgatgtggagccgctggttctggctcatgtcatacgagatgtgttggattcatttgcaccaagtatcgggctcagaataacctacaacagcaggttacttctatcaggtgttgagctgaaaccatccgcggttgtgaataagccaagagttgatgttgggggcaccgacctcagggtgttctacacattggtattagtggatccagatgccccaagcccaagcaatccatcactgagggagtatctgcactggatggtgatagacattcctggaacaactggagccagctttggtcaggagctcatgttttacgagaggccagagccgaggtccggcatacaccgcatggtgttcgtgctgttccggcagctcggcagggggacggtgtttgcaccagacatgcggcacaacttcaactgcaagagcttcgcccgtcagtaccacctggacgtcgtggctgccacgtatttcaactgccaaagggaggcaggatccgggggcagaaggttcaggccggagagctcgtaa

SEQ ID NO: 24 - protein, 177 aa, Zea mays
MSDVEPLVLAHVIRDVLDSFAPSIGLRITYNSRLLLSGVELKPSAVVNKPRVDVGGTDLRVFYTLVLVDPDAPSPSNPSLREYLHWMVIDIPGTTGASFGQELMFYERPEPRSGIHRMVFVLFRQLGRGTVFAPDMRHNFNCKSFARQYHLDVVAATYFNCQREAGSGGRRFRPESS

SEQ ID NO: 25 - DNA, 555 bp, Zea mays
atggccaacgattccttggtcacagctcgtgtcataggagatgtcctggaccccttctacagctccattgatctgatggtgctgttcaacggtttgcctattgttagtggcgtggagctgcgtcctcccgcggtctccgagagacccagggtcgagatcggaggagatgattatcgcgttgcatgtactctggtgatggtcgatccagatgccccgaacccaagcaacccgaccctgagggagtacctgcactggatggtgactgacatcccagcgtccaccgatgatacacacggtcgggaggtgatgtgctacgaggcccctaatccgacgacgggcatccaccgcatggtgctggtgctgttccggcagctggggcgggagacggtgtacgcgccatccaggcgccacaacttcagcacgcgcgccttcgcccgccgctacaacctcggcgcgcccgtcgcagccatgtacttcaactgccagcgccagaacggctccggcggacggaggttcaccgggccctacaccggcggcagacgtggtggtgcttga

SEQ ID NO: 26 - protein, 184 aa, Zea mays
MANDSLVTARVIGDVLDPFYSSIDLMVLFNGLPIVSGVELRPPAVSERPRVEIGGDDYRVACTLVMVDPDAPNPSNPTLREYLHWMVTDIPASTDDTHGREVMCYEAPNPTTGIHRMVLVLFRQLGRETVYAPSRRHNFSTRAFARRYNLGAPVAAMYFNCQRQNGSGGRRFTGPYTGGRRGGA

SEQ ID NO: 27 - DNA, 522 bp, Zea mays
atgcagcgtggggatccgctggtggtgggccgcatcatcggcgacgtggtggaccccttcgtgcgccgggtgccgctccgcgtcgcctacgccgcgcgcgaggtctccaacggctgcgagctcaggccctccgccatcgccgaccagccgcgcgtcgaggtcggcggacccgacatgcgcaccttctacaccctcgtgatggtagatcctgatgcgccgagccccagcgatcccaacctcagggagtacctgcactggctggtcactgatattccggcgacgactggagtatcttttgggaccgaggtcgtgtgctacgagagcccacggccggtgctggggatccaccgggtcgtgtttctgctcttccagcagctcggccggcagacggtgtacgccccggggtggcggcagaacttcagcacccgcgacttcgccgagctctacaacctcggcttgccggtcgccgccgtctacttcaactgccagagggagtccggaaccggtgggagaagaatgtga

SEQ ID NO: 28 - protein, 173 aa, Zea mays
MQRGDPLVVGRIIGDVVDPFVRRVPLRVAYAAREVSNGCELRPSAIADQPRVEVGGPDMRTFYTLVMVDPDAPSPSDPNLREYLHWLVTDIPATTGVSFGTEVVCYESPRPVLGIHRVVFLLFQQLGRQTVYAPGWRQNFSTRDFAELYNLGLPVAAVYFNCQRESGTGGRRM

SEQ ID NO: 29 - DNA, 534 bp, Zea mays
atggccggcagggacagggagccgctggtggttggtagggtggtcggcgacgtgctggaccccttcgtccggaccaccaacctcagggtcagctacggggccaggaccgtgtccaacggctgcgagctcaagccgtccatggtggtgcaccagcccagggtcgaggtcgggggacctgacatgaggaccttctacaccctcgtgatggtggacccggatgctccgagcccaagcgacccgaaccttagggagtacctacactggctggtgacggatattccgggaactactggggcagcatttgggcaagaggtgatctgctacgagagccctcggccgaccatggggatccaccgcttcgtgctggtgctgttccagcagctggggcggcagacggtgtacgccccgggctggcgccagaacttcaacaccagggacttcgccgagctctacaacctgggcccgcccgtcgccgccgtctacttcaactgccagcgtgaggccggctctgggggcaggaggatgtactcgtga

SEQ ID NO: 30 - protein, 177 aa, Zea mays
MAGRDREPLVVGRVVGDVLDPFVRTTNLRVSYGARTVSNGCELKPSMVVHQPRVEVGGPDMRTFYTLVMVDPDAPSPSDPNLREYLHWLVTDIPGTTGAAFGQEVICYESPRPTMGIHRFVLVLFQQLGRQTVYAPGWRQNFNTRDFAELYNLGPPVAAVYFNCQREAGSGGRRMYS

SEQ ID NO: 31 - DNA, 525 bp, Zea mays
atgtcaagggacccacttgtcgtaggcaacgtagttggagatatcttggacccatttatcaaatcagcatcactcagagtcctatacaacaatagagaactgactaatggatctgagctcaggccatcgcaagtagcttatgaaccaaggattgagattgctggatatgacatgaggaccctttacactttggtaatggtggatcctgactcaccaagtccaagcaatccaacaaaaagagagtaccttcactggttggtgacagatattccagaatcaacagatgtgagctttggaaatgaggtagtaagctatgaaagcccaaagccaagtgctggaatacatcgcttcgtctttgttctgttccgccaatctgtcaggcaaactatttatgcgccaggatggagacaaaatttcaacacaagagacttctcagcactctataatctaggaccacctgtggcctcagtgttcttcaactgccaaagggagaatgggtgcggtggcagacgatatattagatga

SEQ ID NO: 32 - protein, 174 aa, Zea mays
MSRDPLVVGNVVGDILDPFIKSASLRVLYNNRELTNGSELRPSQVAYEPRIEIAGYDMRTLYTLVMVDPDSPSPSNPTKREYLHWLVTDIPESTDVSFGNEVVSYESPKPSAGIHRFVFVLFRQSVRQTIYAPGWRQNFNTRDFSALYNLGPPVASVFFNCQRENGCGGRRYIR

SEQ ID NO: 33 - DNA, 540 bp, Zea mays
atgttcaatatgtctagggacccattggtcgtcggcaatgttgtgggagatattgtggatcccttcatcacaacggcgtcactgagagtgttctacaacaataaggagatgacaaatggttctgatcttaagccatctcaagtgatgaatgaaccaagggtccacgtcggtgggcgtgacatgaggactctttacacacttgtaagtgtcatggtggacccagatgcaccaagccccagtaaccctacaaaaagagagaaccttcactggttggtgacagacattccagagacaactgatgccagtttcgggaacgaaatagttccgtacgagagcccacgtccaatcgccggaatccatcgcttcgcattcgtcctgttcaggcagtcagtgaggcagaccacctatgcgccgggatggagatcaaacttcaacactagagacttcgcagccatctacggccttggctcccctgtcgctgcagtgtacttcaactgccagagagagaacggatgtggtggaagaaggtacataaggtga

SEQ ID NO: 34 - protein, 179 aa, Zea mays
MFNMSRDPLVVGNVVGDIVDPFITTASLRVFYNNKEMTNGSDLKPSQVMNEPRVHVGGRDMRTLYTLVSVMVDPDAPSPSNPTKRENLHWLVTDIPETTDASFGNEIVPYESPRPIAGIHRFAFVLFRQSVRQTTYAPGWRSNFNTRDFAAIYGLGSPVAAVYFNCQRENGCGGRRYIR

SEQ ID NO: 35 - DNA, 222 bp, Zea mays
ggcaatgaaatagttccctatgaaagcccaaggccaccagctggaattcatcgaattgtttttgtgctgttcaaacagcaaacaagacaaacagtttatgcaccaggatggcggcaaaatttcaacatcagagacttctcggcaatttacaatcttggagcaccagttgctgcattatacttcaactgccaaaaggaaagtggtgttggtggcagaaggtag

SEQ ID NO: 36 - protein, 73 aa, Zea mays
GNEIVPYESPRPPAGIHRIVFVLFKQQTRQTVYAPGWRQNFNIRDFSAIYNLGAPVAALYFNCQKESGVGGRR

SEQ ID NO: 37 - DNA, 195 bp, Zea mays
atgtcaagggatccactagtggtaggacacgtggtgggtgacattttggacccgtttactaaagcagcctcgcttaaggttctgtacaacaacaaggaactgaccaatgggtctgagctcaagccatcgcaggtagcaaatgaaccgagggttgaaataattggtgggcgcgacatgagcaacctttacactctg

SEQ ID NO: 38 - protein, 65 aa, Zea mays
MSRDPLVVGHVVGDILDPFTKAASLKVLYNNKELTNGSELKPSQVANEPRVEIIGGRDMSNLYTL

SEQ ID NO: 39 - DNA, 528 bp, Zea mays
atgtccagggatccgcttgtggtgggaagcatcgtgggcgacgtcgtggactacttctcggcgtcggcgctgctccgagtgatgtacggcgggcgcgagatgacctgcgggtcggagctcaggccgtcgcaggtggcgagcgagccgacggtgcacatcacggggggccgcgacgggaggccggtgctctacacactggtgatgctggaccccgatgcgcccagcccaagcaacccctccaagcgggagtatctccattggttggtgactgacataccagaaggagctggtgccaatcatgggaacgaggtggtggcgtacgagagcccccggccatcggcggggatccaccgattcgtgttcatcgtgttccggcaggcggtccggcaggcgatctacgcgcctgggtggcgcgccaacttcaacaccagggacttcgccgcctgctacagcctcggaccgcctgtcgccgccacctacttcaactgccagagggagggcggctgcggtggtcggaggtacaggtga

SEQ ID NO: 40 - protein, 175 aa, Zea mays
MSRDPLVVGSIVGDVVDYFSASALLRVMYGGREMTCGSELRPSQVASEPTVHITGGRDGRPVLYTLVMLDPDAPSPSNPSKREYLHWLVTDIPEGAGANHGNEVVAYESPRPSAGIHRFVFIVFRQAVRQAIYAPGWRANFNTRDFAACYSLGPPVAATYFNCQREGGCGGRRYR

SEQ ID NO: 41 - DNA, 303 bp, Zea mays
atggcgccggcggctaacgattccttggtcacagctcatgtgataggagatgtcctggaccccttctacacagccgttgacatgatgatcctgttcggtggtgctcccatcatcagcggcatggagctgcgcgctcaggcagtctctgataggccaagggttgagatcggaggagaagattatcgagatgcatataccctggtgatggtcgatcctgatgctcctaacccaagcaacccaaccttgagggagtacttgcactggatggtgactgacatccccgcatcaactgacaatacacac

SEQ ID NO: 42 - protein, 101 aa, Zea mays
MAPAANDSLVTAHVIGDVLDPFYTAVDMMILFGGAPIISGMELRAQAVSDRPRVEIGGEDYRDAYTLVMVDPDAPNPSNPTLREYLHWMVTDIPASTDNTH

SEQ ID NO: 43 - DNA, 258 bp, Zea mays
cgtgagatgatgtgctacgagcccccagccccgtcgacgggcatccaccggatggtgctggtgctgttccagcagcttgggcgggacacggtgttcgcggcgccgtcgaggcgccacaacttcagcacccgtggcttcgcccgccgctacaacctcggcgcgcccgtcgccgccatgtacttcaactgccagcgccagaccggctccggcggccccaggttcaccgggccctacaccagccgccgtcgtgcgggctga

SEQ ID NO: 44 - protein, 85 aa, Zea mays
REMMCYEPPAPSTGIHRMVLVLFQQLGRDTVFAAPSRRHNFSTRGFARRYNLGAPVAAMYFNCQRQTGSGGPRFTGPYTSRRRAG

SEQ ID NO: 45 - DNA, 315 bp, Zea mays
cgcgaggtgatctgctacgagagccctcggccgccggcggggatccaccgcgtggtgttcgtgctctaccagcagacggcgcgcggcgccgtcgaccagccgccgcttctccgccacaacttctgcacccgcagcttcgccgtcgaccacgggctgggcgcccccgtcgccgccgccttcttcacctgtcagcccgagggtggcaccggcggccgccgccacgtcctccgccagccagcaaggtcaccagcgcctatagatgtccaaacagtacgggccgtccgtttggcccgtgacccggcacgattttggccc

SEQ ID NO: 46 - protein, 105 aa, Zea mays
REVICYESPRPPAGIHRVVFVLYQQTARGAVDQPPLLRHNFCTRSFAVDHGLGAPVAAAFFTCQPEGGTGGRRHVLRQPARSPAPIDVQTVRAVRLARDPARFWP

SEQ ID NO: 47 - DNA, 588 bp, Zea mays
attttgaggagaccggtggttgcaagattatattcaactttaagcaaaagtatggaaaaacacggtgttgtgcccgatgttatcgatgttgcccccgaacaacaagtggaagtgtcgtatcctagtggtgtaaaggtagactttggtaacgaattaactccaacacaagtcaaagatatcccggcagtaaaatggccggccgataaagattccctttacacactttgcatgaccgatcctgatgccccaagtcgaaaagaacccaagttccgtgaatggcaccattggctcgttggaaatatcccaggaggagaggtctcaaaaggcgaagttctttctgaatatgttgggtctggaccaccaccaaatacaggtcttcataggtatgttttcttggtgtacaaacagaatggtaaattgaattttgatgaaccaagattgaccaatcgatccggggataatagaggtggattttctattagaaagtttgcagcaaaatataatcttgggcaacctgttgctggcaatttgtaccaagctgagtatgatgattatgttccaattttgtacaagcaattgggaggttaa

SEQ ID NO: 48 - protein, 195 aa, Zea mays
ILRRPVVARLYSTLSKSMEKHGVVPDVIDVAPEQQVEVSYPSGVKVDFGNELTPTQVKDIPAVKWPADKDSLYTLCMTDPDAPSRKEPKFREWHHWLVGNIPGGEVSKGEVLSEYVGSGPPPNTGLHRYVFLVYKQNGKLNFDEPRLTNRSGDNRGGFSIRKFAAKYNLGQPVAGNLYQAEYDDYVPILYKQLGG

SEQ ID NO: 49 - DNA, 333 bp, Zea mays
gtgatggtggatccagactccccaagtccaagtaacccaacaaaaagagaataccttcattggttggtgacagacatcccggaatcagcaaatgctagctatggaaacgaaatcgtcagctatgaaaacccaaagccaactgctggaatacatcgctttgtctttgttctcttccgccagtctgtccagcaaaccgtttatgcaccaggatggagacaaaatttcaacacgagagacttttctgcgttctataatcttggacctcctgtggctgcagtgttcttcaattgtcaaagggagaatgggtgtggaggcagacgatatattagataa

SEQ ID NO: 50 - protein, 110 aa, Zea mays
VMVDPDSPSPSNPTKREYLHWLVTDIPESANASYGNEIVSYENPKPTAGIHRFVFVLFRQSVQQTVYAPGWRQNFNTRDFSAFYNLGPPVAAVFFNCQRENGCGGRRYIR

SEQ ID NO: 51 - DNA, 267 bp, Zea mays
cgagagctcataccatatgagagcccaagccccaccatgggcatccaccgtcttgtgttggtgctctaccagcaattggggcggggcacggtgtttgcgccgcaagttcgtcagaacttcaacttgcgtaatttcgcacgccgtttcaacctcggcaagcctgtggccgcgacgtacttcaactgtcagcggcaaacaggcacaggtgggagaaggttcacttgtgtttttgatcatgtcgttcaaggtgaaggccggcaagcttga

SEQ ID NO: 52 - protein, 88 aa, Zea mays
RELIPYESPSPTMGIHRLVLVLYQQLGRGTVFAPQVRQNFNLRNFARRFNLGKPVAATYFNCQRQTGTGGRRFTCVFDHVVQGEGRQA

SEQ ID NO: 53 - DNA, 228 bp, Zea mays
aatgaaattgtcagctatgaaaacccaaagccatctgctggaatacatcgctttgtctttgtactcttccgccagtctgtacagcaaaccgtttatgcaccaggatggagacaaaatttcaacacgagagacttttctgcgctctataatcttggacctccagtggctgcagttttcttcaattgtcaaagggagaatgggtgtggaggaagacgatatattagataa

SEQ ID NO: 54 - protein, 75 aa, Zea mays
NEIVSYENPKPSAGIHRFVFVLFRQSVQQTVYAPGWRQNFNTRDFSALYNLGPPVAAVFFNCQRENGCGGRRYIR

SEQ ID NO: 55 - DNA, 192 bp, Zea mays
atggctaatgactccttgacgaggggccacataatcggggatgtcttagacccgtttactagctcagtgtctctaagtgtcctgtatgatggcagaccagtgtttgatgggatggagtttcgggcgtcggcggtgtcggtgaaacctagagttgagattggaggtgatgattttcgagtggcctatacccta

SEQ ID NO: 56 - protein, 64 aa, Zea mays
MANDSLTRGHIIGDVLDPFTSSVSLSVLYDGRPVFDGMEFRASAVSVKPRVEIGGDDFRVAYTL

SEQ ID NO: 57 - DNA, 540 bp, Oryza sativa
atggccggaagtggcagggacagggaccctcttgtggttggtagggttgtgggtgatgtgctggacgcgttcgtccggagcaccaacctcaaggtcacctatggctccaagaccgtgtccaatggctgcgagctcaagccgtccatggtcacccaccagcctagggtcgaggtcggcggcaatgacatgaggacattctacacccttgtgatggtagacccagatgcaccaagcccaagtgaccctaaccttagggagtatctacattggttggtcactgatattcctggtactactgcagcgtcatttgggcaagaggtgatgtgctacgagagcccaaggccaaccatggggatccaccggctggtgttcgtgctgttccagcagctggggcgtcagacagtgtacgcgcccgggtggcgtcagaacttcaacaccaaggacttcgccgagctctacaacctcggctcgccggtcgccgccgtctacttcaactgccagcgcgaggcaggctccggcggcaggagggtctacccctag

SEQ ID NO: 58 - protein, 179 aa, Oryza sativa
MAGSGRDRDPLVVGRVVGDVLDAFVRSTNLKVTYGSKTVSNGCELKPSMVTHQPRVEVGGNDMRTFYTLVMVDPDAPSPSDPNLREYLHWLVTDIPGTTAASFGQEVMCYESPRPTMGIHRLVFVLFQQLGRQTVYAPGWRQNFNTKDFAELYNLGSPVAAVYFNCQREAGSGGRRVYP

SEQ ID NO: 59 - DNA, 534 bp, Oryza sativa
atgagcatgtcgagggacccgctggtggtggggagcatcgtcggcgacgtggtggaccacttcggcgcgtcggcgctgctgaggctgttctacaaccaccgcgagatgacgagcgggtcggagctcaggccgtcgcaggtcgccggcgagccggccgtccagatcaccggaggccgcgatgggagggcgctctacacgctcgtaatggtggaccctgatgcacctagccccagcaacccttccaaaagggaataccttcattggttggtaactgacgtaccagaaggaggcgatacgagtaaagggacggaggtggtggcgtacgagagcccgcggccgacagcggggatccaccggttggtgttcatcgtgttccggcagacagtgcggcagtccatctacgcgccggggtggcgctccaacttcaacaccagggacttcgccgcctgctacagcctcggctcccccgtcgccgccgcctacttcaactgccagagggagggcggctgcggcggccggaggtacaggtcatga

SEQ ID NO: 60 - protein, 177 aa, Oryza sativa
MSMSRDPLVVGSIVGDVVDHFGASALLRLFYNHREMTSGSELRPSQVAGEPAVQITGGRDGRALYTLVMVDPDAPSPSNPSKREYLHWLVTDVPEGGDTSKGTEVVAYESPRPTAGIHRLVFIVFRQTVRQSIYAPGWRSNFNTRDFAACYSLGSPVAAAYFNCQREGGCGGRRYRS

SEQ ID NO: 61 - DNA, 522 bp, Oryza sativa
atgtcacgaggtagggatcctttggcattgagccaggtaattggcgatgtgttggatcctttcataaagtcagctgcaatgaggattaattatggtgagaaggagattacaaatggaactggagtacgatcatctgctgttttcactgcaccacatgttgagattgaaggtcgtgaccaaacgaagctctacacacttgttatggtggatcctgatgcgccaagtccaagcaaaccagaatacagggaatatttgcattggttggtgacagacatcccagaggcaatagatgcacgttttggcaatgaaatagttccgtacgaagctccacggccaccggctggaattcatcggcttgtttttgtgctattcaaacaggaagcacgacaaacagtttatgctccaggatggcggcaaaatttcaacgtcagagatttctctgcattttacaatcttggaccacctgttgctgcattatacttcaactgccagaaggagagtggtgttggtggcagaaggtag

SEQ ID NO: 62 - protein, 173 aa, Oryza sativa
MSRGRDPLALSQVIGDVLDPFIKSAAMRINYGEKEITNGTGVRSSAVFTAPHVEIEGRDQTKLYTLVMVDPDAPSPSKPEYREYLHWLVTDIPEAIDARFGNEIVPYEAPRPPAGIHRLVFVLFKQEARQTVYAPGWRQNFNVRDFSAFYNLGPPVAALYFNCQKESGVGGRR

SEQ ID NO: 63 - DNA, 522 bp, Oryza sativa
atgtctagggtgctggagcctctcgtcgtcgggaaggtgatcggagaggtcatcgacaacttcaaccccacggtgaagatgacggcgacctacagctccaacaagcaggtgttcaacggccacgagttattcccgtcggcggtcgtgtccaagccgcgagtcgaggttcagggcggcgacctgaggtctttcttcacactggttatgacagatccagacgtgccagggcctagtgatccgtacctgagggagcacctccactggatcgtcactgatattcctggcaccactgatgcttcctttgggagggaggtggtgagctacgagagcccgaagcccaacattggcatccacaggttcgtcctcgtgctgttcaagcagaagcgccgtcaggcggtgaccccgccatcctccagggactacttcagcacccgccgcttcgccgccgacaacgacctcggcctccccgtcgccgccgtctacttcaacgcgcagcgagagacggccgctcgccgccgctaa

SEQ ID NO: 64 - protein, 173 aa, Oryza sativa
MSRVLEPLVVGKVIGEVIDNFNPTVKMTATYSSNKQVFNGHELFPSAVVSKPRVEVQGGDLRSFFTLVMTDPDVPGPSDPYLREHLHWIVTDIPGTTDASFGREVVSYESPKPNIGIHRFVLVLFKQKRRQAVTPPSSRDYFSTRRFAADNDLGLPVAAVYFNAQRETAARRR

SEQ ID NO: 65 - DNA, 522 bp, Oryza sativa; identical to SEQ ID NO: 63

SEQ ID NO: 66 - protein, 173 aa, Oryza sativa; identical to SEQ ID NO: 64

SEQ ID NO: 67 - DNA, 525 bp, Oryza sativa
atgtcaagggacccacttgtcgtaggacatgttgttggggatatcttagacccattcaacaaatcagcatcactcaaggtcctatacaacaacaaggaattaacaaatgggtctgagctcaaaccgtcacaggtagcaaatgaaccaaggattgaaattgctggccgcgacataaggaacctttacactctggtgatggtggatcctgactcgccaagtccaagcaacccaacaaaaagagaataccttcattggttggtgacagacattccagaatcggcaaatgctagttatggaaatgaagttgtcagttatgaaagcccaaaaccaactgcagggatacatcgttttgtctttatattatttcgccaatatgtacaacagactatttatgcaccaggatggagaccaaatttcaatacaagagatttttccgcactgtataatcttggacctcctgtggcagcagtgttcttcaattgccagagggagaacggatgtggaggcagacggtacattagataa

SEQ ID NO: 68 - protein, 174 aa, Oryza sativa
MSRDPLVVGHVVGDILDPFNKSASLKVLYNNKELTNGSELKPSQVANEPRIEIAGRDIRNLYTLVMVDPDSPSPSNPTKREYLHWLVTDIPESANASYGNEVVSYESPKPTAGIHRFVFILFRQYVQQTIYAPGWRPNFNTRDFSALYNLGPPVAAVFFNCQRENGCGGRRYIR

SEQ ID NO: 69 - DNA, 543 bp, Oryza sativa
atgtcgtcggcgaacagcctggtgctggggcgggtgatcggcgacgtggtggacctgttctcgccggaggtgacgctccgggtgatgtacaacggcgtgcgggtcgtcaacggcgaggacctccggccgtcggcggtgtcggcgaggcccagcgtcgaggtcggaggggatctccaccagttctacacgatcgtgatggtggatccagatgctccaaacccaagcaatccgacgttgagagagtacttacactggttggtgacagatattcctggaacaactgatgcgaactatgggcgcgaggtggtgtgctacgagagcccccggccagcggcggggatccaccgggtggcggtggtgctgttccggcagatggcgcgcggcggcgtggaccagccgccgctgctccgccacaacttctccacccgcggcttcgccgacgaccacgccctcggcgcccccgtcgccgccgccttcttcacctgcaagcccgagggcggcaccggcggccgccgcttccggccaccgtcacggcatagctag

SEQ ID NO: 70 - protein, 180 aa, Oryza sativa
MSSANSLVLGRVIGDVVDLFSPEVTLRVMYNGVRVVNGEDLRPSAVSARPSVEVGGDLHQFYTIVMVDPDAPNPSNPTLREYLHWLVTDIPGTTDANYGREVVCYESPRPAAGIHRVAVVLFRQMARGGVDQPPLLRHNFSTRGFADDHALGAPVAAAFFTCKPEGGTGGRRFRPPSRHS

SEQ ID NO: 71 - DNA, 522 bp, Oryza sativa
atgtcgagggtgctggagcctctcattgtggggaaggtgatcggcgaggtgctggacaacttcaaccccacggtgaagatgacggccacctacggcgccaacaagcaggtgttcaacggccacgagttcttcccctccgccgtcgccggcaagccgcgcgtcgaggtccagggcggcgacctcaggtccttcttcacattggtgatgactgaccctgatgtgccagggcctagtgatccatacctgagggagcatcttcactggattgttactgatattcctgggactactgatgcctcttttgggagggaggtggtgagctacgagagcccgcggccaaacatcggcatccacaggttcatcctggtgctgttccggcagaagcgccggcaggcggtgagcccgccgccgtcgagggaccgcttcagcacccgccagttcgccgaggacaacgacctcggcctccccgtcgccgccgtctacttcaacgcgcagcgcgagaccgccgctcgccgccgctaa

SEQ ID NO: 72 - protein, 173 aa, Oryza sativa
MSRVLEPLIVGKVIGEVLDNFNPTVKMTATYGANKQVFNGHEFFPSAVAGKPRVEVQGGDLRSFFTLVMTDPDVPGPSDPYLREHLHWIVTDIPGTTDASFGREVVSYESPRPNIGIHRFILVLFRQKRRQAVSPPPSRDRFSTRQFAEDNDLGLPVAAVYFNAQRETAARRR

SEQ ID NO: 73 - DNA, 537 bp, Oryza sativa
atggccggcagcggcagggacgatcctcttgtggttggcaggattgtgggtgatgtgctggatccattcgtccggatcactaacctcagtgtcagctatggtgcaaggatcgtctccaatggctgcgagctcaagccgtccatggtgacccaacagcccagggtcgtggtcggtggcaatgacatgaggacgttctacacactcgtgatggtagacccggatgctccgagcccaagcaaccctaaccttagggagtatctacactggctggtcaccgatattcctggtaccactggagcaacatttgggcaagaggtgatgtgctacgagagcccaaggccaaccatggggatccaccggctggtgttcgtgctgttccagcagctggggcgtcagacggtgtacgcaccggggtggcgccagaacttcagcaccaggaacttcgccgagctctacaacctcggctcgccggtcgccaccgtctacttcaactgccagcgcgaggccggctccggcggcaggagggtctacccctag

SEQ ID NO: 74 - protein, 178 aa, Oryza sativa
MAGSGRDDPLVVGRIVGDVLDPFVRITNLSVSYGARIVSNGCELKPSMVTQQPRVVVGGNDMRTFYTLVMVDPDAPSPSNPNLREYLHWLVTDIPGTTGATFGQEVMCYESPRPTMGIHRLVFVLFQQLGRQTVYAPGWRQNFSTRNFAELYNLGSPVATVYFNCQREAGSGGRRVYP

SEQ ID NO: 75 - DNA, 552 bp, Oryza sativa
atgtctggtgtgccaactgtggagcccttggttttggctcatgtcatacatgacgtgttagatccatttagaccaactatgccccttagaataacatacaacgataggttacttctggcaggtgctgagctgaaaccatctgcaactgtgcataaaccaagagtagatattggtggcaccgacctgagggtgttctacacattggtactggtggatccagatgctccaagcccaagcaacccatcactaggggagtatttgcactatctccactggatggtgatagatatcccaggaacaactgagtcaactttatcccaagacctcatgctttatgaaagaccggaactgagatatggtatccaccggatggtatttgtgttattccgacaacttggcaggggaaccgtttttgcaccagagatgcgacacaacttccattgtagaagctttgcgcaacaataccatctggacattgtggccgctacatatttcaactgccaaagggaagccggctctggtggaagaaggttcaggtccgagagttcttaa

SEQ ID NO: 76 - protein, 183 aa, Oryza sativa
MSGVPTVEPLVLAHVIHDVLDPFRPTMPLRITYNDRLLLAGAELKPSATVHKPRVDIGGTDLRVFYTLVLVDPDAPSPSNPSLGEYLHYLHWMVIDIPGTTESTLSQDLMLYERPELRYGIHRMVFVLFRQLGRGTVFAPEMRHNFHCRSFAQQYHLDIVAATYFNCQREAGSGGRRFRSESS

SEQ ID NO: 77 - DNA, 531 bp, Oryza sativa
atgagcgggcgggggaggggggacccgctggtgctggggagggtggtgggggacgtggtggacccgttcgtgaggagggtggcgctgcgggtggcgtacggagcgcgggaggtggccaacggctgcgagctccgcccctccgccgtcgccgaccagccccgcgtcgccgtcggcggccccgacatgcgcaccttctacaccctggtgatggtggatccggacgcgccgagcccgagcgatccaaacctcagggagtacctgcactggctggtcaccgacatcccggctaccacaggagtctcttttgggacagaggtggtgtgctacgagagcccgcggccggtgctggggatccacaggctggtgttcctgctgttcgagcagctggggcggcagacggtgtacgcaccggggtggcgccagaacttcagcacccgcgacttcgccgagctctacaacctcggcctccctgtcgccgccgtctacttcaactgccagagggagtctggaaccggaggaagaagaatgtga

SEQ ID NO: 78 - protein, 176 aa, Oryza sativa
MSGRGRGDPLVLGRVVGDVVDPFVRRVALRVAYGAREVANGCELRPSAVADQPRVAVGGPDMRTFYTLVMVDPDAPSPSDPNLREYLHWLVTDIPATTGVSFGTEVVCYESPRPVLGIHRLVFLLFEQLGRQTVYAPGWRQNFSTRDFAELYNLGLPVAAVYFNCQRESGTGGRRM

SEQ ID NO: 79 - DNA, 525 bp, Oryza sativa
atggcccgtttcgtggatccgctggtggtgggacgggtgatcggggaggtggtggatttgttcgttccatccatctccatgaccgccgcctacggcgacagggacatcagcaacggctgcctcgtccgcccatccgccgccgactaccctcccctcgtccgcatctccggccgccgcaacgacctctacaccctgatcatgacggacccggacgcacctagccctagcgacccatccatgagggagtttctccactggatcgtggttaacataccggggggaacagatgcatctaaaggtgaggagatggtggagtacatggggccacggccgacggtggggatacacaggtacgtgctggtgctgtacgagcagaaggcgcgcttcgtggacggcgcgctgatgccgccggcggacaggcccaacttcaacacaagagcattcgcggcgtaccatcagctcggcctccccaccgccgtcgtccacttcaactcccagagggagcccgccaaccgccgccgctaa

SEQ ID NO: 80 - protein, 174 aa, Oryza sativa
MARFVDPLVVGRVIGEVVDLFVPSISMTAAYGDRDISNGCLVRPSAADYPPLVRISGRRNDLYTLIMTDPDAPSPSDPSMREFLHWIVVNIPGGTDASKGEEMVEYMGPRPTVGIHRYVLVLYEQKARFVDGALMPPADRPNFNTRAFAAYHQLGLPTAVVHFNSQREPANRRR

SEQ ID NO: 81 - DNA, 558 bp, Oryza sativa
atggccaacgattcattggctacagggcgtgtgatcggagatgtcctggatcccttcatcagcaccgtcgatctcaccgtcatgtatggtgatgatggcatgccggtcataagcggcgtggagcttcgcgcaccggcggtcgcggagaaaccggtggtcgaagtcgggggagacgatcttcgcgtcgcatatactctggtgatggttgatcctgatgcacctaaccctagcaatccaactctgagggaatacctccactggatggtgactgacatcccggcttcaaccgatgctacatatgggagggaggtggtgtgctacgagagcccgaacccgacgacggggatccacaggatggtgctggtgctgttccggcagctggggagggagacggtgtacgcgccggcggtgcgccacaacttcaccacccgcgccttcgcccgccgctacaacctcggcgcgcccgtcgccgccgtctacttcaactgccagcgccaggccggctccggcggccggaggttcaccggaccttacacctcccgccgccgccaagcctaa

SEQ ID NO: 82 - protein, 185 aa, Oryza sativa
MANDSLATGRVIGDVLDPFISTVDLTVMYGDDGMPVISGVELRAPAVAEKPVVEVGGDDLRVAYTLVMVDPDAPNPSNPTLREYLHWMVTDIPASTDATYGREVVCYESPNPTTGIHRMVLVLFRQLGRETVYAPAVRHNFTTRAFARRYNLGAPVAAVYFNCQRQAGSGGRRFTGPYTSRRRQA

SEQ ID NO: 83 - DNA, 522 bp, Oryza sativa
atgtctaggtctgtggagcctcttgttgttgggcgggtgatcggagaagttattgattcattcaacccatgtacgaagatgatagtaacctacaattcaaacaagcttgtctttaatggccatgagttctacccatcagcagttgtatctaaaccaagagtcgaggtccaagggggtgatatgcgttctttcttcacattggttatgacagacccagatgtgccaggaccaagtgatccatatctaagggaacacctacattggattgtaactgatatacctggaacaacggatgcctcttttggacgggaaatcataagctatgagagcccaaagcccagcattggtatccacaggttcgtttttgtgctcttcaagcagaagcgtaggcaggctgtagttgtgccatcctctagggatcatttcaatacacgccagtttgctgaggagaacgaacttggccttcctgtcgctgctgtctacttcaatgctcagagagagactgctgccaggagacgctaa

SEQ ID NO: 84 - protein, 173 aa, Oryza sativa
MSRSVEPLVVGRVIGEVIDSFNPCTKMIVTYNSNKLVFNGHEFYPSAVVSKPRVEVQGGDMRSFFTLVMTDPDVPGPSDPYLREHLHWIVTDIPGTTDASFGREIISYESPKPSIGIHRFVFVLFKQKRRQAVVVPSSRDHFNTRQFAEENELGLPVAAVYFNAQRETAARRR

SEQ ID NO: 85 - DNA, 522 bp, Oryza sativa
atgtctaggtctgtggagcctcttgttgtagggcgcgtgattggggaagttcttgatacctttaacccatgcatgaagatgatagtgacctataactccaacaagcttgtatttaatggtcatgagctctacccatcagcagttgtgtctaaaccaagagttgaggtccaagggggtgacctgcgatctttcttcacattggttatgacagacccagatgtgccaggaccaagtgatccttatctaagggagcaccttcattggattgttactgatatacctgggacaacggatgcttcttttgggcgcgaggtcataagctatgagagtccaaagccgaacattggcatccataggttcatttttgtgctcttcaagcagaagcgcaggcaaactgtaattgtgccatccttcagggaccatttcaacacccgccggttcgccgaggagaatgatcttggccttcctgtggctgctgtctacttcaatgcccagagagagactgcagccaggaggcgctga

SEQ ID NO: 86 - protein, 173 aa, Oryza sativa
MSRSVEPLVVGRVIGEVLDTFNPCMKMIVTYNSNKLVFNGHELYPSAVVSKPRVEVQGGDLRSFFTLVMTDPDVPGPSDPYLREHLHWIVTDIPGTTDASFGREVISYESPKPNIGIHRFIFVLFKQKRRQTVIVPSFRDHFNTRRFAEENDLGLPVAAVYFNAQRETAARRR

SEQ ID NO: 87 - DNA, 525 bp, Oryza sativa
atgtctagggacccattggttgtcggtcatgtcgtcggcgatatcgtggacccgttcgtcaccaccgcttcgcttagggtcttctacaacagcaaggagatgacaaatgggtctgagctcaagccatctcaggtgttgaaccaaccaaggatttatatcgaaggtcgcgacatgaggacgctctacacgcttgtaatggtggaccctgatgcaccaagccccagcaaccctactaaaagagagtaccttcattggatggtgacagacattccagagaccactgatgccagatttggtaatgagattgtcccctatgagagcccacgcccaactgcaggcatccatcgcttcgtgttcatcctattcaggcagtcagtcaggcagaccacctatgcaccagggtggcgccaaaacttcaatacaagggactttgctgagctctacaacctcggttcgccggtcgccgcgctcttcttcaactgccagagggagaacggctgtggaggaagaaggtgtgttagatga

SEQ ID NO: 88 - protein, 174 aa, Oryza sativa
MSRDPLVVGHVVGDIVDPFVTTASLRVFYNSKEMTNGSELKPSQVLNQPRIYIEGRDMRTLYTLVMVDPDAPSPSNPTKREYLHWMVTDIPETTDARFGNEIVPYESPRPTAGIHRFVFILFRQSVRQTTYAPGWRQNFNTRDFAELYNLGSPVAALFFNCQRENGCGGRRCVR

SEQ ID NO: 89 - DNA, 525 bp, Oryza sativa
atggatcctttgtacctatctcagatcataccggatgtgttggatccatttatttcaaccatttcactcagagtaacctacaacagcaggctacttctggcaggagcagcgcttaaaccatctgcagttgtaagcaagccacaggttgatgttggtggcaatgacatgagggtttcctacacactggtattggtggatccagatgccccaagcccaagtgacccatcgctgagggagtacttgcactggatggtaacagatatccctgaaacaacttccatcagctttggcgaagagttaatattatatgagaagccagagccaagatcaggcatccatcggatggtatttgtgctgttccgccaacttggcaggcggacagtctttgcaccggaaaaacgacataacttcaactgcagaatttttgcacgccaacaccacctcaacatcgtggctgccacatacttcaactgtcaaagggaggcaggatggggtggaagaaagtttgcgcctgaaggcccttaa

SEQ ID NO: 90 - protein, 174 aa, Oryza sativa
MDPLYLSQIIPDVLDPFISTISLRVTYNSRLLLAGAALKPSAVVSKPQVDVGGNDMRVSYTLVLVDPDAPSPSDPSLREYLHWMVTDIPETTSISFGEELILYEKPEPRSGIHRMVFVLFRQLGRRTVFAPEKRHNFNCRIFARQHHLNIVAATYFNCQREAGWGGRKFAPEGP

SEQ ID NO: 91 - DNA, 522 bp, Oryza sativa
atggcaaatgactcattgacaaggagccatatagttggagatgtgttagaccaattttcaaactcagtgcctctaactgtgatgtatgatgggaggcctgtgtttaatggcaaggagttccgttcctcggcagtctcgatgaaacctagagttgagattggtggcgatgattttcgatttgcctataccctagttatggtggatcctgatgctcctaatcccagcaacccaaccttgagggaatacctgcactggatggtgactgatatcccatcatcgacggacgatagctttgggcgggagatcgtaacatacgaaagcccaagccccaccatgggcatccaccgcatcgtgatggtgttgtatcagcagcttgggcgcggcacggtgttcgcgccgcaggtgcgtcagaacttcaacctgcgcagcttcgcgcgccgtttcaacctcggcaagccggtggccgccatgtacttcaactgccagcgcccgacaggcacaggtgggaggaggccaacctga

SEQ ID NO: 92 - protein, 173 aa, Oryza sativa
MANDSLTRSHIVGDVLDQFSNSVPLTVMYDGRPVFNGKEFRSSAVSMKPRVEIGGDDFRFAYTLVMVDPDAPNPSNPTLREYLHWMVTDIPSSTDDSFGREIVTYESPSPTMGIHRIVMVLYQQLGRGTVFAPQVRQNFNLRSFARRFNLGKPVAAMYFNCQRPTGTGGRRPT

SEQ ID NO: 93 - DNA, 531 bp, Oryza sativa
atggcatcgcatgtggacccgctggtggtggggagggtgatcggcgacgtggtggacctgttcgtgccgacgacggccatgtcggtgcggttcgggaccaaggacctcaccaacggctgcgagatcaagccgtccgtcgccgccgcgccgcccgccgtgcagatcgccggcagggtcaacgagctcttcgctctggtcatgactgatccagatgctcctagccccagcgagccgactatgagagagtggcttcactggctggtggttaacataccaggtggaacagatccttctcaaggggatgtggtggtgccgtacatggggccacggccgccggtggggatccaccgctacgtgatggtgctgttccagcagaaggcgcgcgtggcggcgccgccgcccgacgaggacgccgcgcgcgccaggttcagcacgcgcgccttcgccgaccgccacgacctcggcctccccgtcgccgccctctacttcaacgcccagaaggagcccgccaaccgccgccgccgctactag

SEQ ID NO: 94 - protein, 176 aa, Oryza sativa
MASHVDPLVVGRVIGDVVDLFVPTTAMSVRFGTKDLTNGCEIKPSVAAAPPAVQIAGRVNELFALVMTDPDAPSPSEPTMREWLHWLVVNIPGGTDPSQGDVVVPYMGPRPPVGIHRYVMVLFQQKARVAAPPPDEDAARARFSTRAFADRHDLGLPVAALYFNAQKEPANRRRRY

SEQ ID NO: 95 - DNA, 525 bp, Oryza sativa
atgtcaagggatccacttgttgtaggcaatgtggttggggatatcttggacccatttatcaaatcagcatcactcagagtgctttacagcaatagggaactgactaatggatctgagctcaagccttcacaagtagcgaacgagccaaggattgagattgctggtcgtgacatgaggacactttacactttggtgatggtggatcctgactcaccaagtccaagcaatccaaccaaaagagaataccttcattggttggtgacggacattccagaaacaacaaatgcgagctttggaaatgagatagtcagctatgaaagtccaaagccaacagcgggaatacatcgctttgtctttgtgcttttccgtcaatctgtccaacagaccatttatgcacctggatggcgacaaaattttaacacaagggatttctcggcactttacaacctaggaccaccggtggctgccgtgttcttcaactgccaaagagagaatggttgtggtggcagacgatacattagatga

SEQ ID NO: 96 - protein, 174 aa, Oryza sativa
MSRDPLVVGNVVGDILDPFIKSASLRVLYSNRELTNGSELKPSQVANEPRIEIAGRDMRTLYTLVMVDPDSPSPSNPTKREYLHWLVTDIPETTNASFGNEIVSYESPKPTAGIHRFVFVLFRQSVQQTIYAPGWRQNFNTRDFSALYNLGPPVAAVFFNCQRENGCGGRRYIR

SEQ ID NO: 97 - DNA, 231 bp, Oryza sativa
gatgtggtggtgccgtacatggggccacggccgccggtggggatccaccgctacgtgatggtgctgttccagcagaaggcgcgcgtggcggcgccgccgcccgacgaggacgccgcgcgcgccaggttcagcacgcgcgccttcgccgaccgccacgacctcggcctccccgtcgccgccctctacttcaacgcccagaaggagcccgccaaccgccgccgccgctactag

SEQ ID NO: 98 - protein, 76 aa, Oryza sativa
DVVVPYMGPRPPVGIHRYVMVLFQQKARVAAPPPDEDAARARFSTRAFADRHDLGLPVAALYFNAQKEPANRRRRY

SEQ ID NO: 99 - DNA, 516 bp, Sorghum bicolor
atggcgcggttcgtggatccgctggtggtggggcgggtgatcggcgaggtggtggacctgttcgtgccctccatctccatgaccgtcgcctatggccccaaggacatcagcaacggctgcctcctcaagccgtccgccaccgccgcgccgccgctcgtccgcatctccggccgccgcaacgacctctacacgctgatcatgacggaccctgatgcgcctagccccagcgacccgaccatgagggagtacctccactggatagtgaccaacataccaggaggaacggatgcaagcaaaggtgaggaggtggtggagtacatgggcccgcggccgccggtgggcatccaccgctacgtgctggtgctgttcgagcagaagacgcgcgtgcacgcggaggcgccccgcgagcgcgccaacttcaacacgcgcgcgttcgcggcggcgcacgagctcggcctccccaccgccgtcgtctacttcaacgcgcagaaggagcccgccaaccgccgccgctag

SEQ ID NO: 100 - protein, 171 aa, Sorghum bicolor
MARFVDPLVVGRVIGEVVDLFVPSISMTVAYGPKDISNGCLLKPSATAAPPLVRISGRRNDLYTLIMTDPDAPSPSDPTMREYLHWIVTNIPGGTDASKGEEVVEYMGPRPPVGIHRYVLVLFEQKTRVHAEAPRERANFNTRAFAAAHELGLPTAVVYFNAQKEPANRRR

SEQ ID NO: 101 - DNA, 522 bp, Sorghum bicolor
atgtctagatctgtggagtctctcatagttggtcgggtgattggagaagttctcgactcctttagcccatgtgtgaagatggtagtgacctacaactcaaacaagcttgtcttcaatggccatgagatctacccatcagcagttgtatccaaaccaagagtagaggttcaagggggtgacttgcggtctttcttcacattggttatgacagacccagatgttccagggccaagtgatccatatctaagggagcaccttcactggatcgtgactgatatacctgggacaacagatgcctcattcgggcgagaagttataagctatgagagcccaagacctagcattggtatccacaggttcatttttgttctcttcaagcagaagcgcaggcaaactgtagctatgccatcctccagggaccatttcatcacacgacagtttgctgaggaaaatgatcttggactccctgtagctgctgtctacttcaacgctcagagagaaactgctgctaggaggcgctga

SEQ ID NO: 102 - protein, 173 aa, Sorghum bicolor
MSRSVESLIVGRVIGEVLDSFSPCVKMVVTYNSNKLVFNGHEIYPSAVVSKPRVEVQGGDLRSFFTLVMTDPDVPGPSDPYLREHLHWIVTDIPGTTDASFGREVISYESPRPSIGIHRFIFVLFKQKRRQTVAMPSSRDHFITRQFAEENDLGLPVAAVYFNAQRETAARRR

SEQ ID NO: 103 - DNA, 522 bp, Sorghum bicolor
atgtctagggtgttggaacctctagtcgtcggcaaggtgattggggaagtcatcgacaacttcaaccccacggtgaagatgacggttacctacggctccaacaaccaggtgttcaacggccatgagttctttccgtctgcggttctgtccaagccgcgcgtggaggttcagggcgacgacatgaggtccttcttcacgctggtcatgactgacccagatgtgccagggcctagtgatccatacctgagagagcatctccattggatcgtcactgacattcctggaacaactgatgcttcttttggaacggagttggcgatgtacgagagccccaaaccctacatcggcatccacaggttcgtcttcgtgctgttcaagcagaagagccgccagtcggtgcgcccgccctcgtccagggactacttcagcacccgccgctttgccgccgacaacgatctcggcctcccagtcgctgccgtctacttcaacgcgcagcgggagaccgccgcgcgccgccgctga

SEQ ID NO: 104 - protein, 173 aa, Sorghum bicolor
MSRVLEPLVVGKVIGEVIDNFNPTVKMTVTYGSNNQVFNGHEFFPSAVLSKPRVEVQGDDMRSFFTLVMTDPDVPGPSDPYLREHLHWIVTDIPGTTDASFGTELAMYESPKPYIGIHRFVFVLFKQKSRQSVRPPSSRDYFSTRRFAADNDLGLPVAAVYFNAQRETAARRR

SEQ ID NO: 105 - DNA, 534 bp, Sorghum bicolor
atgtcgtcgagggatccgctagtggttggaagcatcgtgggcgacatcgtggactacttctcagcgtcggcgctgctccgagttatgtacggcgggcgcgagatcacctgcgggtcggagctcaggccgtcccaggtcgccggcgagccgacggtgcacatcaccggaggccgccgcgacgggacgccggcgttctacacactgctgatgctggaccctgatgcgcccagcccaagcaacccgaccaaacgggagtatctccattggttggtgactgatataccagaaggagctggtgccaatcatgggaacgaggtggtggcgtacgagagcccccggccatcggcggggatccaccggttcgtgttcatcgtgttccggcaggagatccggcagttgatatacacgccggggtggcgcgccaacttcacatccagggacttcgccgccagctacagcctcggaccgcctgtcgccgccacttacttcaacttccagagggaggtaggctgcggtggctggaggtacaggtga

SEQ ID NO: 106 - protein, 177 aa, Sorghum bicolor
MSSRDPLVVGSIVGDIVDYFSASALLRVMYGGREITCGSELRPSQVAGEPTVHITGGRRDGTPAFYTLLMLDPDAPSPSNPTKREYLHWLVTDIPEGAGANHGNEVVAYESPRPSAGIHRFVFIVFRQEIRQLIYTPGWRANFTSRDFAASYSLGPPVAATYFNFQREVGCGGWRYR

SEQ ID NO: 107 - DNA, 549 bp, Sorghum bicolor
atggccaacgattccttggttacagctcgtgtcataggagatgtcctggaccccttctacagctccattgatctgatggtgctcttcaatggtatgcccattgtcagcggcatggagttgcgtgctccgacggtctctgagaggccaagggttgagatcggaggagatgactatcgtgttgcttataccctggtgatggttgatcctgatgctccaaacccaagcaacccaaccctaagggagtacctgcactggatggtcactgacattccagcgtcaactgatgacacctacgggcgggaggtgatgtgctacgaggccccaaacccgacgacggggatccaccgcatggtgctggtgctgttccggcagctggggcgggagacggtgtacgcgccgtcctggcgccacaacttcagcacgcgcggcttcgcccgccgctacaacctcggcgcgcccgtcgccgccatgtacttcaactgccagcgccagaacggctccggcggacggaggttcaccggggcctacaccggcggcagacatggttag

SEQ ID NO: 108 - protein, 182 aa, Sorghum bicolor
MANDSLVTARVIGDVLDPFYSSIDLMVLFNGMPIVSGMELRAPTVSERPRVEIGGDDYRVAYTLVMVDPDAPNPSNPTLREYLHWMVTDIPASTDDTYGREVMCYEAPNPTTGIHRMVLVLFRQLGRETVYAPSWRHNFSTRGFARRYNLGAPVAAMYFNCQRQNGSGGRRFTGAYTGGRHG

SEQ ID NO: 109 - DNA, 522 bp, Sorghum bicolor
atgtcaagggtgttggagcctctcattgtggggaaagtgattggtgaggtgctggaccatttcaaccccacggtgaagatggtggtcacctacaactccaacaagcaggtcttcaacggacatgagttcttcccttccgcagtcaccgccaagccgcgtgttgaggtccaagggggtgacctcaggtccttcttcacattggtgatgactgaccctgatgttccaggacctagtgatccctacctgagggagcaccttcactggattgttactgatattcctgggactactgatgcttcttttgggagagaggtggtgagctacgagaccccaaagccaaacattggcatccacaggttcatctttgtgctgttccggcagaagcgccggcaggcggtgaacccgccgtcgtccaaggaccgcttcagcacccgccagttcgctgaggacaacgacctcggcctccccgtcgccgccgtctacttcaacgcacagcgcgagaccgccgcccgccggcgctaa

SEQ ID NO: 110 - protein, 173 aa, Sorghum bicolor
MSRVLEPLIVGKVIGEVLDHFNPTVKMVVTYNSNKQVFNGHEFFPSAVTAKPRVEVQGGDLRSFFTLVMTDPDVPGPSDPYLREHLHWIVTDIPGTTDASFGREVVSYETPKPNIGIHRFIFVLFRQKRRQAVNPPSSKDRFSTRQFAEDNDLGLPVAAVYFNAQRETAARRR

SEQ ID NO: 111 - DNA, 531 bp, Sorghum bicolor
atgtcacgaggaagggatcctttggcattgagccaggtaattggtgatgtgttggatcctttcataaagtcagcaacaatgaggattaattatggtgacaaggagatcacaaatggcactggactacgagcatctgctgtgttcaatgcaccacatgttgagattgaaggccacgaccaaacaaagctctacacacttgttatggtggatcctgatgcaccaagtccgagcaaaccagagtacagggaatatctgcattggttggtgacagatacaccagaggcaagagacatacgttttggcaatgaaatagtcccctatgaaagcccaagaccaccagctggaattcatcgaattgtttttgtgctattcaaacagcaagcaagacaaacagtttatgcaccaggatggcggcaaaatttcaacatcagagacttctcagcaatttacaatcttggagcaccagttgctgcattatacttcaactgccaaaaggaaagcggtgttggtggcagaaggttcctgggatca

SEQ ID NO: 112 - protein, 177 aa, Sorghum bicolor
MSRGRDPLALSQVIGDVLDPFIKSATMRINYGDKEITNGTGLRASAVFNAPHVEIEGHDQTKLYTLVMVDPDAPSPSKPEYREYLHWLVTDTPEARDIRFGNEIVPYESPRPPAGIHRIVFVLFKQQARQTVYAPGWRQNFNIRDFSAIYNLGAPVAALYFNCQKESGVGGRRFLGS

SEQ ID NO: 113 - DNA, 264 bp, Sorghum bicolor
ttggtcactgatattccggcgacgactggagtttcttttgggactgaggttgtgtgctacgagagcccacggccggtgctgggaatccaccggatggtgtttctgctcttccaacagctcggccggcagacggtgtacgccccagggtggcggcagaacttcagcacccgcgacttcgccgagctctacaacctcggcttgccagtggccgccgtttacttcaactgccaaagggagtccggaactggtgggagaagaatgtga

SEQ ID NO: 114 - protein, 87 aa, Sorghum bicolor
LVTDIPATTGVSFGTEVVCYESPRPVLGIHRMVFLLFQQLGRQTVYAPGWRQNFSTRDFAELYNLGLPVAAVYFNCQRESGTGGRRM

SEQ ID NO: 115 - DNA, 258 bp, Sorghum bicolor
cgcgaggtgatatgctacgagagccctcggccgccggcggggatccaccgcgtggtgttcgtgctcttccagcagatggcgcgtggctccgtcgaccagccgccggttctccgccacaacttctgcacccgcaacttcgccgtcgaccacggcctgggcgcccccgtcgccgccgccttcttcacctgccagcccgagggtggcaccggcggccgccgccacgacctccgccagccacggagaccgccggcgtcctag

SEQ ID NO: 116 - protein, 85 aa, Sorghum bicolor
REVICYESPRPPAGIHRVVFVLFQQMARGSVDQPPVLRHNFCTRNFAVDHGLGAPVAAAFFTCQPEGGTGGRRHDLRQPRRPPAS

SEQ ID NO: 117 - DNA, 258 bp, Sorghum bicolor
cgtgagatgatgtgctacgagccccctgccccgtccacgggcatccaccggatggtgctggtgctattccagcagcttggccgtgacacggtgttcgcggcgccgtccaggcgccacaacttcaacacccgtgccttcgcccgccgctacaacctcggcgcgcccgtcgccgccatgttcttcaactgccagcgccagaccggctccggtggccccaggttcaccgggccctacaccagccgccgtcgtgcgggctga

SEQ ID NO: 118 - protein, 85 aa, Sorghum bicolor
REMMCYEPPAPSTGIHRMVLVLFQQLGRDTVFAAPSRRHNFNTRAFARRYNLGAPVAAMFFNCQRQTGSGGPRFTGPYTSRRRAG

SEQ ID NO: 119 - DNA, 246 bp, Sorghum bicolor
gagacggtgatgccatacctgggcccttgcccgccggtgggcatccaccgctacgttctggtggtgtaccagcagaaggcccgcttcagggctccgccggtgctagcaccgggggcggaggtggaggcgtcgcgcgcacggttcaggaaccgcgccttcgccgaccgccatgacctaggcctcccagtcgccgccatgtacttcaacgcgcagaaggagccagcaaaccgccaccgccactactga

SEQ ID NO: 120 - protein, 81 aa, Sorghum bicolor
ETVMPYLGPCPPVGIHRYVLVVYQQKARFRAPPVLAPGAEVEASRARFRNRAFADRHDLGLPVAAMYFNAQKEPANRHRHY

SEQ ID NO: 121 - DNA, 228 bp, Sorghum bicolor
caagaggtgatctgctacgagagccctcggccgaccatggggatccaccgcttcgtgctggtgctgttccagcagctggggcgtcagacggtgtacgccccggggtggcgccagaacttcaacaccagggacttcgccgagctctacaacctgggccctcccgtcgccgccgtctacttcaactgccagcgtgaggccggatctgggggaaggaggatgtactcatga

SEQ ID NO: 122 - protein, 75 aa, Sorghum bicolor
QEVICYESPRPTMGIHRFVLVLFQQLGRQTVYAPGWRQNFNTRDFAELYNLGPPVAAVYFNCQREAGSGGRRMYS

SEQ ID NO: 123 - DNA, 228 bp, Sorghum bicolor
aatgaggtagtaagctatgaaagtccaaagccaagtgctggaatacatcgcttcgtctttgtgctcttccgccaatctgtccggcaaactatttatgcgccaggatggaggcaaaatttcaacacaagagacttctcagcattctacaatctaggaccacctgtggcctcagtgttcttcaactgccaaagggagaatgggtgtggtggcagacgatatattagatga

SEQ ID NO: 124 - protein, 75 aa, Sorghum bicolor
NEVVSYESPKPSAGIHRFVFVLFRQSVRQTIYAPGWRQNFNTRDFSAFYNLGPPVASVFFNCQRENGCGGRRYIR

SEQ ID NO: 125 - DNA, 228 bp, Sorghum bicolor
aatgaaatagttccatatgagagcccacgtccaactgccggaatccatcgctttgcattcgtcttgttcaggcagtcagtcaggcagaccacctatgcgccggggtggagatcaaactttaacacaagggacttcgcagccatctacaaccttggctcccctgtcgctgcagtgtacttcaactgccagagagagaacggctgtggtggaagaaggtacataaggtga

SEQ ID NO: 126 - protein, 75 aa, Sorghum bicolor
NEIVPYESPRPTAGIHRFAFVLFRQSVRQTTYAPGWRSNFNTRDFAAIYNLGSPVAAVYFNCQRENGCGGRRYIR

SEQ ID NO: 127 - DNA, 225 bp, Sorghum bicolor
cgagagctcataccatatgagaacccaagccccaccatgggcatccaccgtattgtcttggtgctctaccagcaactggggcggggcacggtgtttgcaccgcaagtgcgtcaaaacttcaacttgcgcaattttgcacgccgtttcaacctcggcaagcctgtggctgcgatgtacttcaactgccagcggcaaacaggcacaggtgggagaaggttcacttga

SEQ ID NO: 128 - protein, 74 aa, Sorghum bicolor
RELIPYENPSPTMGIHRIVLVLYQQLGRGTVFAPQVRQNFNLRNFARRFNLGKPVAAMYFNCQRQTGTGGRRFT

SEQ ID NO: 129 - DNA, 234 bp, Sorghum bicolor
caggagctcatgttttacgaaaggccagaaccgagatctggtatacaccgcatggtatttgtgctgttccggcaacttggtagggggacagtttttgcaccagacatgcgacataacttcaactgcaagaactttgcacgtcaataccacctagacattgtggctgccacatatttcaactgtcaaagggaagcaggatctggagggagaaggttcaggcccgaaagttcgtaa

SEQ ID NO: 130 - protein, 77 aa, Sorghum bicolor
QELMFYERPEPRSGIHRMVFVLFRQLGRGTVFAPDMRHNFNCKNFARQYHLDIVAATYFNCQREAGSGGRRFRPESS

SEQ ID NO: 131 - DNA, 192 bp, Sorghum bicolor
atgtcaagggacccacttgtagtaggcaacgtagttggagatatcttggatccatttatcaaatcagcatcactcagagtcctatacaacaatagggaactgactaatggatctgagctcaagccatcgcaagtagccaatgaaccaaggattgagattgctggacatgacatgaggaccctttacactttg

SEQ ID NO: 132 - protein, 64 aa, Sorghum bicolor
MSRDPLVVGNVVGDILDPFIKSASLRVLYNNRELTNGSELKPSQVANEPRIEIAGHDMRTLYTL

SEQ ID NO: 133 - DNA, 201 bp, Sorghum bicolor
atgttcaatatgtctagggacccattggtcgtcgggcatgtcgtgggggatattgtggatccattcatcacaactgcatcactgagggtgttctacaacaataaggagatgacaaatggttctgaccttaagccatctcaagtgatgaatgagccaagggtccacatcagtgggcgtgacatgaggactctctacacactt

SEQ ID NO: 134 - protein, 67 aa, Sorghum bicolor
MFNMSRDPLVVGHVVGDIVDPFITTASLRVFYNNKEMTNGSDLKPSQVMNEPRVHISGRDMRTLYTL

SEQ ID NO: 135 - DNA, 255 bp, Sorghum bicolor
atgcagcgcggggacccgctggtggtggggcgcatcatcggcgacgtggtcgacccgttcgtgcgccgggtgccgctccgcgtcgcctacgccgcgcgcgagatctccaacggctgcgagctcaggccctccgccatcgccgaccagccgcgcgtcgaggtcggcggacccgacatgcgcaccttctacaccctcgtgatggtagatcctgatgcgccaagccccagcgatcccaacctcagggagtacctgcac

SEQ ID NO: 136 - protein, 85 aa, Sorghum bicolor
MQRGDPLVVGRIIGDVVDPFVRRVPLRVAYAAREISNGCELRPSAIADQPRVEVGGPDMRTFYTLVMVDPDAPSPSDPNLREYLH

SEQ ID NO: 137 - DNA, 255 bp, Sorghum bicolor
atggcggctaacgattccttggttactgctcatgtgataggagatgtcttggaccccttctatacaaccgttgatatgatgatcctattcgatggtactcctattatcagcggcatggagttgcgtgctccggcggtttctgacaggccaagggttgagattggaggagatgattatcgagttgcatatactctggtgatggtcgatcctgatgctcctaacccaagcaacccaaccttgagggagtacttgcac

SEQ ID NO: 138 - protein, 85 aa, Sorghum bicolor
MAANDSLVTAHVIGDVLDPFYTTVDMMILFDGTPIISGMELRAPAVSDRPRVEIGGDDYRVAYTLVMVDPDAPNPSNPTLREYLH

SEQ ID NO: 139 - DNA, 261 bp, Sorghum bicolor
atgtcgacgacgtcaagggacagcctggtgctggggcgggtggtcggcgacgtggtggaccagttctccgcgacggcggcgctccgggtctcctataacggccggcgcgtcatcaacggctccgacctccggccgtcggcggtggcagcaaggcctcgcatcgagatcgggggcaccgatttcaggcagtcctacacgcttgttatggtggatcctgacgctcccaacccgagcaatccgacgttgagggagtatttgcat

SEQ ID NO: 140 - protein, 87 aa, Sorghum bicolor
MSTTSRDSLVLGRVVGDVVDQFSATAALRVSYNGRRVINGSDLRPSAVAARPRIEIGGTDFRQSYTLVMVDPDAPNPSNPTLREYLH

SEQ ID NO: 141 - DNA, 255 bp, Sorghum bicolor
atggctgcccatgtggacccgctggtggtggggagggtgatcggcgatgtggtggacctgttcgtgccgacggtggccatgtcggtgcgcttcggcaccaaggacgtaaccaacggctgcgagatcaagccatccctcaccgccgctgctccggtcgtccagattgccggcagggccaacgacctcttcaccctggttatgactgacccagatgctccgagccccagcgagccaacgatgagggagttgatccac

SEQ ID NO: 142 - protein, 85 aa, Sorghum bicolor
MAAHVDPLVVGRVIGDVVDLFVPTVAMSVRFGTKDVTNGCEIKPSLTAAAPVVQIAGRANDLFTLVMTDPDAPSPSEPTMRELIH

SEQ ID NO: 143 - DNA, 252 bp, Sorghum bicolor
atggctaatgactctctgacgaggggacacataatcggggatgtcttagacccgtttactagctcagtgcctctaactgkcatgtatgatggcagaccggtgtttgatgggatggagtttcgggcgtcggcggtgtcggtgaaacctagagttgagattggaggtgatgattttcgagtggcctataccctagttatggtggatcctgatgcgcctaatcccagcaaccctaccctacgggaatacttgcat

SEQ ID NO: 144 - protein, 84 aa, Sorghum bicolor
  UNSURE (27)...(27): Xaa = any amino acid
MANDSLTRGHIIGDVLDPFTSSVPLTXMYDGRPVFDGMEFRASAVSVKPRVEIGGDDFRVAYTLVMVDPDAPNPSNPTLREYLH

SEQ ID NO: 145 - DNA, 267 bp, Sorghum bicolor
atggccggcagcggcagggaaagggagacgctggtggttggtagggtggtgggcgacgtgctggaccccttcgtccggaccaccaacctcagggtcagctacggcaccaggaccgtatccaacggctgcgagctcaagccgtccatggtggtgaaccagcccagggtcgaggtcgggggacccgacatgaggaccttctacaccctcgtgatggtcgacccggatgctccgagcccaagcgacccaaatcttagggagtatctgcac

SEQ ID NO: 146 - protein, 89 aa, Sorghum bicolor
MAGSGRERETLVVGRVVGDVLDPFVRTTNLRVSYGTRTVSNGCELKPSMVVNQPRVEVGGPDMRTFYTLVMVDPDAPSPSDPNLREYLH

SEQ ID NO: 147 - DNA, 411 bp, Allium cepa
atgttgcgagagagagtagcaagggatcctctagtcttgggacagataattggagatgttgtggatccgtttaccaaatccgtgaatctcaaagtagtttatggagataaggaagtgagtaatggcacaagacttcgtcaatcgatggttataaatcaaccacgtgttaccattgaaggacgtgactcaaggactctttatagccttgttatgataaaccctgatgcaccaagcccaactaatccaactcatagagaatacttacactggttggtgacggacataccagaaacagtcgatgcaagttatggaaatgagatagtacaatatgagagtccatggacgccaactgggattcatcgaattgtatttgtactattccagcagcaaattcaacaaacggtgtatgca

SEQ ID NO: 148 - protein, 137 aa, Allium cepa
MLRERVARDPLVLGQIIGDVVDPFTKSVNLKVVYGDKEVSNGTRLRQSMVINQPRVTIEGRDSRTLYSLVMINPDAPSPTNPTHREYLHWLVTDIPETVDASYGNEIVQYESPWTPTGIHRIVFVLFQQQIQQTVYA

SEQ ID NO: 149 - DNA, 528 bp, Triticum aestivum
atgcatgcccagcgcggggacccgctggtggtggggcgcgtgatcggcgacgtggtggacccgttcgtgcggcgggtggcgctgcgggtcggctacgcgtccagggacgtggccaacggctgcgagctgaggccgtccgccatcgccgacccgccgcgcgtcgaggtcggcggcccggacatgcgcaccttctacacgctggtgatggtggatccggatgctccaagtcccagcgatcccagccttagggagtacttgcactggctggtcaccgacatcccggcgacgacaggagtgtcttttgggaccgaggtggtgtgctacgagggcccgcggccggtgctcgggatccaccggctggtgttcctgctcttccagcagctgggccgccagacggtgtacgccccggggtggcggcagaacttcagcacccgcgacttcgccgagctctacaacctcggcctgcccgtcgccgccgtctacttcaactgccagagggagaccggaaccggcgggagaaggatgtga

SEQ ID NO: 150 - protein, 175 aa, Triticum aestivum
MHAQRGDPLVVGRVIGDVVDPFVRRVALRVGYASRDVANGCELRPSAIADPPRVEVGGPDMRTFYTLVMVDPDAPSPSDPSLREYLHWLVTDIPATTGVSFGTEVVCYEGPRPVLGIHRLVFLLFQQLGRQTVYAPGWRQNFSTRDFAELYNLGLPVAAVYFNCQRETGTGGRRM

SEQ ID NO: 151 - DNA, 543 bp, Triticum aestivum
atggcagcccatgtggatccccttgtggttgggagggtgatcggtgacgtggtggacatgttcgtgcccaccatgccggtgaccgtgcgcttcgggacgaaggacctgacgaacggctgcgagatcaagccgtccatcgccgacgcggcgccctcgatccagatagccggccgggccggcgatctcttcaccctggttatgactgatccggacgcaccgagccccagcgagccaaccatgaaggagtggcttcactggctggtggttaacatacctggtggatcagatccttctcaaggggaggaggtggtgccctacatgggtccgaagccgccgttgggcatccaccgctacgtgctggtgctgttccagcagaaggcgcgtgtgctggcgccggctcccggcggcgacacagcagcgtcggccatgcgcgcgcggttcagcacccgtgccttcgcagagcgccatgacctggggctccccgtcgccgccatgtacttcaacgcgcagaaggagccggccaaccgccgccgccgctactag

SEQ ID NO: 152 - protein, 180 aa, Triticum aestivum
MAAHVDPLVVGRVIGDVVDMFVPTMPVTVRFGTKDLTNGCEIKPSIADAAPSIQIAGRAGDLFTLVMTDPDAPSPSEPTMKEWLHWLVVNIPGGSDPSQGEEVVPYMGPKPPLGIHRYVLVLFQQKARVLAPAPGGDTAASAMRARFSTRAFAERHDLGLPVAAMYFNAQKEPANRRRRY

SEQ ID NO: 153 - DNA, 519 bp, Glycine max
atggcagcctccgtggatcccctagtggttggtcgcgtgatcggcgatgtggtagacatgttcattccttcagtcaacatgtccgtttactttgggtcgaagcacgtcacaaatggctgtgacatcaagccatccattgccatcagccctcctaagctcaccctcaccggcaacatggataacctctacacactggttatgactgatcctgacgcacctagccccagtgaaccaagcatgcgcgagtggatacattggatcttagttgacatacctggaggaacaaacccatttcgcggaaaagagattgtttcatatgtgggaccaagaccacctattggaatacatcgctatatctttgtgttgtttcaacagaaaggacctttaggtcttgtggagcaaccaccaactcgagcaagcttcaacactcgttattttgccaggcaattggacttgggacttccagtggccactgtctacttcaactctcaaaaagaacctgctgttaagaggcgctga

SEQ ID NO: 154 - protein, 172 aa, Glycine max
MAASVDPLVVGRVIGDVVDMFIPSVNMSVYFGSKHVTNGCDIKPSIAISPPKLTLTGNMDNLYTLVMTDPDAPSPSEPSMREWIHWILVDIPGGTNPFRGKEIVSYVGPRPPIGIHRYIFVLFQQKGPLGLVEQPPTRASFNTRYFARQLDLGLPVATVYFNSQKEPAVKRR

SEQ ID NO: 155 - DNA, 522 bp, Glycine max
atggcaagaatgcctttagagcctctaatagtggggagagtcataggagaagttcttgattcttttaccacaagcacaaaaatgattgtgagttacaacaagaatcaagtctacaatggccatgaactcttcccttccactgtcaacaccaagcccaaggttgagattgagggtggtgatatgaggtccttctttacactgatcatgactgaccctgatgttcctggccctagtgacccttatctgagagagcacttgcactggatagtgacagatattccaggcacaacagatgccacatttgggaaagagttggtgagctatgagatcccaaagcctaatattgggatccataggtttgtgtttgtcctgttcaagcaaaagcgtagacagtgtgttactccacccacttcaagggaccacttcaacacacgcaaattcgcagcagagaacgaccttgccctccctgtggctgctgtctacttcaatgcacagagggaaacggctgcaagaagacgctag

SEQ ID NO: 156 - protein, 173 aa, Glycine max
MARMPLEPLIVGRVIGEVLDSFTTSTKMIVSYNKNQVYNGHELFPSTVNTKPKVEIEGGDMRSFFTLIMTDPDVPGPSDPYLREHLHWIVTDIPGTTDATFGKELVSYEIPKPNIGIHRFVFVLFKQKRRQCVTPPTSRDHFNTRKFAAENDLALPVAAVYFNAQRETAARRR

SEQ ID NO: 157 - DNA, 522 bp, Glycine max
atgtctaggctaatggaacaaccacttgttgtgggaagagtgataggagaagtggttgacattttcagcccaagtgtaagaatgaatgttacatattccactaagcaagttgctaatggtcatgagttaatgccttctactattatggccaagccacgcgttgagattggtggtgatgacatgaggactgcttataccttgatcatgacagacccagatgctccaagtcctagtgatccacatctgagggaacatctccactggacggttacagatatccctggcaccacagatgtctcttttggaaaagagattgtaggctatgagagtccaaagccagtaataggaatccacaggtatgtgttcatcttgttcaagcagagaggaagacaaacagtgaggcctccatcttcaagagaccacttcaacacaaggaggttctcagaagagaatggccttggcctaccagttgctgcagtttacttcaatgctcaaagagagactgctgcaagaaggaggtga

SEQ ID NO: 158 - protein, 173 aa, Glycine max
MSRLMEQPLVVGRVIGEVVDIFSPSVRMNVTYSTKQVANGHELMPSTIMAKPRVEIGGDDMRTAYTLIMTDPDAPSPSDPHLREHLHWTVTDIPGTTDVSFGKEIVGYESPKPVIGIHRYVFILFKQRGRQTVRPPSSRDHFNTRRFSEENGLGLPVAAVYFNAQRETAARRR

SEQ ID NO: 159 - DNA, 225 bp, Glycine max
gaagagattgtctcctatgaaagtccacgtccaatagtagggattcatcgaatagtttttgtgttatttcgtcagctgcgtagactaactctgcaacctccaggctggcgccagaatttcaacactagagactttgctgagatttataatcttggattaccagtagcggccatgtacttcaactgtaaacgagaaaatgatcaaagcagtggaagaagaagataa

SEQ ID NO: 160 - protein, 74 aa, Glycine max
EEIVSYESPRPIVGIHRIVFVLFRQLRRLTLQPPGWRQNFNTRDFAEIYNLGLPVAAMYFNCKRENDQSSGRRR

SEQ ID NO: 161 - DNA, 228 bp, Glycine max
cacgaggttgtaacatatgaaagtccgcgaccgatgatggggattcatcgtttagtgtttgtgttatttcgtcaactgggtagggaaacagtgtatgcaccaggatggcgccagaatttcaacactagagaatttgctgaactctacaaccttggattgccagttgctgctgtctatttcaacattcagagggaatctggctctggtggaagaaggttataccattga

SEQ ID NO: 162 - protein, 75 aa, Glycine max
HEVVTYESPRPMMGIHRLVFVLFRQLGRETVYAPGWRQNFNTREFAELYNLGLPVAAVYFNIQRESGSGGRRLYH

SEQ ID NO: 163 - DNA, 225 bp, Glycine max
ggtaacgaggttgtaagctatgaaagcccacgacccacgatggggattcatcggttggtgtttgtgttattccgtcaacagtttagacagagggtgtatgctcctggatggcgacaaaatttcaataccagagaatttgctgaactttacaaccttggattgccggttgctgctgtcttcttcaactgtcagagggaaagtggctctggtggtagaacattttga

SEQ ID NO: 164 - protein, 74 aa, Glycine max
GNEVVSYESPRPTMGIHRLVFVLFRQQFRQRVYAPGWRQNFNTREFAELYNLGLPVAAVFFNCQRESGSGGRTF

SEQ ID NO: 165 - DNA, 147 bp, Glycine max
atggctgcatccggggatcccctattggttggtcgcgtgataggtgatgtggtagatatgttcattccttccttcaacatgttcgtttactttgggtcggagcatgtcacaaatggctatgacattaagccatccatggccataagc

SEQ ID NO: 166 - protein, 49 aa, Glycine max
MAASGDPLLVGRVIGDVVDMFIPSFNMFVYFGSEHVTNGYDIKPSMAIS

SEQ ID NO: 167 - DNA, 486 bp, Helianthus argophyllus
catatcagcatgtcccttgtcgtagggcgggtgataggtgatgtcgtcgaccaattcacaccaagcgtgtcgatggatgtagtctataatccccagtgcccggtcttaaacggccatgagatcaagcctaatctcattgccactaaacctcgtgttaatatcggcggtgttgacatgagatcatcttatactcttatcatgactgaccccgatgctccaagtccaagtgacccatacttgagagaacatcttcattggattgtcacagacattcctggtacaactgaagcaacttttggaagggagattgggagctatgaaaaaccaaagccagtgataggaatccatcgctatgtgttcttattgctcaagcaaagagctaggcagtcggggaggcgaccagttgtgcgagatcgattcaacactcgtgccttctctcaagaaagagacttggggttacctgttgctgctagctacttccttggg

SEQ ID NO: 168 - protein, 162 aa, Helianthus argophyllus
HISMSLVVGRVIGDVVDQFTPSVSMDVVYNPQCPVLNGHEIKPNLIATKPRVNIGGVDMRSSYTLIMTDPDAPSPSDPYLREHLHWIVTDIPGTTEATFGREIGSYEKPKPVIGIHRYVFLLLKQRARQSGRRPVVRDRFNTRAFSQERDLGLPVAASYFLG

SEQ ID NO: 169 - DNA, 522 bp, Helianthus species
cccaagtgtgttagcatgtcgcttgcagtagggagggtgattggagatgtcgttgacccattcacaccgagtgtgacgatggaagtagcgtataactcccattacacggtctctagtgggcacgagctgatgcctaatatcattacttctaaacctcaagttcatattggcggtgttgacatgcgatctgcttatactattatcttgactgacccggatgcacccagtccgagtgatccttacttgagagaacatctccattggatcgtcacagacattcctggcacaactgatgcaacttttggaagggagattgtgagctatgaaaaaccgaatccacttataggcatccaccgatacgttttcttactattcaaacagagagcaaggaaatcagttaggccacccgcttccagagatcagttcaatacacggaacttctctcaagaaaacgacttagggttaccggttgctgctgtctacttcaatgctcaaagagcaaatgccgcacgtagaagataa

SEQ ID NO: 170 - protein, 173 aa, Helianthus species
PKCVSMSLAVGRVIGDVVDPFTPSVTMEVAYNSHYTVSSGHELMPNIITSKPQVHIGGVDMRSAYTIILTDPDAPSPSDPYLREHLHWIVTDIPGTTDATFGREIVSYEKPNPLIGIHRYVFLLFKQRARKSVRPPASRDQFNTRNFSQENDLGLPVAAVYFNAQRANAARRR

SEQ ID NO: 171 - DNA, 339 bp, Helianthus species
atgtcgaggagggagagggacccgttggtcgttggacgtgtgataggagatgttcttgatagtttcacaaagtcgattaaccttacgatttcttacaacgacagggaagttagcaacgggtgcacactaaaaccctctcaggttgttaaccagcctcgggttgatattggaggtgacgacctacgagcttttcacactttagtcatggtggatcctgatctcccaagtccaagtgaccctaaccttagggaatacttgcattggttggtgactgatattccagcgaccactgggagcacgttttggtcaagaaagttggtgtgctatgagagtccaagg

SEQ ID NO: 172 - protein, 113 aa, Helianthus species
MSRRERDPLVVGRVIGDVLDSFTKSINLTISYNDREVSNGCTLKPSQVVNQPRVDIGGDDLRAFHTLVMVDPDLPSPSDPNLREYLHWLVTDIPATTGSTFWSRKLVCYESPR

SEQ ID NO: 173 - DNA, 534 bp, Arabidopsis thaliana
atggagaatatgggaactagagtgatagagccattgataatggggagagtggtaggagatgttcttgatttcttcactccaacaactaagatgaatgttagttataacaagaagcaagtctccaatggccatgagctctttccttcttctgtttcctccaagcctagggttgagatccatggtggtgatctcagatccttcttcactttggtgatgatagacccagatgttccaggtcctagtgacccctttctaaaagaacacctgcactggatcgttacaaacattcccggcacaacagatgctacgtttggcaaagaggtggtgagctatgaattgccaaggccaagcatagggatacataggtttgtgtttgttctgttcaggcagaagcaaagacgtgttatctttcctaatatcccttcgagagatcacttcaacactcgtaaatttgcggtcgagtatgatcttggtctccctgtcgcggccgtcttctttaacgcacaaagagaaaccgctgcacgcaaacgctag

SEQ ID NO: 174 - protein, 177 aa, Arabidopsis thaliana
MENMGTRVIEPLIMGRVVGDVLDFFTPTTKMNVSYNKKQVSNGHELFPSSVSSKPRVEIHGGDLRSFFTLVMIDPDVPGPSDPFLKEHLHWIVTNIPGTTDATFGKEVVSYELPRPSIGIHRFVFVLFRQKQRRVIFPNIPSRDHFNTRKFAVEYDLGLPVAAVFFNAQRETAARKR

SEQ ID NO: 175 - DNA, 528 bp, Arabidopsis thaliana
atggccaggatttcctcagacccgcttatggttgggagagtgatcggagacgttgtggacaattgtttgcaggcagtgaaaatgacggtgacctataattctgacaagcaagtctacaatggccatgaacttttcccttctgtagttacatacaaacctaaggttgaagttcatgggggtgacatgagatcattcttcactttggttatgactgatcctgatgttcctggacctagtgatccttatctgagagagcacttgcactggattgttaccgatatcccggggacgactgatgtatcatttggtaaagagataatcgggtacgagatgcctcggccaaacatagggatccaccgctttgtgtatttgttgttcaagcagacccgtagaggaagtgtggtgtctgtgccatcttacagagaccaattcaacactcgagagtttgctcatgagaacgatcttggcctccccgtcgcggctgttttcttcaactgccagcgtgagaccgccgctagacgccgttga

SEQ ID NO: 176 - protein, 175 aa, Arabidopsis thaliana
MARISSDPLMVGRVIGDVVDNCLQAVKMTVTYNSDKQVYNGHELFPSVVTYKPKVEVHGGDMRSFFTLVMTDPDVPGPSDPYLREHLHWIVTDIPGTTDVSFGKEIIGYEMPRPNIGIHRFVYLLFKQTRRGSVVSVPSYRDQFNTREFAHENDLGLPVAAVFFNCQRETAARRR

SEQ ID NO: 177 - DNA, 534 bp, Arabidopsis thaliana
atgtcaagagaaatagagccactaatagtgggaagagtgataggagatgtactcgaaatgtttaatccaagtgtgacaatgagagtcactttcaattccaacacaatcgtatccaatggtcacgagctcgcgccttctcttctcctctctaagcctcgcgttgagatcggtggccaagatcttcgttccttcttcaccttaatcatgatggaccccgatgccccgagtcctagtaatccttatatgcgtgaatatctgcattggatggtgacagatattcccgggacaaccgatgcttcttttgggagagagatagtgagatatgagacgcctaaaccggtggcgggaatacacagatacgtctttgcgctattcaaacagagagggaggcaagctgtgaaggcagcgccggaaactagagagtgtttcaacacaaacgctttctcttcttactttggtctttctcaacctgttgctgctgtttacttcaacgcccaacgtgaaactgctcctcgacgacgtccttcttattaa

SEQ ID NO: 178 - protein, 177 aa, Arabidopsis thaliana
MSREIEPLIVGRVIGDVLEMFNPSVTMRVTFNSNTIVSNGHELAPSLLLSKPRVEIGGQDLRSFFTLIMMDPDAPSPSNPYMREYLHWMVTDIPGTTDASFGREIVRYETPKPVAGIHRYVFALFKQRGRQAVKAAPETRECFNTNAFSSYFGLSQPVAAVYFNAQRETAPRRRPSY

SEQ ID NO: 179 - DNA, 528 bp, Arabidopsis thaliana
atgtctataaatataagagaccctcttatagtaagcagagttgttggagacgttcttgatccgtttaatagatcaatcactctaaaggttacttatggccaaagagaggtgactaatggcttggatctaaggccttctcaggttcaaaacaagccaagagttgagattggtggagaagacctcaggaacttctatactttggttatggtggatccagatgttccaagtcctagcaaccctcacctccgagaatatctccattggttggtgactgatatccctgctacaactggaacaacctttggcaatgagattgtgtgttacgaaaatccaagtcccactgcaggaattcatcgtgtcgtgtttatattgtttcgacagcttggcaggcaaacagtgtatgcaccagggtggcgccagaacttcaacactcgcgagtttgctgagatctacaatctcggccttcccgtggccgcagttttctacaattgtcagagggagagtggctgcggaggaagaagactttag

SEQ ID NO: 180 - protein, 175 aa, Arabidopsis thaliana
MSINIRDPLIVSRVVGDVLDPFNRSITLKVTYGQREVTNGLDLRPSQVQNKPRVEIGGEDLRNFYTLVMVDPDVPSPSNPHLREYLHWLVTDIPATTGTTFGNEIVCYENPSPTAGIHRVVFILFRQLGRQTVYAPGWRQNFNTREFAEIYNLGLPVAAVFYNCQRESGCGGRRL

SEQ ID NO: 181 - DNA, 528 bp, Arabidopsis thaliana
atgtctttaagtcgtagagatcctcttgtggtcggcagtgttgttggagatgttcttgatcctttcacgaggttggtctctcttaaggtcacttatggccatagagaggttactaatggcttggatctaaggccttctcaagttctgaacaaaccaatagtggagattggaggagacgacttcagaaatttctacaccttggttatggtggatccagatgtgccgagtccaagcaaccctcaccaacgagaatatctccactggttggtgactgatatacctgccaccactggaaatgcctttggcaatgaggtggtgtgctacgagagtccacgtcccccctcgggaattcatcgtattgtgttggtattgttccggcaactcggaagacaaacggtttatgcaccggggtggcgccaacagttcaacactcgtgagtttgctgagatctacaatcttggtcttcctgtggctgcctcttacttcaactgccagagggagaatggctgtgggggaagaagaacgtag

SEQ ID NO: 182 - protein, 175 aa, Arabidopsis thaliana
MSLSRRDPLVVGSVVGDVLDPFTRLVSLKVTYGHREVTNGLDLRPSQVLNKPIVEIGGDDFRNFYTLVMVDPDVPSPSNPHQREYLHWLVTDIPATTGNAFGNEVVCYESPRPPSGIHRIVLVLFRQLGRQTVYAPGWRQQFNTREFAEIYNLGLPVAASYFNCQRENGCGGRRT

SEQ ID NO: 183 - DNA, 522 bp, Arabidopsis thaliana
atggcggcttctgttgatcctttggtggtcggaagagtgatcggagatgtgttggacatgttcatcccaaccgccaatatgtctgtctactttggccccaaacacatcactaacggctgcgagatcaaaccctccaccgcagtcaatcctccaaaagtcaacatctcgggccattccgatgagctttacactctcgtgatgactgacccggacgcacctagcccaagcgagccgaacatgagagaatgggtccactggattgtcgtggatattcccggaggcacaaatccctcaagaggaaaagagatacttccatacatggaaccaaggccaccagtggggattcaccgttacatattggtacttttccggcaaaactcaccggtgggtctgatggtgcagcagcctccatcacgagccaatttcagcacacgaatgttcgctggacatttcgatcttggtctacctgtggccactgtctatttcaacgcccaaaaggaacctgcttcacgcagacgctag

SEQ ID NO: 184 - protein, 173 aa, Arabidopsis thaliana
MAASVDPLVVGRVIGDVLDMFIPTANMSVYFGPKHITNGCEIKPSTAVNPPKVNISGHSDELYTLVMTDPDAPSPSEPNMREWVHWIVVDIPGGTNPSRGKEILPYMEPRPPVGIHRYILVLFRQNSPVGLMVQQPPSRANFSTRMFAGHFDLGLPVATVYFNAQKEPASRRR

(19) United States
(12) Patent Application Publication
(10) Pub. No.: US 2008/0178353 A1
(43) Pub. Date: Jul. 24, 2008

(54) Methods for Improving Crop Plant Architecture and Yield

(75) Inventors: Olga N. Danilevskaya, Johnston, IA (US); Evgueni Ananiev, Johnston, IA (US); Carl R. Simmons, Des Moines, IA (US); Pedro Hermon, Johnston, IA (US); Rajeev Gupta, Johnston, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: PIONEER HI-BRED INTERNATIONAL, INC., Johnston, IA (US)

(21) Appl. No.: 12/056,469

(22) Filed: Mar. 27, 2008

Related U.S. Application Data
(63) Continuation of application No. 11/433,973, filed on May 15, 2006.
(60) Provisional application No. 60/684,617, filed on May 25, 2005.

(51) Int. Cl. C12N 15/29; A01H 5/00; C12N 5/10; C12N 1/21

(52) U.S. Cl. 800/290; 800/278; 800/320.1; 800/298; 536/23.6; 435/419; 435/252.3; 800/312; 800/322; 800/320; 800/320.3; 800/320.2; 800/314

(57) ABSTRACT

The present invention provides methods for altering plant characteristics by introducing into plants, isolated nucleic acid molecules that can be used to produce transgenic plants characterized by altered plant architecture, plant maturity, carbon and nitrogen partitioning and or improved harvestable yield. Also provided are isolated nucleic acids that encode PDR polypeptides, vectors capable of expressing such nucleic acid molecules, host cells containing such vectors, and polypeptides encoded by such nucleic acids.

### CROSS REFERENCE

This utility application is a continuation of and claims the benefit of U.S. Non-provisional application Ser. No. 11/433,973, filed May 15, 2006, and claims the benefit U.S. Provisional Application No. 60/684,617, filed May 25, 2005, which are each incorporated herein by reference.

### FIELD OF THE INVENTION

The present invention is drawn to plant genetics and molecular biology. More particularly, the methods involve improving architecture and yield in plants by modulating the expression of nucleic acids within plants. This invention describes a method for improving crop plant architecture and yield in transgenic plants by manipulation of PDR genes which are homologues of the CETS gene family.

### BACKGROUND

The CETS gene family (Pnueli, et al., 2001) was named for the three plant genes: Antirrhinum CENTRORADIALIS (CEN) (Bradley, et al., 1996), Arabidopsis TERMINAL FLOWER 1 (TF1) (Bradley, et al., 1997), and tomato SELF-PRUING (SP) (Pnueli, et al., 1998). The CETS homologues are designated PDR (for Plant Developmental Regulators), based upon the expanded knowledge gained about these genes. The three-letter designation is in accordance with plant gene naming standards (Plant Journal, (1997) 12:247-253). The CETS (PDR) genes encoded closely related proteins with similarity to mammalian phosphatidylethanolamine-binding proteins (PEBPs) (Kardailsky, et al., 1999; Kobayashi, et al., 1999). Mammalian PEBS proteins have been found to act as inhibitors of MAP kinase signaling. The first studied is RKIP (Raf kinase inhibitor protein) which plays a pivotal role in several protein kinase signaling cascades (Yeung, et al., 1999; Lorenz, et al., 2003). The 3-dimensional structure of the CEN protein suggests that plant CETS/PDR protein interfering with kinases like their mammalian counterparts (Banfield, et al., 1998; Banfield and Brady, 2000). Biochemical properties of the CETS/PDR protein family indicate their potential roles as modulators of hormone signaling cascades controlling cell growth and differentiation. Being kinase inhibitors/effectors, the CETS/PDR might be involved in regulation of diverse genetic pathways working as modulators of signaling from hormones to target genes in the various cell types or tissues.

Mutational analysis of the CETS/PDR genes in several dicot species has revealed their function in determining the fate of meristem. One group of CETS/PDR genes, such as the LF (Late Flowering) from pea (Foucher, et al., 2003) and the TFL1 from Arabidopsis (Bradley et al., 1997), act as repressors of flowering by maintaining the apical meristem in the vegetative state. The second group of CETS/PDR genes, including DET (DETERMINATE) from pea (Foucher, et al., 2003), CEN from snapdragon (Cremer, et al., 2001), SP (SELF-PRUNING) from tomato (Pnueli, et al., 1998), and TFL1 from Arabidopsis, maintain indeterminancy of the inflorescence meristem by delaying its transition to the flowers. The Arabidopsis TFL1 gene plays a dual role by controlling the length of both vegetative and floral phases (Ratcliffe, et al., 1998). The Arabidopsis FT (FLOWERING LOCUS T) gene belongs to the CETS gene family as well, but it has a TFL1-antagonist role by promoting flowering, hence, accelerating the transition from vegetative to reproductive phase (Kardailsky, et al., 1999; Kobayashi, et al., 1999). The cited literature describes the role of the PDR genes in maintaining the indeterminancy of the shoot apical and inflorescence meristems explaining their roles in controlling flowering time, and the “determinate habit” of shoot growth.

Dicotocyledoneous plants such as arabidopsis, tomato and poplar appear to possess a small PDR gene family of six to eight genes depending upon the species (Mimida, et al., 2001; Carmel-Goren, et al., 2003; Kotada, 2005). Consistent with this observation, further analysis revealed 7 PDR genes in soybean. Additional studies have revealed a larger family of the PDR genes in monocot genomes. There are more than 22 PDR genes in the rice and maize genomes. Gene expression analysis performed on the genome-wide scale by MPSS RNA profiling suggests functional diversity of the maize PDR genes. Based on a tissue specific pattern of expression, one finds novel functions for the PDR genes, namely involvement in kernel, leaf and vascular bundle development, and drought stress response. Because of their apparently wider functional roles, the maize PDR genes may be used in genetically modified plants for more diverse outcomes, ranging from improving grain yield, stalk strength, plant biomass, canopy shape and drought tolerance. Because of the high similarity of amino acid sequences of the PDR proteins, judiciously altered ectopic expression of the gene family members may allow for the genes to cross their normal functional roles and affect a number of agronomic traits.

Experiments have demonstrated that ZmPDR01 and ZmPDR02 when linked to a constitutive promoter such as UBI lead to enhancement of multiple agronomic traits in transgenic plants. Maize ZmPDR01 transgenic plants showed on average 22% more spikelets per ear, 78% more spikelets per tassel, 20% larger leaf area, 17% leaf angle increase, and 30% stronger stalks. A large number of valuable agronomic traits have been changed by the action of one protein. The spikelet count per ear is a primary grain yield component. The ZmPDR01 gene, therefore, acts as a genetic factor regulating the ear length which increases the yield potential. Transgenic plants also have a favorable canopy shape, copious pollen and increased stalk strength. Together, these transgene-induced traits will support development of higher yielding varieties and hybrids (FIG. 1).

Grain yield in corn is defined as weight of grain harvested per unit area (Duvick, 1992). Yield is one of the most complex agronomic traits and is determined by the interaction of specific genetics within the crops with environmental factors.

There are two general approaches to increasing yield potential: 1) increasing overall plant productivity to increase harvestable yield, and 2) overcoming the negative consequences of any abiotic stresses. Several yield components are critical for harvestable yield in maize: kernel number per ear, photosynthetic capacity, canopy shape, and standability. In the past yield increases have been achieved by breeding efforts via a number of incremental, consecutive steps (Duvick, 1992). The transgenic manipulation of the PDR genes provide a method for improving several yield components, such as kernel number, canopy shape, stalk strength, and vegetative biomass in a single larger step. Also, there is a potential for increased drought tolerance by manipulation of the appropriate class of the maize PDR genes responsive to water availability. Therefore, PDR genes are powerful morphology controlling genes that allow genetic modification of several critical yield components causing increases in both plant productivity and stress tolerance.

### SUMMARY OF THE INVENTION

Compositions and methods for improving crop plant architecture and yield by manipulation of PDR gene family in transgenic plants are provided (FIG. 1).

The present invention provides polynucleotides, related polypeptides and conservatively modified variants of the PDR sequences. The polynucleotides and polypeptides of the invention include PDR genes, proteins and functional fragments or variants thereof.

The methods of the invention comprise introducing into a plant a polynucleotide and expressing the corresponding polypeptide within the plant. The sequences of the invention can be used to alter plant cell growth, leading to changes in plant structural architecture, thereby improving plant yield. The methods of the invention find use in improving plant structural characteristics, leading to increased yield.

Additionally provided are transformed plants, plant tissues, plant cells, seeds, and leaves. Such transformed plants, tissues, cells, seeds, and leaves comprise stably incorporated in their genomes at least one polynucleotide molecule of the invention.

One embodiment of the invention is a method for plant characteristics, the method comprising:
a. introducing into a plant cell a recombinant expression cassette comprising a polynucleotide whose expression, alone or in combination with additional polynucleotides, functions as a plant developmental regulator polypeptide within the plant;
b. culturing the plant cell under plant forming conditions to produce a plant; and,
c. inducing expression of the polynucleotide for a time sufficient to alter the architecture of the plant.

A second embodiment would be a method for increasing plant harvestable yield, the method comprising:
a. introducing into a plant cell a recombinant expression cassette comprising a polynucleotide whose expression, alone or in combination with additional polynucleotides, functions as a plant developmental regulator polypeptide within the plant;
b. culturing the plant cell under plant forming conditions to produce a plant; and,
c. inducing expression of the polynucleotide for a time sufficient to increase the harvestable yield of the plant.

A third embodiment would include an isolated polynucleotide selected from the group consisting of:
a. a polynucleotide having at least 80% sequence identity, as determined by the GAP algorithm under default parameters, to the full length sequence of a polynucleotide selected from the group consisting of SEQ ID NOS: 51, 89, 91, 97, 119, 127, 139, 147, 165 and 171; wherein the polynucleotide encodes a polypeptide that has PDR functions; and
b. a polynucleotide encoding a polypeptide selected from the group consisting of SEQ ID NO: 50, 90, 92, 98, 120, 128, 140, 148, 166 and 170; and
c. a polynucleotide selected from the group consisting of SEQ ID NOS: 51, 89, 91, 97, 119, 127, 139, 147, 165 and 171; and
d. a polynucleotide which is complementary to the polynucleotide of (a), (b) or (c).

### BRIEF DESCRIPTION OF THE FIGURES

FIG. 1—Diagram depicting improving crop plant architecture and yield by manipulation the PDR genes in transgenic plants.

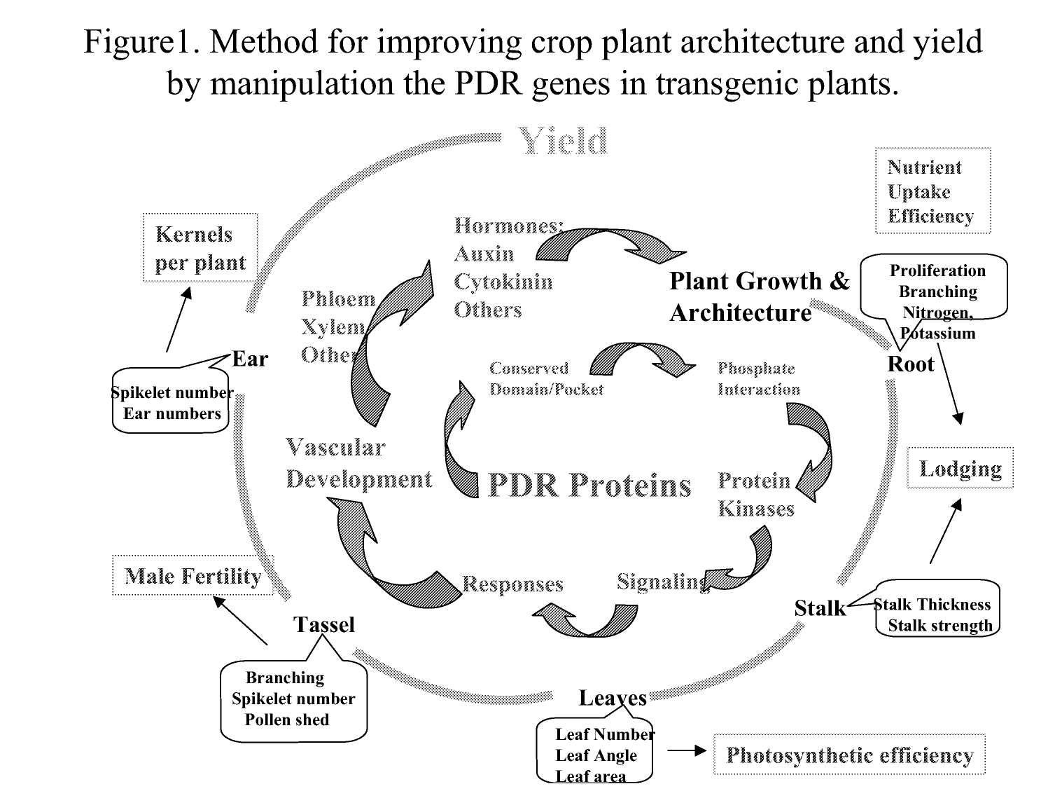

FIG. 2—Photographic demonstration of the altered traits of genetically modified ZmPDR01 (PHP21051) transgenic plants. Transgenic plants (T) showed a distinct appearance difference from non-transgenic (NT) siblings. The transgenic plants showed a distinct canopy shape with upright wide leaves, tassels with high spikelet density and copious pollen shed, and elongated ears.

FIG. 3—Diagrammatic representation and photographic evidence of increased spikelet density on tassel branches of ZmPDR01 (PHP21051). Side by side comparison of the central spikes in control Gaspe (GASPE) and transgenic Gaspe (GASPE UBI::ZmPDR01) revealed that the distance between adjacent whorls of rachillas in transgenic Gaspe is almost half that of control plants, leading to a doubled number of spikelets. Gaspe UBI::ZmPDR1 tassel inflorescence meristems produced approximately 2 times more SPMs (spikelet pair meristems) per unit length than control GASPE plants.

FIG. 4—Photographic evidence of increased vascular bundle size in a stalk of ZmPDR01 (PHP21051) transgenic plants. Side by side comparison of the stalk cross-sections at the 1^{st} internode in control Gaspe (GASPE) and transgenic Gaspe (GASPE UBI::ZmPDR01) revealed that numbers of vascular bundles, as well as their size are increased in transgenic plants.

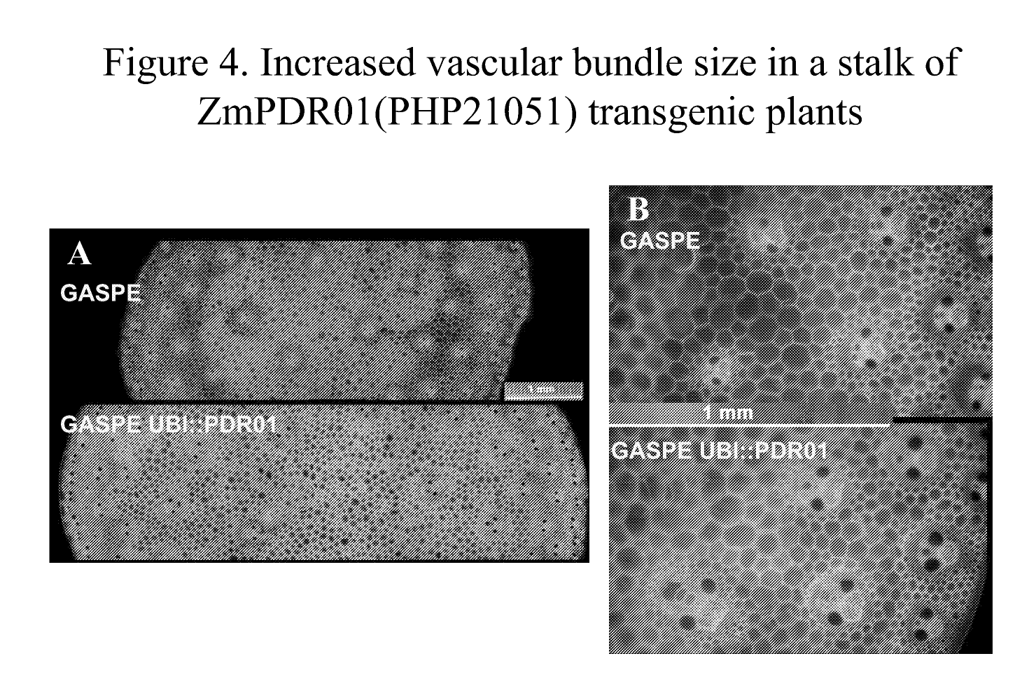

FIG. 5—Structural superimposition of (a) and (d) CEN/ZmPDR01 and (b) and (e) ZmPDR01/ZmPDR14 and (c) ZmPDR01's ligand binding cavity. The three-dimensional structure of ZmPDR01 and ZmPDR14 proteins suggests their function as kinase effectors/regulators.

FIG. 6—Phylogenetic tree representing the Arabidopsis (At) PDR proteins. Mouse PEPB protein was used to outgroup. Three clades were delineated: the FT clade, the PDR1 clade, and the MFT clade.

FIG. 7—Phylogenetic tree for the Soybean (Gm) and Arabidopsis (At) PDR proteins. Seven soybean PDR genes were identified. The GmPDR genes are grouped into one of 3 Arabidopsis clades (FT, PDR and MFT).

FIG. 8—Phylogenetic tree for the Rice (Os) and Arabidopsis (At) PDR proteins. Twenty-two full-length proteins from Rice were identified. The phylogenetic tree includes 4 clades, three clades as described for dicots (FT, PDR1, MFT) and a fourth monocot lade (MC).

FIG. 9—Phylogenetic tree for the Maize (Zm) and Arabidopsis (At) PDR proteins. Eighteen full length proteins from Maize were identified. The phylogenetic tree includes 4 clades, FT, PDR1, MFT and MC.

FIG. 10—MPSS (Massively Parallel Signature Sequencing) profiling data for RNA tissue specific expression patterns/predicted function for maize PDR genes. FIG. 10A is the TFL1 clade, FIG. 10B is the MFT clade, FIG. 10C is the FT clade, and FIG. 10D is the MC clade.

FIG. 11—In situ hybridization of ZmPDR01 to the shoot apical meristem. The hybridization revealed a strong signal of the ZmPDR01 antisense RNA in vascular bundles. Hybridization signal was found in the primordial provascular cells which surround mature vascular bundles with differentiated phloem and zylem. FIG. 11A (transverse section) shows the ZmPDR01 signal concentrated around vascular bundles in the form of isolated islands. FIG. 11B (longitudinal section) shows the ZmPDR01 hybridization signals concentrated in the form of elongated islands around vascular bundles.

FIG. 12—In situ hybridization of ZmPDR01RNA to vascular bundles. Hybridization signal can be detected in vascular bundles with well-developed xylem vessels which are visualized by UV illumination. No obvious signal is seen in the phloem or companion cells. ZmPDR01 could be involved in the control of provascular and protoxylem cell identity.

FIG. 13—Comparison of in situ hybridization of ZmPDR02, ZmPDR04 and ZmPDR05 to ear tips. The hybridization patterns of these three genes from the TFLlclade were analyzed under dark field to visualize hybridization signals and UV illumination to visualize vascular bundles 13A, B, A′, B′. ZmPDR02 and ZmPDR04 are expressed in groups of cells underlying the first 8-9 consecutive spikelets from the top in each row. At the lower part of the ear hybridization signals are overlapped with lignified xylems. 13C,C′ —ZmPDR05 is expressed in groups of cells underlying the earliest spikelet pair meristems as well as in the first 8-10 consecutive spikelets from the top of each row. At the lower part of the ear expression of the ZmPDR05 can be detected mostly in groups of cells tightly associated with vascular bundles (phloem).

FIG. 14—In situ hybridization of ZmPDR02 to the inner side of the vascular bundles and spikelet vasculature. Cells showing expression of ZmPDR02 include protoxylem (px), spikelet vascular bundles (svb), and gynocium (gy). FIG. 14A is dark field, FIG. 14B is UV illumination.

FIG. 15—In situ hybridization of ZmPDR05 to the outer side of the vascular bundles. PDR ZmPDR05 expressing cells are seen in vascular bundles in both 15A (dark field) and 15B (UV illumination) views.

### DETAILED DESCRIPTION OF THE INVENTION

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the invention.

The present inventions now will be described more fully hereinafter with reference to the accompanying drawings, in which some, but not all embodiments of the invention are shown. Indeed, these inventions may be embodied in many different forms and should not be construed as limited to the embodiments set forth herein. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The practice of the present invention will employ, unless otherwise indicated, conventional techniques of botany, microbiology, tissue culture, molecular biology, chemistry, biochemistry and recombinant DNA technology, which are within the skill of the art. Such techniques are explained fully in the literature. See, e.g., Langenheim and Thimann, BOTANY: PLANT BIOLOGY AND ITS RELATION TO HUMAN AFFAIRS, John Wiley (1982); CELL CULTURE AND SOMATIC CELL GENETICS OF PLANTS, vol. 1, Vasil, ed. (1984); Stanier, et al., THE MICROBIAL WORLD, 5^{th} ed., Prentice-Hall (1986); Dhringra and Sinclair, BASIC PLANT PATHOLOGY METHODS, CRC Press (1985); Maniatis, et al., MOLECULAR CLONING: A LABORATORY MANUAL (1982); DNA CLONING, vols. I and II, Glover, ed. (1985); OLIGONUCLEOTIDE SYNTHESIS, Gait, ed. (1984); NUCLEIC ACID HYBRIDIZATION, Hames and Higgins, eds. (1984); and the series METHODS IN ENZYMOLOGY, Colowick and Kaplan, eds, Academic Press, Inc., San Diego, Calif.

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

In describing the present invention, the following terms will be employed, and are intended to be defined as indicated below.

By “microbe” is meant any microorganism (including both eukaryotic and prokaryotic microorganisms), such as fungi, yeast, bacteria, actinomycetes, algae and protozoa, as well as other unicellular structures.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., DIAGNOSTIC MOLECULAR MICROBIOLOGY: PRINCIPLES AND APPLICATIONS, Persing, et al., eds., American Society for Microbiology, Washington, D.C. (1993). The product of amplification is termed an amplicon.

The term “conservatively modified variants” applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine; one exception is Micrococcus rubens, for which GTG is the methionine codon (Ishizuka, et al., (1993) J. Gen. Microbiol. 139:425-32) can be modified to yield a functionally identical molecule. Accordingly, each silent variation of a nucleic acid, which encodes a polypeptide of the present invention, is implicit in each described polypeptide sequence and incorporated herein by reference.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7 or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80% or 90%, preferably 60-90% of the native protein for it's native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton, PROTEINS, W.H. Freeman and Co. (1984).

As used herein, “consisting essentially of” means the inclusion of additional sequences to an object polynucleotide where the additional sequences may not selectively hybridize, under stringent hybridization conditions, to the same cDNA as the polynucleotide and where the hybridization conditions include a wash step in 0.1×SSC and 0.1% sodium dodecyl sulfate at 65° C.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolumn (Yamao, et al., (1985) Proc. Natl. Acad. Sci. USA 82:2306-9), or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 5 of Murray, et al., supra.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived or, if from the same species, one or both are substantially modified from their original form. A heterologous protein may originate from a foreign species or, if from the same species, is substantially modified from its original form by deliberate human intervention.

By “host cell” is meant a cell, which contains a vector and supports the replication and/or expression of the expression vector. Host cells may be prokaryotic cells such as E. coli, or eukaryotic cells such as yeast, insect, plant, amphibian, or mammalian cells. Preferably, host cells are monocotyledonous or dicotyledonous plant cells, including but not limited to maize, sorghum, sunflower, soybean, wheat, alfalfa, rice, cotton, canola, barley, millet and tomato. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The terms “isolated” refers to material, such as a nucleic acid or a protein, which is substantially or essentially free from components which normally accompany or interact with it as found in its naturally occurring environment. The isolated material optionally comprises material not found with the material in its natural environment. Nucleic acids, which are “isolated”, as defined herein, are also referred to as “heterologous” nucleic acids. Unless otherwise stated, the term “PDR nucleic acid” means a nucleic acid comprising a polynucleotide (“PDR polynucleotide”) encoding a PDR polypeptide.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules, which comprise and substantially represent the entire transcribed fraction of a genome of a specified organism. Construction of exemplary nucleic acid libraries, such as genomic and cDNA libraries, is taught in standard molecular biology references such as Berger and Kimmel, GUIDE TO MOLECULAR CLONING TECHNIQUES, from the series METHODS IN ENZYMOLOGY, vol. 152, Academic Press, Inc., San Diego, Calif. (1987); Sambrook, et al., MOLECULAR CLONING: A LABORATORY MANUAL, 2^{nd} ed., vols. 1-3 (1989); and CURRENT PROTOCOLS IN MOLECULAR BIOLOGY, Ausubel et al., eds, Current Protocols, a joint venture between Greene Publishing Associates, Inc. and John Wiley & Sons, Inc. (1994 Supplement).

As used herein “operably linked” includes reference to a functional linkage between a first sequence, such as a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds suspension cultures, embryos, meristematic regions, callus tissue, leaves, roots, shoots, gametophytes, sporophytes, pollen, and microspores. The class of plants, which can be used in the methods of the invention, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium, and Triticum. A particularly preferred plant is Zea mays.

As used herein, “yield” includes reference to bushels per acre of a grain crop at harvest, as adjusted for grain moisture (15% typically). Grain moisture is measured in the grain at harvest. The adjusted test weight of grain is determined to be the weight in pounds per bushel, adjusted for grain moisture level at harvest.

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide,” “peptide,” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses, and bacteria which comprise genes expressed in plant cells such Agrobacterium or Rhizobium. Examples are promoters that preferentially initiate transcription in certain tissues, such as leaves, roots, seeds, fibres, xylem vessels, tracheids, or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may effect transcription by inducible promoters include anaerobic conditions or the presence of light. Another type of promoter is a developmentally regulated promoter, for example, a promoter that drives expression during pollen development. Tissue preferred, cell type specific, developmentally regulated, and inducible promoters constitute the class of “non-constitutive” promoters. A “constitutive” promoter is a promoter, which is active under most environmental conditions.

The term “PDR polypeptide” refers to one or more amino acid sequences. The term is also inclusive of fragments, variants, homologs, alleles or precursors (e.g., preproproteins or proproteins) thereof. A “PDR protein” comprises a PDR polypeptide. Unless otherwise stated, the term “PDR nucleic acid” means a nucleic acid comprising a polynucleotide (“PDR polynucleotide”) encoding a PDR polypeptide.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The terms “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass known analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity, and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to at least equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-84 (1984): Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, LABORATORY TECHNIQUES IN BIOCHEMISTRY AND MOLECULAR BIOLOGY—HYBRIDIZATION WITH NUCLEIC ACID PROBES, part I, chapter 2, “Overview of principles of hybridization and the strategy of nucleic acid probe assays,” Elsevier, New York (1993); and CURRENT PROTOCOLS IN MOLECULAR BIOLOGY, chapter 2, Ausubel, et al., eds, Greene Publishing and Wiley-Interscience, New York (1995). Unless otherwise stated, in the present application, high stringency is defined as hybridization in 4×SSC, 5×Denhardt's (5 g Ficoll, 5 g polyvinypyrrolidone, 5 g bovine serum albumin in 500 ml of water), 0.1 mg/ml boiled salmon sperm DNA, and 25 mM Na phosphate at 65° C., and a wash in 0.1×SSC, 0.1% SDS at 65° C.

As used herein, “transgenic plant” includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides or polypeptides: (a) “reference sequence,” (b) “comparison window,” (c) “sequence identity,” (d) “percentage of sequence identity,” and (e) “substantial identity.”

As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

As used herein, “comparison window” means reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, Adv. Appl. Math 2:482 (1981), may conduct optimal alignment of sequences for comparison; by the homology alignment algorithm (GAP) of Needleman and Wunsch, J. Mol. Biol. 48:443-53 (1970); by the search for similarity method (Tfasta and Fasta) of Pearson and Lipman, Proc. Natl. Acad. Sci. USA 85:2444 (1988); by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Version 8 (available from Genetics Computer Group (GCG® programs (Accelrys, Inc., San Diego, Calif.)). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65, and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, CURRENT PROTOCOLS IN MOLECULAR BIOLOGY, Chapter 19, Ausubel, et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Clayerie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90%, and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

Another indication that nucleotide sequences are substantially identical is if two molecules hybridize to each other under stringent conditions. The degeneracy of the genetic code allows for many amino acids substitutions that lead to variety in the nucleotide sequence that code for the same amino acid, hence it is possible that the DNA sequence could code for the same polypeptide but not hybridize to each other under stringent conditions. This may occur, e.g., when a copy of a nucleic acid is created using the maximum codon degeneracy permitted by the genetic code. One indication that two nucleic acid sequences are substantially identical is that the polypeptide, which the first nucleic acid encodes, is immunologically cross reactive with the polypeptide encoded by the second nucleic acid.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm of Needleman and Wunsch, supra. An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides, which are “substantially similar” share sequences as, noted above except that residue positions, which are not identical, may differ by conservative amino acid changes.

The invention discloses PDR polynucleotides and polypeptides. The novel nucleotides and proteins of the invention have an expression pattern which indicates that they regulate cell development and thus play an important role in plant development. The polynucleotides are expressed in various plant tissues. The polynucleotides and polypeptides thus provide an opportunity to manipulate plant development to alter seed and vegetative tissue development, timing or composition. This may be used to create a sterile plant, a seedless plant or a plant with altered endosperm composition.

TABLE 1
Sequence Identification and nomenclature

| Sequence ID | | |
|---|---|---|
| numbers | | |
| (Polynucleotide, | | |
| polypeptide) | Current Name | Other nomenclature |
| 1, 2 | ZmPDR01 | ZmTFL1 |
| 3, 4 | ZmPDR02 | ZmTFL2 |
| 5, 6 | ZmPDR03 | ZmTFL3 |
| 7, 8 | ZmPDR04 | ZmTFL4 |
| 9, 10 | ZmPDR05 | ZmTFL5 |
| 11, 12 | ZmPDR06 | ZmTFL_C10 |
| 13, 14 | ZmPDR07 | ZmTFL_C04 |
| 15, 16 | ZmPDR08 | ZmTFL_C14 |
| 17, 18 | ZmPDR09 | ZmFT4 |
| 19, 20 | ZmPDR10 | ZmFT5 |
| 21, 22 | ZmPDR11 | ZmFT6 |
| 23, 24 | ZmPDR12 | ZmFT2 |
| 25, 26 | ZmPDR13 | ZmTFL_C05 |
| 27, 28 | ZmPDR14 | ZmFT1 |
| 29, 30 | ZmPDR15 | ZmFT3 |
| 31, 32 | ZmPDR16 | ZmFT7 |
| 33, 34 | ZmPDR17 | ZmTFL_C01 |
| 35, 36 | ZmPDR18 | ZmTFL_C02 |
| 37, 38 | ZmPDR19 | ZmTFL_C03 |
| 39, 40 | ZmPDR20 | ZmTFL_C06 |
| 41, 42 | ZmPDR21 | ZmTFL_C07 |
| 43, 44 | ZmPDR22 | ZmTFL_C08 |
| 45, 46 | ZmPDR23 | ZmTFL_C09 |
| 47, 48 | ZmPDR24 | ZmTFL_C11 |
| 49, 50 | ZmPDR25 | ZmTFL_C12 |
| 51, 52 | ZmPDR26 | ZmTFL_C13 |
| 53, 54 | ZmPDR27 | ZmTFL_C15 |
| 55, 56 | ZmPDR28 | ZmTFL_C19 |
| 57, 58 | OsPDR01 | OsTFL01 |
| 59, 60 | OsPDR02 | OsTFL02 |
| 61, 62 | OsPDR03 | OsTFL03 |
| 63, 64 | OsPDR04 | OsTFL04 |
| 65, 66 | OsPDR05 | OsTFL05 |
| 67, 68 | OsPDR06 | OsTFL06 |
| 69, 70 | OsPDR07 | OsTFL07 |
| 71, 72 | OsPDR08 | OsTFL08 |
| 73, 74 | OsPDR09 | OsTFL09 |
| 75, 76 | OsPDR10 | OsTFL10 |
| 77, 78 | OsPDR11 | OsTFL11 |
| 79, 80 | OsPDR12 | OsTFL12 |
| 81, 82 | OsPDR13 | OsTFL13 |
| 83, 84 | OsPDR14 | OsTFL14 |
| 85, 86 | OsPDR15 | OsTFL15 |
| 87, 88 | OsPDR16 | OsTFL16 |
| 89, 90 | OsPDR17 | OsTFL17 |
| 91, 92 | OsPDR18 | OsTFL18 |
| 93, 94 | OsPDR19 | OsTFL19 |
| 95, 96 | OsPDR20 | OsTFL20 |
| 97, 98 | OsPDR21 | OsTFL22 |
| 99, 100 | SbPDR01 | SbTFL_01 |
| 101, 102 | SbPDR02 | SbTFL_02 |
| 103, 104 | SbPDR03 | SbTFL_03 |
| 105, 106 | SbPDR04 | SbTFL_04 |
| 107, 108 | SbPDR05 | SbTFL_05 |
| 109, 110 | SbPDR06 | SbTFL_06 |
| 111, 112 | SbPDR07 | SbTFL_07 |
| 113, 114 | SbPDR08 | SbTFL_08 |
| 115, 116 | SbPDR09 | SbTFL_09 |
| 117, 118 | SbPDR10 | SbTFL_10 |
| 119, 120 | SbPDR11 | SbTFL_11 |
| 121, 122 | SbPDR12 | SbTFL_12 |
| 123, 124 | SbPDR13 | SbTFL_13 |
| 125, 126 | SbPDR14 | SbTFL_14 |
| 127, 128 | SbPDR15 | SbTFL_15 |
| 129, 130 | SbPDR16 | SbTFL_16 |
| 131, 132 | SbPDR17 | SbTFL_17 |
| 133, 134 | SbPDR18 | SbTFL_18 |
| 135, 136 | SbPDR19 | SbTFL_19 |
| 137, 138 | SbPDR20 | SbTFL_20 |
| 139, 140 | SbPDR21 | SbTFL_21 |
| 141, 142 | SbPDR22 | SbTFL_22 |
| 143, 144 | SbPDR23 | SbTFL_23 |
| 145, 146 | SbPDR24 | SbTFL_24 |
| 147, 148 | AcPDR01 | AcTFL_01 |
| 149, 150 | TaPDR01 | TaTFL_01 |
| 151, 152 | TaPDR02 | TaTFL_02 |
| 153, 154 | GmPDR01 | Gm_TFL_01 |
| 155, 156 | GmPDR02 | Gm_TFL_02 |
| 157, 158 | GmPDR03 | Gm_TFL_03 |
| 159, 160 | GmPDR04 | Gm_TFL_04 |
| 161, 162 | GmPDR05 | Gm_TFL_05 |
| 163, 164 | GmPDR06 | Gm_TFL_06 |
| 165, 166 | GmPDR07 | Gm_TFL_07 |
| 167, 168 | HaPDR01 | HaTFL_01 |
| 168, 170 | HaPDR02 | HaTFL_02 |
| 171, 172 | HaPDR03 | HaTFL_03 |
| 173, 174 | AtPDR01 | At_TFL1 |
| 175, 176 | AtPDR02 | At_CEN |
| 177, 178 | AtPDR03 | At_BFT |
| 179, 180 | AtPDR04 | At_FT |
| 181, 182 | AtPDR05 | At_TSF |
| 183, 184 | AtPDR06 | At_MFT |

### Nucleic Acids

The present invention provides, inter alia, isolated nucleic acids of RNA, DNA, and analogs and/or chimeras thereof, comprising a PDR polynucleotide.

The present invention also includes polynucleotides optimized for expression in different organisms. For example, for expression of the polynucleotide in a maize plant, the sequence can be altered to account for specific codon preferences and to alter GC content as according to Murray, et al, supra. Maize codon usage for 28 genes from maize plants is listed in Table 5 of Murray, et al., supra.

The PDR nucleic acids of the present invention comprise isolated PDR polynucleotides which are inclusive of:
(a) a polynucleotide encoding a PDR polypeptide and conservatively modified and polymorphic variants thereof;
(b) a polynucleotide having at least 70% sequence identity with polynucleotides of (a) or (b);
(c) complementary sequences of polynucleotides of (a) or (b).

### Construction of Nucleic Acids

The isolated nucleic acids of the present invention can be made using (a) standard recombinant methods, (b) synthetic techniques, or combinations thereof. In some embodiments, the polynucleotides of the present invention will be cloned, amplified, or otherwise constructed from a fungus or bacteria.

The nucleic acids may conveniently comprise sequences in addition to a polynucleotide of the present invention. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention. The nucleic acid of the present invention—excluding the polynucleotide sequence—is optionally a vector, adapter, or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present invention less the length of its polynucleotide of the present invention is less than 20 kilobase pairs, often less than 15 kb, and frequently less than 10 kb. Use of cloning vectors, expression vectors, adapters, and linkers is well known in the art. Exemplary nucleic acids include such vectors as: M13, lambda ZAP Express, lambda ZAP II, lambda gt10, lambda gt11, pBK-CMV, pBK-RSV, pBluescript II, lambda DASH II, lambda EMBL 3, lambda EMBL 4, pWE15, SuperCos 1, SurfZap, Uni-ZAP, pBC, pBS+/−, pSG5, pBK, pCR-Script, pET, pSPUTK, p3′SS, pGEM, pSK+/−, pGEX, pSPORTI and II, pOPRSVI CAT, pOPI3 CAT, pXT1, pSG5, pPbac, pMbac, pMC1neo, pOG44, pOG45, pFRTβGAL, pNEOβGAL, pRS403, pRS404, pRS405, pRS406, pRS413, pRS414, pRS415, pRS416, lambda MOSSIox, and lambda MOSEIox. Optional vectors for the present invention include but are not limited to, lambda ZAP II, and pGEX. For a description of various nucleic acids see, e.g., Stratagene Cloning Systems, Catalogs 1995, 1996, 1997 (La Jolla, Calif.); and, Amersham Life Sciences, Inc, Catalog '97 (Arlington Heights, Ill.).

### Synthetic Methods for Constructing Nucleic Acids

The isolated nucleic acids of the present invention can also be prepared by direct chemical synthesis by methods such as the phosphotriester method of Narang, et al., (1979) Meth. Enzymol. 68:90-9; the phosphodiester method of Brown, et al., (1979) Meth. Enzymol. 68:109-51; the diethylphosphoramidite method of Beaucage, et al., (1981) Tetra. Letts. 22(20):1859-62; the solid phase phosphoramidite triester method described by Beaucage, et al., supra, e.g., using an automated synthesizer, e.g., as described in Needham-VanDevanter, et al., (1984) Nucleic Acids Res. 12:6159-68; and, the solid support method of U.S. Pat. No. 4,458,066. Chemical synthesis generally produces a single stranded oligonucleotide. This may be converted into double stranded DNA by hybridization with a complementary sequence or by polymerization with a DNA polymerase using the single strand as a template. One of skill will recognize that while chemical synthesis of DNA is limited to sequences of about 100 bases, longer sequences may be obtained by the ligation of shorter sequences.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res.15:8125) and the 5<G>7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present invention provides 5′ and/or 3′ UTR regions for modulation of translation of heterologous coding sequences.

Further, the polypeptide-encoding segments of the polynucleotides of the present invention can be modified to alter codon usage. Altered codon usage can be employed to alter translational efficiency and/or to optimize the coding sequence for expression in a desired host or to optimize the codon usage in a heterologous sequence for expression in maize. Codon usage in the coding regions of the polynucleotides of the present invention can be analyzed statistically using commercially available software packages such as “Codon Preference” available from the University of Wisconsin Genetics Computer Group. See, Devereaux, et al., (1984) Nucleic Acids Res. 12:387-395); or MacVector 4.1 (Eastman Kodak Co., New Haven, Conn.). Thus, the present invention provides a codon usage frequency characteristic of the coding region of at least one of the polynucleotides of the present invention. The number of polynucleotides (3 nucleotides per amino acid) that can be used to determine a codon usage frequency can be any integer from 3 to the number of polynucleotides of the present invention as provided herein. Optionally, the polynucleotides will be full-length sequences. An exemplary number of sequences for statistical analysis can be at least 1, 5, 10, 20, 50 or 100.

### Sequence Shuffling

The present invention provides methods for sequence shuffling using polynucleotides of the present invention, and compositions resulting therefrom. Sequence shuffling is described in PCT Publication Number 96/19256. See also, Zhang, et al., (1997) Proc. Natl. Acad. Sci. USA 94:4504-9; and Zhao, et al., (1998) Nature Biotech 16:258-61. Generally, sequence shuffling provides a means for generating libraries of polynucleotides having a desired characteristic, which can be selected or screened for. Libraries of recombinant polynucleotides are generated from a population of related sequence polynucleotides, which comprise sequence regions, which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation, or other expression property of a gene or transgene, a replicative element, a protein-binding element, or the like, such as any feature which confers a selectable or detectable property. In some embodiments, the selected characteristic will be an altered Kₘ and/or K_{cat} over the wild-type protein as provided herein. In other embodiments, a protein or polynucleotide generated from sequence shuffling will have a ligand binding affinity greater than the non-shuffled wild-type polynucleotide. In yet other embodiments, a protein or polynucleotide generated from sequence shuffling will have an altered pH optimum as compared to the non-shuffled wild-type polynucleotide. The increase in such properties can be at least 110%, 120%, 130%, 140% or greater than 150% of the wild-type value.

### Recombinant Expression Cassettes

The present invention further provides recombinant expression cassettes comprising a nucleic acid of the present invention. A nucleic acid sequence coding for the desired polynucleotide of the present invention, for example a cDNA or a genomic sequence encoding a polypeptide long enough to code for an active protein of the present invention, can be used to construct a recombinant expression cassette which can be introduced into the desired host cell. A recombinant expression cassette will typically comprise a polynucleotide of the present invention operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

A plant promoter fragment can be employed which will direct expression of a polynucleotide of the present invention in all tissues of a regenerated plant. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, the 35S promoter from cauliflower mosaic virus (CaMV), as described in Odell, et al., (1985) Nature 313:810-2; rice actin (McElroy, et al., (1990) Plant Cell 163-171); ubiquitin (Christensen, et al., (1992) Plant Mol. Biol. 12:619-632 and Christensen, et al., (1992) Plant Mol. Biol. 18:675-89); pEMU (Last, et al., (1991) Theor. Appl. Genet. 81:581-8); MAS (Velten, et al., (1984) EMBO J. 3:2723-30); and maize H3 histone (Lepetit, et al., (1992) Mol. Gen. Genet. 231:276-85; and Atanassvoa, et al., (1992) Plant Journal 2(3):291-300); ALS promoter, as described in PCT Application Number WO 96/30530; and other transcription initiation regions from various plant genes known to those of skill. For the present invention ubiquitin is the preferred promoter for expression in monocot plants.

Alternatively, the plant promoter can direct expression of a polynucleotide of the present invention in a specific tissue or may be otherwise under more precise environmental or developmental control. Such promoters are referred to here as “inducible” promoters. Environmental conditions that may effect transcription by inducible promoters include pathogen attack, anaerobic conditions, or the presence of light. Examples of inducible promoters are the Adh1 promoter, which is inducible by hypoxia or cold stress, the Hsp70 promoter, which is inducible by heat stress, and the PPDK promoter, which is inducible by light.

Examples of promoters under developmental control include promoters that initiate transcription only, or preferentially, in certain tissues, such as leaves, roots, fruit, seeds, or flowers. The operation of a promoter may also vary depending on its location in the genome. Thus, an inducible promoter may become fully or partially constitutive in certain locations.

If polypeptide expression is desired, it is generally desirable to include a polyadenylation region at the 3′-end of a polynucleotide coding region. The polyadenylation region can be derived from a variety of plant genes, or from T-DNA. The 3′ end sequence to be added can be derived from, for example, the nopaline synthase or octopine synthase genes, or alternatively from another plant gene, or less preferably from any other eukaryotic gene. Examples of such regulatory elements include, but are not limited to, 3′ termination and/or polyadenylation regions such as those of the Agrobacterium tumefaciens nopaline synthase (nos) gene (Bevan, et al., (1983) Nucleic Acids Res. 12:369-85); the potato proteinase inhibitor II (PINII) gene (Keil, et al., (1986) Nucleic Acids Res. 14:5641-50; and An, et al., (1989) Plant Cell 1:115-22); and the CaMV 19S gene (Mogen, et al., (1990) Plant Cell 2:1261-72).

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, (1988) Mol. Cell. Biol. 8:4395-4405; Callis, et al., (1987) Genes Dev. 1:1183-200). Such intron enhancement of gene expression is typically greatest when placed near the 5′ end of the transcription unit. Use of maize introns Adh1-S intron 1, 2, and 6, the Bronze-1 intron are known in the art. See generally, THE MAIZE HANDBOOK, Chapter 116, Freeling and Walbot, eds., Springer, New York (1994).

Plant signal sequences, including, but not limited to, signal-peptide encoding DNA/RNA sequences which target proteins to the extracellular matrix of the plant cell (Dratewka-Kos, et al., (1989) J. Biol. Chem. 264:4896-900), such as the Nicotiana plumbaginifolia extension gene (DeLoose, et al., (1991) Gene 99:95-100); signal peptides which target proteins to the vacuole, such as the sweet potato sporamin gene (Matsuka, et al., (1991) Proc. Natl. Acad. Sci. USA 88:834) and the barley lectin gene (Wilkins, et al., (1990) Plant Cell, 2:301-13); signal peptides which cause proteins to be secreted, such as that of PRIb (Lind, et al., (1992) Plant Mol. Biol. 18:47-53) or the barley alpha amylase (BAA) (Rahmatullah, et al., (1989) Plant Mol. Biol. 12:119, and hereby incorporated by reference), or signal peptides which target proteins to the plastids such as that of rapeseed enoyl-Acp reductase (Verwaert, et al., (1994) Plant Mol. Biol. 26:189-202) are useful in the invention. The barley alpha amylase signal sequence fused to the PDR polynucleotide is the preferred construct for expression in maize for the present invention.

The vector comprising the sequences from a polynucleotide of the present invention will typically comprise a marker gene, which confers a selectable phenotype on plant cells. Usually, the selectable marker gene will encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance, genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene), or other such genes known in the art. The bar gene encodes resistance to the herbicide basta, and the ALS gene encodes resistance to the herbicide chlorsulfuron.

Typical vectors useful for expression of genes in higher plants are well known in the art and include vectors derived from the tumor-inducing (Ti) plasmid of Agrobacterium tumefaciens described by Rogers, et al., (1987) Meth. Enzymol. 153:253-77. These vectors are plant integrating vectors in that on transformation, the vectors integrate a portion of vector DNA into the genome of the host plant. Exemplary A. tumefaciens vectors useful herein are plasmids pKYLX6 and pKYLX7 of Schardl, et al., (1987) Gene 61:1-11, and Berger, et al., (1989) Proc. Natl. Acad. Sci. USA, 86:8402-6. Another useful vector herein is plasmid pBI101.2 that is available from CLONTECH Laboratories, Inc. (Palo Alto, Calif.).

### Expression of Proteins in Host Cells

Using the nucleic acids of the present invention, one may express a protein of the present invention in a recombinantly engineered cell such as bacteria, yeast, insect, mammalian, or preferably plant cells. The cells produce the protein in a non-natural condition (e.g., in quantity, composition, location, and/or time), because they have been genetically altered through human intervention to do so.

It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

In brief summary, the expression of isolated nucleic acids encoding a protein of the present invention will typically be achieved by operably linking, for example, the DNA or cDNA to a promoter (which is either constitutive or inducible), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present invention. To obtain high level expression of a cloned gene, it is desirable to construct expression vectors which contain, at the minimum, a strong promoter, such as ubiquitin, to direct transcription, a ribosome binding site for translational initiation, and a transcription/translation terminator. Constitutive promoters are classified as providing for a range of constitutive expression. Thus, some are weak constitutive promoters, and others are strong constitutive promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a “strong promoter” drives expression of a coding sequence at a “high level,” or about 1/10 transcripts to about 1/100 transcripts to about 1/1,000 transcripts.

One of skill would recognize that modifications could be made to a protein of the present invention without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

### Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli is also useful. Examples of such markers include genes specifying resistance to ampicillin, tetracycline, or chloramphenicol.

The vector is selected to allow introduction of the gene of interest into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva, et al., (1983) Gene 22:229-35; Mosbach, et al., (1983) Nature 302:543-5). The pGEX-4T-1 plasmid vector from Pharmacia is the preferred E. coli expression vector for the present invention.

### Expression in Eukaryotes

A variety of eukaryotic expression systems such as yeast, insect cell lines, plant and mammalian cells, are known to those of skill in the art. As explained briefly below, the present invention can be expressed in these eukaryotic systems. In some embodiments, transformed/transfected plant cells, as discussed infra, are employed as expression systems for production of the proteins of the instant invention.

Synthesis of heterologous proteins in yeast is well known. Sherman, et al., METHODS IN YEAST GENETICS, Cold Spring Harbor Laboratory (1982) is a well recognized work describing the various methods available to produce the protein in yeast. Two widely utilized yeasts for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

A protein of the present invention, once expressed, can be isolated from yeast by lysing the cells and applying standard protein isolation techniques to the lysates or the pellets. The monitoring of the purification process can be accomplished by using Western blot techniques or radioimmunoassay of other standard immunoassay techniques.

The sequences encoding proteins of the present invention can also be ligated to various expression vectors for use in transfecting cell cultures of, for instance, mammalian, insect, or plant origin. Mammalian cell systems often will be in the form of monolayers of cells although mammalian cell suspensions may also be used. A number of suitable host cell lines capable of expressing intact proteins have been developed in the art, and include the HEK293, BHK21, and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen, et al., (1986) Immunol. Rev. 89:49), and necessary processing information sites, such as ribosome binding sites, RNA splice sites, polyadenylation sites (e.g., an SV40 large T Ag poly A addition site), and transcriptional terminator sequences. Other animal cells useful for production of proteins of the present invention are available, for instance, from the American Type Culture Collection Catalogue of Cell Lines and Hybridomas (7^{th} ed., 1992).

Appropriate vectors for expressing proteins of the present invention in insect cells are usually derived from the SF9 baculovirus. Suitable insect cell lines include mosquito larvae, silkworm, armyworm, moth, and Drosophila cell lines such as a Schneider cell line (see, e.g., Schneider, (1987) J. Embryol. Exp. Morphol. 27:353-65).

As with yeast, when higher animal or plant host cells are employed, polyadenlyation or transcription terminator sequences are typically incorporated into the vector. An example of a terminator sequence is the polyadenlyation sequence from the bovine growth hormone gene. Sequences for accurate splicing of the transcript may also be included. An example of a splicing sequence is the VP1 intron from SV40 (Sprague, et al., (1983) J. Virol. 45:773-81). Additionally, gene sequences to control replication in the host cell may be incorporated into the vector such as those found in bovine papilloma virus type-vectors (Saveria-Campo, “Bovine Papilloma Virus DNA a Eukaryotic Cloning Vector,” in DNA CLONING: A PRACTICAL APPROACH, vol. II, Glover, ed., IRL Press, Arlington, Va., pp. 213-38 (1985)).

In addition, the gene for PDR placed in the appropriate plant expression vector can be used to transform plant cells. The polypeptide can then be isolated from plant callus or the transformed cells can be used to regenerate transgenic plants. Such transgenic plants can be harvested, and the appropriate tissues (seed or leaves, for example) can be subjected to large scale protein extraction and purification techniques.

### Plant Transformation Methods

Numerous methods for introducing foreign genes into plants are known and can be used to insert a PDR polynucleotide into a plant host, including biological and physical plant transformation protocols. See, e.g., Miki, et al., “Procedure for Introducing Foreign DNA into Plants,” in METHODS IN PLANT MOLECULAR BIOLOGY AND BIOTECHNOLOGY, Glick and Thompson, eds., CRC Press, Inc., Boca Raton, pp. 67-88 (1993). The methods chosen vary with the host plant, and include chemical transfection methods such as calcium phosphate, microorganism-mediated gene transfer such as Agrobacterium (Horsch, et al., (1985) Science 227:1229-31), electroporation, micro-injection, and biolistic bombardment.

Expression cassettes and vectors and in vitro culture methods for plant cell or tissue transformation and regeneration of plants are known and available. See, e.g., Gruber, et al., “Vectors for Plant Transformation,” in METHODS IN PLANT MOLECULAR BIOLOGY AND BIOTECHNOLOGY, supra, pp. 89-119.

The isolated polynucleotides or polypeptides may be introduced into the plant by one or more techniques typically used for direct delivery into cells. Such protocols may vary depending on the type of organism, cell, plant, or plant cell, i.e., monocot or dicot, targeted for gene modification. Suitable methods of transforming plant cells include microinjection (Crossway, et al., (1986) Biotechniques 4:320-334; and U.S. Pat. No. 6,300,543), electroporation (Riggs, et al., (1986) Proc. Natl. Acad. Sci. USA 83:5602-5606, direct gene transfer (Paszkowski, et al., (1984) EMBO J. 3:2717-2722), and ballistic particle acceleration (see, for example, Sanford, et al., U.S. Pat. No. 4,945,050; WO 91/10725; and McCabe, et al., (1988) Biotechnology 6:923-926). Also see, Tomes, et al., Direct DNA Transfer into Intact Plant Cells Via Microprojectile Bombardment pp. 197-213 in Plant Cell, Tissue and Organ Culture, Fundamental Methods eds. O. L. Gamborg & G. C. Phillips. Springer-Verlag Berlin Heidelberg New York, 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 91/10725 (maize); Klein, et al., (1988) Plant Physiol. 91:440-444 (maize); Fromm, et al., (1990) Biotechnology 8:833-839; and Gordon-Kamm, et al., (1990) Plant Cell 2:603-618 (maize); Hooydaas-Van Slogteren and Hooykaas (1984) Nature (London) 311:763-764; Bytebier, et al., (1987) Proc. Natl. Acad. Sci. USA 84:5345-5349 (Liliaceae); De Wet, et al., (1985) In The Experimental Manipulation of Ovule Tissues, ed. Chapman, et al., pp. 197-209. Longman, N.Y. (pollen); Kaeppler, et al., (1990) Plant Cell Reports 9:415-418; and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255; and Christou and Ford (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185); all of which are herein incorporated by reference.

### Agrobacterium-Mediated Transformation

The most widely utilized method for introducing an expression vector into plants is based on the natural transformation system of Agrobacterium. A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra; and Moloney, et al., (1989) Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion, or via a binary system where the vir gene is present on a separate vector. Such systems, vectors for use therein, and methods of transforming plant cells are described in U.S. Pat. No. 4,658,082; U.S. Pat. No. 913,914, filed Oct. 1, 1986, as referenced in U.S. Pat. No. 5,262,306, issued Nov. 16, 1993; and Simpson, et al., (1986) Plant Mol. Biol. 6:403-15 (also referenced in the '306 patent); all incorporated by reference in their entirety.

Once constructed, these plasmids can be placed into A. rhizogenes or A. tumefaciens and these vectors used to transform cells of plant species, which are ordinarily susceptible to Fusarium or Alternaria infection. Several other transgenic plants are also contemplated by the present invention including but not limited to soybean, corn, sorghum, alfalfa, rice, clover, cabbage, banana, coffee, celery, tobacco, cowpea, cotton, melon and pepper. The selection of either A. tumefaciens or A. rhizogenes will depend on the plant being transformed thereby. In general A. tumefaciens is the preferred organism for transformation. Most dicotyledonous plants, some gymnosperms, and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Compositae, and Chenopodiaceae. Monocot plants can now be transformed with some success. European Patent Application Number 604 662 A1 discloses a method for transforming monocots using Agrobacterium. European Application Number 672 752 A1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors, and cultured under conditions, which promote plant regeneration. Roots or shoots transformed by inoculation of plant tissue with A. rhizogenes or A. tumefaciens, containing the gene coding for the fumonisin degradation enzyme, can be used as a source of plant tissue to regenerate fumonisin-resistant transgenic plants, either via somatic embryogenesis or organogenesis. Examples of such methods for regenerating plant tissue are disclosed in Shahin, (1985) Theor. Appl. Genet. 69:235-40; U.S. Pat. No. 4,658,082; Simpson, et al., supra; and U.S. Pat. Nos. 913,913 and 913,914, both filed Oct. 1, 1986, as referenced in U.S. Pat. No. 5,262,306, issued Nov. 16, 1993, the entire disclosures therein incorporated herein by reference.

### Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei, et al., (1994) The Plant Journal 6:271-82). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206; and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731; and Christou, et al., (1987) Proc. Natl. Acad. Sci. USA 84:3962. Direct uptake of DNA into protoplasts using CaCl₂ precipitation, polyvinyl alcohol, or poly-L-ornithine has also been reported. See, e.g., Hain, et al., (1985) Mol. Gen. Genet. 199:161; and Draper, et al., (1982) Plant Cell Physiol. 23:451.

Electroporation of protoplasts and whole cells and tissues has also been described. See, e.g., Donn, et al., (1990) in Abstracts of the VIIth Int'l. Congress on Plant Cell and Tissue Culture IAPTC, A2-38, p. 53; D'Halluin, et al., (1992) Plant Cell 4:1495-505; and Spencer, et al., (1994) Plant Mol. Biol. 24:51-61.

Increasing the Activity and/or Level of a PDR Polypeptide

Methods are provided to increase the activity and/or level of the PDR polypeptide of the invention. An increase in the level and/or activity of the PDR polypeptide of the invention can be achieved by providing to the plant a PDR polypeptide. The PDR polypeptide can be provided by introducing the amino acid sequence encoding the PDR polypeptide into the plant, introducing into the plant a nucleotide sequence encoding a PDR polypeptide or alternatively by modifying a genomic locus encoding the PDR polypeptide of the invention.

As discussed elsewhere herein, many methods are known the art for providing a polypeptide to a plant including, but not limited to, direct introduction of the polypeptide into the plant, introducing into the plant (transiently or stably) a polynucleotide construct encoding a polypeptide having cell development regulator activity. It is also recognized that the methods of the invention may employ a polynucleotide that is not capable of directing, in the transformed plant, the expression of a protein or an RNA. Thus, the level and/or activity of a PDR polypeptide may be increased by altering the gene encoding the PDR polypeptide or its promoter. See, e.g., Kmiec, U.S. Pat. No. 5,565,350; Zarling, et al., PCT/US93/03868. Therefore mutagenized plants that carry mutations in PDR genes, where the mutations increase expression of the PDR gene or increase the cell development regulator activity of the encoded PDR polypeptide are provided.

Reducing the Activity and/or Level of a PDR Polypeptide

Methods are provided to reduce or eliminate the activity of a PDR polypeptide of the invention by transforming a plant cell with an expression cassette that expresses a polynucleotide that inhibits the expression of the PDR polypeptide. The polynucleotide may inhibit the expression of the PDR polypeptide directly, by preventing translation of the PDR messenger RNA, or indirectly, by encoding a polypeptide that inhibits the transcription or translation of a PDR gene encoding a PDR polypeptide. Methods for inhibiting or eliminating the expression of a gene in a plant are well known in the art, and any such method may be used in the present invention to inhibit the expression of a PDR polypeptide.

In accordance with the present invention, the expression of a PDR polypeptide is inhibited if the protein level of the PDR polypeptide is less than 70% of the protein level of the same PDR polypeptide in a plant that has not been genetically modified or mutagenized to inhibit the expression of that PDR polypeptide. In particular embodiments of the invention, the protein level of the PDR polypeptide in a modified plant according to the invention is less than 60%, less than 50%, less than 40%, less than 30%, less than 20%, less than 10%, less than 5%, or less than 2% of the protein level of the same PDR polypeptide in a plant that is not a mutant or that has not been genetically modified to inhibit the expression of that PDR polypeptide. The expression level of the PDR polypeptide may be measured directly, for example, by assaying for the level of PDR polypeptide expressed in the plant cell or plant, or indirectly, for example, by measuring the cell development regulator activity of the PDR polypeptide in the plant cell or plant, or by measuring the cell development in the plant. Methods for performing such assays are described elsewhere herein.

In other embodiments of the invention, the activity of the PDR polypeptides is reduced or eliminated by transforming a plant cell with an expression cassette comprising a polynucleotide encoding a polypeptide that inhibits the activity of a PDR polypeptide. The cell development regulator activity of a PDR polypeptide is inhibited according to the present invention if the cell development regulator activity of the PDR polypeptide is less than 70% of the cell development regulator activity of the same PDR polypeptide in a plant that has not been modified to inhibit the cell development regulator activity of that PDR polypeptide. In particular embodiments of the invention, the cell development regulator activity of the PDR polypeptide in a modified plant according to the invention is less than 60%, less than 50%, less than 40%, less than 30%, less than 20%, less than 10% or less than 5% of the cell development regulator activity of the same PDR polypeptide in a plant that that has not been modified to inhibit the expression of that PDR polypeptide. The cell development regulator activity of a PDR polypeptide is “eliminated” according to the invention when it is not detectable by the assay methods described elsewhere herein. Methods of determining the cell development regulator activity of a PDR polypeptide are described elsewhere herein.

In other embodiments, the activity of a PDR polypeptide may be reduced or eliminated by disrupting the gene encoding the PDR polypeptide. The invention encompasses mutagenized plants that carry mutations in PDR genes, where the mutations reduce expression of the PDR gene or inhibit the cell development regulator activity of the encoded PDR polypeptide.

Thus, many methods may be used to reduce or eliminate the activity of a PDR polypeptide. In addition, more than one method may be used to reduce the activity of a single PDR polypeptide. Non-limiting examples of methods of reducing or eliminating the expression of PDR polypeptides are given below.

### 1. Polynucleotide-Based Methods:

In some embodiments of the present invention, a plant is transformed with an expression cassette that is capable of expressing a polynucleotide that inhibits the expression of a PDR polypeptide of the invention. The term “expression” as used herein refers to the biosynthesis of a gene product, including the transcription and/or translation of said gene product. For example, for the purposes of the present invention, an expression cassette capable of expressing a polynucleotide that inhibits the expression of at least one PDR polypeptide is an expression cassette capable of producing an RNA molecule that inhibits the transcription and/or translation of at least one PDR polypeptide of the invention. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of a PDR polypeptide are given below.

i. Sense Suppression/Cosuppression

In some embodiments of the invention, inhibition of the expression of a PDR polypeptide may be obtained by sense suppression or cosuppression. For cosuppression, an expression cassette is designed to express an RNA molecule corresponding to all or part of a messenger RNA encoding a PDR polypeptide in the “sense” orientation. Over expression of the RNA molecule can result in reduced expression of the native gene. Accordingly, multiple plant lines transformed with the cosuppression expression cassette are screened to identify those that show the greatest inhibition of PDR polypeptide expression.

The polynucleotide used for cosuppression may correspond to all or part of the sequence encoding the PDR polypeptide, all or part of the 5′ and/or 3′ untranslated region of a PDR polypeptide transcript, or all or part of both the coding sequence and the untranslated regions of a transcript encoding a PDR polypeptide. In some embodiments where the polynucleotide comprises all or part of the coding region for the PDR polypeptide, the expression cassette is designed to eliminate the start codon of the polynucleotide so that no protein product will be translated.

Cosuppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763; and U.S. Pat. Nos. 5,034,323; 5,283,184 and 5,942,657; each of which is herein incorporated by reference. The efficiency of cosuppression may be increased by including a poly-dT region in the expression cassette at a position 3′ to the sense sequence and 5′ of the polyadenylation signal. See, US Patent Application Publication Number 20020048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323; herein incorporated by reference.

ii. Antisense Suppression

In some embodiments of the invention, inhibition of the expression of the PDR polypeptide may be obtained by antisense suppression. For antisense suppression, the expression cassette is designed to express an RNA molecule complementary to all or part of a messenger RNA encoding the PDR polypeptide. Over expression of the antisense RNA molecule can result in reduced expression of the native gene. Accordingly, multiple plant lines transformed with the antisense suppression expression cassette are screened to identify those that show the greatest inhibition of PDR polypeptide expression.

The polynucleotide for use in antisense suppression may correspond to all or part of the complement of the sequence encoding the PDR polypeptide, all or part of the complement of the 5′ and/or 3′ untranslated region of the PDR transcript, or all or part of the complement of both the coding sequence and the untranslated regions of a transcript encoding the PDR polypeptide. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550 or greater may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, US Patent Application Publication Number 20020048814, herein incorporated by reference.

iii. Double-Stranded RNA Interference

In some embodiments of the invention, inhibition of the expression of a PDR polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of PDR polypeptide expression. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743, and WO 99/49029, WO 99/53050, WO 99/61631, and WO 00/49035; each of which is herein incorporated by reference.

iv. Hairpin RNA Interference and Intron-Containing Hairpin RNA Interference

In some embodiments of the invention, inhibition of the expression of one or a PDR polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz (2000) Proc. Natl. Acad. Sci. USA 97:4985-4990; Stoutjesdijk, et al., (2002) Plant Physiol. 129:1723-1731; and Waterhouse and Helliwell, (2003) Nat. Rev. Genet. 4:29-38. Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini, et al., BMC Biotechnology 3:7, and US Patent Application Publication Number 20030175965; each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295, and US Patent Application Publication Number 20030180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 02/00904, herein incorporated by reference.

### V. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the PDR polypeptide). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362, and U.S. Pat. No. 6,646,805, each of which is herein incorporated by reference.

vi. Ribozymes

In some embodiments, the polynucleotide expressed by the expression cassette of the invention is catalytic RNA or has ribozyme activity specific for the messenger RNA of the PDR polypeptide. Thus, the polynucleotide causes the degradation of the endogenous messenger RNA, resulting in reduced expression of the PDR polypeptide. This method is described, for example, in U.S. Pat. No. 4,987,071, herein incorporated by reference.

vii. Small Interfering RNA or Micro RNA

In some embodiments of the invention, inhibition of the expression of a PDR polypeptide may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example, Javier, et al., (2003) Nature 425:257-263, herein incorporated by reference.

For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of PDR expression, the 22-nucleotide sequence is selected from a PDR transcript sequence and contains 22 nucleotides of said PDR sequence in sense orientation and 21 nucleotides of a corresponding antisense sequence that is complementary to the sense sequence. miRNA molecules are highly efficient at inhibiting the expression of endogenous genes, and the RNA interference they induce is inherited by subsequent generations of plants.

### 2. Polypeptide-Based Inhibition of Gene Expression

In one embodiment, the polynucleotide encodes a zinc finger protein that binds to a gene encoding a PDR polypeptide, resulting in reduced expression of the gene. In particular embodiments, the zinc finger protein binds to a regulatory region of a PDR gene. In other embodiments, the zinc finger protein binds to a messenger RNA encoding a PDR polypeptide and prevents its translation. Methods of selecting sites for targeting by zinc finger proteins have been described, for example, in U.S. Pat. No. 6,453,242, and methods for using zinc finger proteins to inhibit the expression of genes in plants are described, for example, in US Patent Application Publication Number 20030037355; each of which is herein incorporated by reference.

### 3. Polypeptide-Based Inhibition of Protein Activity

In some embodiments of the invention, the polynucleotide encodes an antibody that binds to at least one PDR polypeptide, and reduces the cell development regulator activity of the PDR polypeptide. In another embodiment, the binding of the antibody results in increased turnover of the antibody-PDR complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

### 4. Gene Disruption

In some embodiments of the present invention, the activity of a PDR polypeptide is reduced or eliminated by disrupting the gene encoding the PDR polypeptide. The gene encoding the PDR polypeptide may be disrupted by any method known in the art. For example, in one embodiment, the gene is disrupted by transposon tagging. In another embodiment, the gene is disrupted by mutagenizing plants using random or targeted mutagenesis, and selecting for plants that have reduced cell development regulator activity.

i. Transposon Tagging

In one embodiment of the invention, transposon tagging is used to reduce or eliminate the PDR activity of one or more PDR polypeptide. Transposon tagging comprises inserting a transposon within an endogenous PDR gene to reduce or eliminate expression of the PDR polypeptide. “PDR gene” is intended to mean the gene that encodes a PDR polypeptide according to the invention.

In this embodiment, the expression of one or more PDR polypeptide is reduced or eliminated by inserting a transposon within a regulatory region or coding region of the gene encoding the PDR polypeptide. A transposon that is within an exon, intron, 5′ or 3′ untranslated sequence, a promoter, or any other regulatory sequence of a PDR gene may be used to reduce or eliminate the expression and/or activity of the encoded PDR polypeptide.

Methods for the transposon tagging of specific genes in plants are well known in the art. See, for example, Maes, et al., (1999) Trends Plant Sci. 4:90-96; Dharmapuri and Sonti, (1999) FEMS Microbiol. Lett. 179:53-59; Meissner, et al. (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928). In addition, the TUSC process for selecting Mu insertions in selected genes has been described in Bensen, et al., (1995) Plant Cell 7:75-84; Mena, et al., (1996) Science 274:1537-1540; and U.S. Pat. No. 5,962,764; each of which is herein incorporated by reference.

ii. Mutant Plants with Reduced Activity

Additional methods for decreasing or eliminating the expression of endogenous genes in plants are also known in the art and can be similarly applied to the instant invention. These methods include other forms of mutagenesis, such as ethyl methanesulfonate-induced mutagenesis, deletion mutagenesis, and fast neutron deletion mutagenesis used in a reverse genetics sense (with PCR) to identify plant lines in which the endogenous gene has been deleted. For examples of these methods see, Ohshima, et al., (1998) Virology 243:472-481; Okubara, et al., (1994) Genetics 137:867-874; and Quesada, et al., (2000) Genetics 154:421-436; each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant invention. See, McCallum, et al., (2000) Nat. Biotechnol. 18:455-457, herein incorporated by reference.

Mutations that impact gene expression or that interfere with the function (cell development regulator activity) of the encoded protein are well known in the art. Insertional mutations in gene exons usually result in null-mutants. Mutations in conserved residues are particularly effective in inhibiting the cell development regulator activity of the encoded protein. Conserved residues of plant PDR polypeptides suitable for mutagenesis with the goal to eliminate cell development regulator activity have been described. Such mutants can be isolated according to well-known procedures, and mutations in different PDR loci can be stacked by genetic crossing. See, for example, Gruis, et al., (2002) Plant Cell 14:2863-2882.

In another embodiment of this invention, dominant mutants can be used to trigger RNA silencing due to gene inversion and recombination of a duplicated gene locus. See, for example, Kusaba, et al., (2003) Plant Cell 15:1455-1467.

The invention encompasses additional methods for reducing or eliminating the activity of one or more PDR polypeptides. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such vectors and methods of use are known in the art. See, for example, U.S. Pat. Nos. 5,565,350; 5,731,181; 5,756,325; 5,760,012; 5,795,972 and 5,871,984; each of which are herein incorporated by reference. See also, WO 98/49350, WO 99/07865, WO 99/25821 and Beetham, et al., (1999) Proc. Natl. Acad. Sci. USA 96:8774-8778; each of which is herein incorporated by reference.

iii. Modulating Plant Architecture

In specific methods, the increased growth of yield improvement associated tissues in a plant is caused by increasing the level or activity of the PDR polypeptide in the plant. Methods for increasing the level and/or activity of PDR polypeptides in a plant are discussed elsewhere herein. Briefly, such methods comprise providing a PDR polypeptide of the invention to a plant and thereby increasing the level and/or activity of the PDR polypeptide. In other embodiments, a PDR nucleotide sequence encoding a PDR polypeptide can be provided by introducing into the plant a polynucleotide comprising a PDR nucleotide sequence of the invention, expressing the PDR sequence, increasing the activity of the PDR polypeptide, and thereby causing increases in the yield improvement associate related plant architecture in the plant or plant part. In other embodiments, the PDR nucleotide construct introduced into the plant is stably incorporated into the genome of the plant.

In other methods, the number and shape of a yield associated plant tissue is increased by increasing the level and/or activity of the PDR polypeptide in the plant. Such methods are disclosed in detail elsewhere herein. In one such method, a PDR nucleotide sequence is introduced into the plant and expression of said PDR nucleotide sequence increases the activity of the PDR polypeptide, and thereby increasing the size or shape of the tissue in the plant or plant part. In other embodiments, the PDR nucleotide construct introduced into the plant is stably incorporated into the genome of the plant.

As discussed above, one of skill will recognize the appropriate promoter to use to modulate the level/activity of the yield improvement associated polypeptides in the plant. Exemplary promoters for this embodiment have been disclosed elsewhere herein.

Accordingly, the present invention further provides plants having modified plant architecture when compared to the architecture of a control plant. In one embodiment, maize plants of the invention have an increased level/activity of the PDR polypeptide of the invention and thus exhibit one or more of the following phenotypic characteristics: an increased kernel number per ear, increased spikelet density, tassel branch number, pollen production, improved canopy shape and increased photosynthetic capacity in the leaf tissue, and improved stalk strength and plant standability. In other embodiments, the plants of the invention have an increased level of the PDR polypeptide of the invention resulting in an alteration of vascular bundle structure and number in the plant tissue. In other embodiments, such plants have stably incorporated into their genome a nucleic acid molecule comprising a PDR nucleotide sequence of the invention operably linked to a promoter that drives expression in the plant cell.

iv. Modulating Root Development

Methods for modulating root development in a plant are provided. By “modulating root development” is intended any alteration in the development of the plant root when compared to a control plant. Such alterations in root development include, but are not limited to, alterations in the growth rate of the primary root, the fresh root weight, the extent of lateral and adventitious root formation, the vasculature system, meristem development, or radial expansion.

Methods for modulating root development in a plant are provided. The methods comprise modulating the level and/or activity of the PDR polypeptide in the plant. In one method, a PDR sequence of the invention is provided to the plant. In another method, the PDR nucleotide sequence is provided by introducing into the plant a polynucleotide comprising a PDR nucleotide sequence of the invention, expressing the PDR sequence, and thereby modifying root development. In still other methods, the PDR nucleotide construct introduced into the plant is stably incorporated into the genome of the plant.

In other methods, root development is modulated by altering the level or activity of the PDR polypeptide in the plant. An increase in PDR activity can result in at least one or more of the following alterations to root development, including, but not limited to, larger root meristems, increases in root growth, enhanced radial expansion, an enhanced vasculature system, increased root branching, more adventitious roots, and/or an increase in fresh root weight when compared to a control plant.

As used herein, “root growth” encompasses all aspects of growth of the different parts that make up the root system at different stages of its development in both monocotyledonous and dicotyledonous plants. It is to be understood that enhanced root growth can result from enhanced growth of one or more of its parts including the primary root, lateral roots, adventitious roots, etc.

Methods of measuring such developmental alterations in the root system are known in the art. See, for example, US Patent Application Publication Number 2003/0074698 and Werner, et al., (2001) PNAS 18:10487-10492, both of which are herein incorporated by reference.

As discussed above, one of skill will recognize the appropriate promoter to use to modulate root development in the plant. Exemplary promoters for this embodiment include constitutive promoters and root-preferred promoters. Exemplary root-preferred promoters have been disclosed elsewhere herein.

Stimulating root growth and increasing root mass by increasing the activity and/or level of the PDR polypeptide also finds use in improving the standability of a plant. The term “resistance to lodging” or “standability” refers to the ability of a plant to fix itself to the soil. For plants with an erect or semi-erect growth habit, this term also refers to the ability to maintain an upright position under adverse (environmental) conditions. This trait relates to the size, depth and morphology of the root system. In addition, stimulating root growth and increasing root mass by decreasing the level and/or activity of the PDR polypeptide also finds use in promoting in vitro propagation of explants.

Furthermore, higher root biomass production due to an increased level and/or activity of PDR activity has a direct effect on the yield and an indirect effect of production of compounds produced by root cells or transgenic root cells or cell cultures of said transgenic root cells. One example of an interesting compound produced in root cultures is shikonin, the yield of which can be advantageously enhanced by said methods.

Accordingly, the present invention further provides plants having modulated root development when compared to the root development of a control plant. In some embodiments, the plant of the invention has an increased level/activity of the PDR polypeptide of the invention and has enhanced root growth and/or root biomass. In other embodiments, such plants have stably incorporated into their genome a nucleic acid molecule comprising a PDR nucleotide sequence of the invention operably linked to a promoter that drives expression in the plant cell.

v. Modulating Shoot and Leaf Development

Methods are also provided for modulating shoot and leaf development in a plant. By “modulating shoot and/or leaf development” is intended any alteration in the development of the plant shoot and/or leaf. Such alterations in shoot and/or leaf development include, but are not limited to, alterations in shoot meristem development, in leaf number, leaf size, leaf and stem vasculature, internode length, and leaf senescence. As used herein, “leaf development” and “shoot development” encompasses all aspects of growth of the different parts that make up the leaf system and the shoot system, respectively, at different stages of their development, both in monocotyledonous and dicotyledonous plants. Methods for measuring such developmental alterations in the shoot and leaf system are known in the art. See, for example, Werner, et al., (2001) PNAS 98:10487-10492 and US Patent Application Publication Number 2003/0074698, each of which is herein incorporated by reference.

The method for modulating shoot and/or leaf development in a plant comprises modulating the activity and/or level of a PDR polypeptide of the invention. In one embodiment, a PDR sequence of the invention is provided. In other embodiments, the PDR nucleotide sequence can be provided by introducing into the plant a polynucleotide comprising a PDR nucleotide sequence of the invention, expressing the PDR sequence, and thereby modifying shoot and/or leaf development. In other embodiments, the PDR nucleotide construct introduced into the plant is stably incorporated into the genome of the plant.

In specific embodiments, shoot or leaf development is modulated by increasing the level and/or activity of the PDR polypeptide in the plant. An increase in PDR activity can result in at least one or more of the following alterations in shoot and/or leaf development, including, but not limited to, increased leaf number, increased leaf surface, increased vascularity, longer internodes and improved growth, and altered leaf senescence, when compared to a control plant.

As discussed above, one of skill will recognize the appropriate promoter to use to modulate shoot and leaf development of the plant. Exemplary promoters for this embodiment include constitutive promoters, shoot-preferred promoters, shoot meristem-preferred promoters, and leaf-preferred promoters. Exemplary promoters have been disclosed elsewhere herein.

Decreasing PDR activity and/or level in a plant results in shorter internodes and stunted growth. Thus, the methods of the invention find use in producing dwarf plants. In addition, as discussed above, modulation PDR activity in the plant modulates both root and shoot growth. Thus, the present invention further provides methods for altering the root/shoot ratio. Shoot or leaf development can further be modulated by decreasing the level and/or activity of the PDR polypeptide in the plant.

Accordingly, the present invention further provides plants having modulated shoot and/or leaf development when compared to a control plant. In some embodiments, the plant of the invention has an increased level/activity of the PDR polypeptide of the invention. In other embodiments, the plant of the invention has a decreased level/activity of the PDR polypeptide of the invention.

vi. Modulating Reproductive Tissue Development

Methods for modulating reproductive tissue development are provided. In one embodiment, methods are provided to modulate floral development in a plant. By “modulating floral development” is intended any alteration in a structure of a plant's reproductive tissue as compared to a control plant in which the activity or level of the PDR polypeptide has not been modulated. “Modulating floral development” further includes any alteration in the timing of the development of a plant's reproductive tissue (i.e., a delayed or an accelerated timing of floral development) when compared to a control plant in which the activity or level of the PDR polypeptide has not been modulated. Macroscopic alterations may include changes in size, shape, number, or location of reproductive organs, the developmental time period that these structures form, or the ability to maintain or proceed through the flowering process in times of environmental stress. Microscopic alterations may include changes to the types or shapes of cells that make up the reproductive organs.

The method for modulating floral development in a plant comprises modulating PDR activity in a plant. In one method, a PDR sequence of the invention is provided. A PDR nucleotide sequence can be provided by introducing into the plant a polynucleotide comprising a PDR nucleotide sequence of the invention, expressing the PDR sequence, and thereby modifying floral development. In other embodiments, the PDR nucleotide construct introduced into the plant is stably incorporated into the genome of the plant.

In specific methods, floral development is modulated by increasing the level or activity of the PDR polypeptide in the plant. An increase in PDR activity can result in at least one or more of the following alterations in floral development, including, but not limited to, more rapid flowering, increased number of flowers, and increased seed set, when compared to a control plant. Inducing more rapid flowering can be used to enhance yield in forage crops such as alfalfa. Methods for measuring such developmental alterations in floral development are known in the art. See, for example, Mouradov, et al., (2002) The Plant Cell S111-S130, herein incorporated by reference.

As discussed above, one of skill will recognize the appropriate promoter to use to modulate floral development of the plant. Exemplary promoters for this embodiment include constitutive promoters, inducible promoters, shoot-preferred promoters, and inflorescence-preferred promoters.

In other methods, floral development is modulated by increasing the level and/or activity of the PDR sequence of the invention. Such methods can comprise introducing a PDR nucleotide sequence into the plant and increasing the activity of the PDR polypeptide. In other methods, the PDR nucleotide construct introduced into the plant is stably incorporated into the genome of the plant. Increased expression of the PDR sequence of the invention can modulate floral development during periods of stress. Such methods are described elsewhere herein. Accordingly, the present invention further provides plants having modulated floral development when compared to the floral development of a control plant. Compositions include plants having a decreased level/activity of the PDR polypeptide of the invention and having an altered floral development. Compositions also include plants having an increased level/activity of the PDR polypeptide of the invention wherein the plant maintains or proceeds through the flowering process in times of stress.

Methods are also provided for the use of the PDR sequences of the invention to increase seed number. The method comprises increasing the activity of the PDR sequences in a plant or plant part, such as the seed. An increase in seed size and/or number comprises an increased size or number of the seed and/or an increase in the size of one or more seed part including, for example, the embryo, endosperm, seed coat, aleurone, or cotyledon.

As discussed above, one of skill will recognize the appropriate promoter to use to increase seed size and/or seed number. Exemplary promoters of this embodiment include constitutive promoters, inducible promoters, seed-preferred promoters, embryo-preferred promoters, and endosperm-preferred promoters.

Accordingly, the present invention further provides plants having an increased seed weight and/or seed number when compared to a control plant. In other embodiments, plants having an increased vigor and plant yield are also provided. In some embodiments, the plant of the invention has an increased level/activity of the PDR polypeptide of the invention and has an increased seed number and/or seed size. In other embodiments, such plants have stably incorporated into their genome a nucleic acid molecule comprising a PDR nucleotide sequence of the invention operably linked to a promoter that drives expression in the plant cell.

vii. Method of Use for PDR Promoter Polynucleotides

The polynucleotides comprising the PDR promoters disclosed in the present invention, as well as variants and fragments thereof, are useful in the genetic manipulation of any host cell, preferably plant cell, when assembled with a DNA construct such that the promoter sequence is operably linked to a nucleotide sequence comprising a polynucleotide of interest. In this manner, the PDR promoter polynucleotides of the invention are provided in expression cassettes along with a polynucleotide sequence of interest for expression in the host cell of interest. As discussed in Example 2 below, the PDR promoter sequences of the invention are expressed in a variety of tissues and thus the promoter sequences can find use in regulating the temporal and/or the spatial expression of polynucleotides of interest.

Synthetic hybrid promoter regions are known in the art. Such regions comprise upstream promoter elements of one polynucleotide operably linked to the promoter element of another polynucleotide. In an embodiment of the invention, heterologous sequence expression is controlled by a synthetic hybrid promoter comprising the PDR promoter sequences of the invention, or a variant or fragment thereof, operably linked to upstream promoter element(s) from a heterologous promoter. Upstream promoter elements that are involved in the plant defense system have been identified and may be used to generate a synthetic promoter. See, for example, Rushton, et al., (1998) Curr. Opin. Plant Biol. 1:311-315. Alternatively, a synthetic PDR promoter sequence may comprise duplications of the upstream promoter elements found within the PDR promoter sequences.

It is recognized that the promoter sequence of the invention may be used with its native PDR coding sequences. A DNA construct comprising the PDR promoter operably linked with its native PDR gene may be used to transform any plant of interest to bring about a desired phenotypic change, such as modulating cell development, modulating root, shoot, leaf, floral, and embryo development, stress tolerance, and any other phenotype described elsewhere herein.

The promoter nucleotide sequences and methods disclosed herein are useful in regulating expression of any heterologous nucleotide sequence in a host plant in order to vary the phenotype of a plant. Various changes in phenotype are of interest including modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism as well as those affecting kernel size, sucrose loading, and the like.

In certain embodiments the nucleic acid sequences of the present invention can be used in combination (“stacked”) with other polynucleotide sequences of interest in order to create plants with a desired phenotype. The combinations generated can include multiple copies of any one or more of the polynucleotides of interest. The polynucleotides of the present invention may be stacked with any gene or combination of genes to produce plants with a variety of desired trait combinations, including but not limited to traits desirable for animal feed such as high oil genes (e.g., U.S. Pat. No. 6,232,529); balanced amino acids (e.g., hordothionins (U.S. Pat. Nos. 5,990,389; 5,885,801; 5,885,802 and 5,703,409); barley high lysine (Williamson, et al., (1987) Eur. J. Biochem. 165:99-106; and WO 98/20122); and high methionine proteins (Pedersen, et al., (1986) J. Biol. Chem. 261:6279; Kirihara, et al., (1988) Gene 71:359; and Musumura, et al., (1989) Plant Mol. Biol. 12:123)); increased digestibility (e.g., modified storage proteins (U.S. patent application Ser. No. 10/053,410, filed Nov. 7, 2001); and thioredoxins (U.S. patent application Ser. No. 10/005,429, filed Dec. 3, 2001)), the disclosures of which are herein incorporated by reference. The polynucleotides of the present invention can also be stacked with traits desirable for insect, disease or herbicide resistance (e.g., Bacillus thuringiensis toxic proteins (U.S. Pat. Nos. 5,366,892; 5,747,450; 5,737,514; 5723,756; 5,593,881; Geiser, et al., (1986) Gene 48:109); lectins (Van Damme, et al., (1994) Plant Mol. Biol. 24:825); fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE) and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present invention with polynucleotides affecting agronomic traits such as male sterility (e.g., see, U.S. Pat. No. 5,583,210), stalk strength, flowering time, or transformation technology traits such as cell cycle regulation or gene targeting (e.g., WO 99/61619; WO 00/17364; WO 99/25821), the disclosures of which are herein incorporated by reference.

In one embodiment, sequences of interest improve plant growth and/or crop yields. For example, sequences of interest include agronomically important genes that result in improved primary or lateral root systems. Such genes include, but are not limited to, nutrient/water transporters and growth induces. Examples of such genes, include but are not limited to, maize plasma membrane H⁺-ATPase (MHA2) (Frias, et al., (1996) Plant Cell 8:1533-44); AKT1, a component of the potassium uptake apparatus in Arabidopsis, (Spalding, et al., (1999) J Gen Physiol 113:909-18); RML genes which activate cell division cycle in the root apical cells (Cheng, et al., (1995) Plant Physiol 108:881); maize glutamine synthetase genes (Sukanya, et al., (1994) Plant Mol Biol 26:1935-46) and hemoglobin (Duff, et al., (1997) J. Biol. Chem. 27:16749-16752, Arredondo-Peter, et al., (1997) Plant Physiol. 115:1259-1266; Arredondo-Peter, et al., (1997) Plant Physiol 114:493-500 and references sited therein). The sequence of interest may also be useful in expressing antisense nucleotide sequences of genes that that negatively affects root development.

Additionally, agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801; 5,885,802 and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson, et al., (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. patent application Ser. No. 08/740,682, filed Nov. 1, 1996, and WO 98/20133, the disclosures of which are herein incorporated by reference. Other proteins include methionine-rich plant proteins such as from sunflower seed (Lilley, et al., (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen, et al., (1986) J. Biol. Chem. 261:6279; Kirihara, et al., (1988) Gene 71:359; both of which are herein incorporated by reference); and rice (Musumura, et al., (1989) Plant Mol. Biol. 12:123, herein incorporated by reference). Other agronomically important genes encode latex, Floury 2, growth factors, seed storage factors, and transcription factors.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881 and Geiser, et al., (1986) Gene 48:109); and the like.

Genes encoding disease resistance traits include detoxification genes, such as against fumonosin (U.S. Pat. No. 5,792,931); avirulence (avr) and disease resistance (R) genes (Jones, et al., (1994) Science 266:789; Martin, et al., (1993) Science 262:1432; and Mindrinos, et al., (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049; 5,885,801; 5,885,802 and 5,990,389.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see, Schubert, et al., (1988) J. Bacteriol. 170:5837-5847) facilitate expression of polyhyroxyalkanoates (PHAs).

Exogenous products include plant enzymes and products as well as those from other sources including procaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

This invention can be better understood by reference to the following non-limiting examples. It will be appreciated by those skilled in the art that other embodiments of the invention may be practiced without departing from the spirit and the scope of the invention as herein disclosed and claimed.

### EXAMPLES

### Example 1

### Enhancement of Multiple Agronomic Traits in ZmPDR1 Transgenic Plants

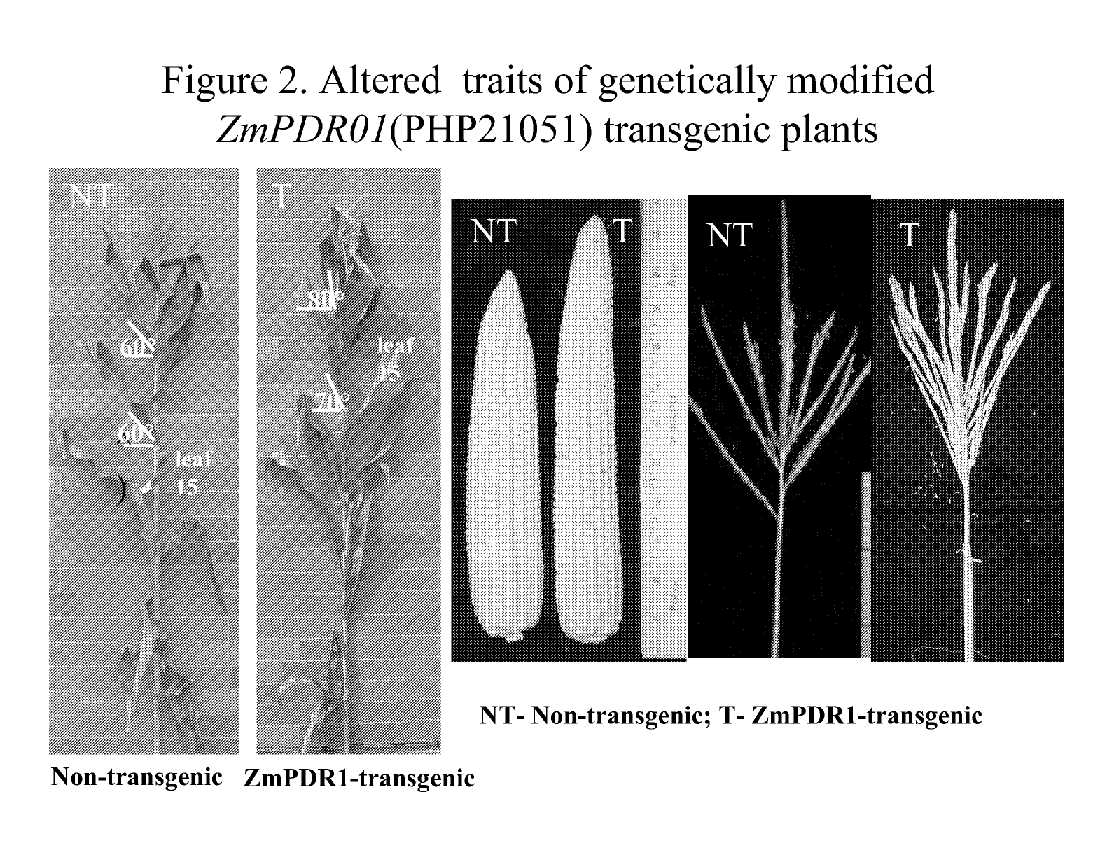

Eight maize ESTs were previously identified in the Pioneer/Dupont EST database by homology to CETS proteins (PCT patent application, publication number WO02044390). Maize ESTs p0104.cabak14rb (ZmPDR01) and p0118.chsaq04rb (ZmPDR02) were integrated into a transcriptional cassette between the Ubiquitin promoter and PINII terminator in a standard vector for Agrobacterium transformation. 25 events were generated for each construct (PHP21051, UBI::ZmPDR01 and PHP21836, UBI:: ZmPDR02). In a greenhouse T0 plants exhibited extended vegetative growth, produced more and larger leaves. In addition, transgenic plants produced tassels with increased spikelet density and increased amount of pollen. The ectopic expression of ZmPDR01/02 in the study demonstrated a complex phenotype with altered vegetative and reproductive characteristics. (FIG. 2)

Transgenic (T1) seeds were harvested from 14 events of ZmPDR01 (PHP21051) in a greenhouse and were planted in the field in summer 2004. Under the field conditions, transgenic plants continued to show unique characteristic differences in structure compared to non-transgenic siblings. (FIG. 2) Transgenic plants showed a distinct canopy shape with upright wide leaves. They produced a tassel with high spikelet density with copious pollen shed. Transgenic plants had also elongated ears; an advantageous trait for yield enhancement.

The following traits were measured in T1 UBI::ZmPDR01 plants at maturity: leaf number, leaf angle, leaf area, internode length, stalk strength, spikelet number and spikelet density per main tassel branch, spikelet number per ear row, and ear row numbers. The average morphometric results are shown in Table 2. Statistical analysis of all traits measured was performed with MINITAB® Statistical Software, MINITAB® (Release 14.12.0). Results are shown in tables 2-8 below. Twelve of the 14 transgenic events showed positive changes in multiple agronomic traits in comparison with non-transgenic siblings: more and larger upright leaves, more spikelets per tassel, copious pollen, more spikelet number per ear, and stronger stalks.

TABLE 2
Average morphometric results for T1 UBI::ZMPDR01 plants

| | Non- | | |
|---|---|---|---|
| Trait | transgenic | Transgenic | Transgenic/NT |
| Leaf number | 20.9 | 22.1 | 1-2 leaves more |
| Leaf angle* | 61.7° | 72.7° | 10.4° increase |
| Leaf area, cm²* | 300 | 363 | 21% increase |
| Internodes length, cm | 171.2 | 165.5 | Not significant |
| Stalk strength, by force | 55.5 | 71.0 | 30% increase |
| (kg) to break | | | |
| Spikelets per main | 214 | 55.513 | 78% increase |
| tassel branch | | | |
| Spikelets per ear row | 46 | 56 | 22% increase |
| Row number per ear | 14-16 | 16 | No differences |

*4 top leaves

### Ear Traits

The ZmPDR01 transgenic plants show up to a 20% increase in spikelet numbers per row. T-test confirmed that differences between the transgenic and non-transgenic spikelet number per row are statistically significant (Table 3A). The row number was not changed in transgenic ears. The estimated total number of spikelets per ear is shown in a Table 3B. The transgenic ears produced up to 24% more kernels compared to non-transgenics. Grain yield in corn is highly correlated with kernel number per ear. Therefore, the ZmPDR01 transgene may enhance yield potential by increasing total kernel number on mature ears. The transgene-induced phenotypes would therefore show dominant gain of function trait and have a good penetrance in hybrids and increased grain yield in various backgrounds.

TABLE 3
Statistical analysis of a spikelet number
from transgenic and non-transgenic ears.
A. Paired T-Test and CI: Spikelets per
Ear Row Non-transgenic, Transgenic
Paired T for Non-transgenic - Transgenic

| | N | Mean | StDev | SE Mean |
|---|---|---|---|---|
| Non-transgenic | 12 | 45.0208 | 7.7221 | 2.2292 |
| Transgenic | 12 | 56.0417 | 3.1277 | 0.9029 |
| Difference | 12 | −11.0208 | 9.3319 | 2.6939 |

95% CI for mean difference: (−16.9501, −5.0916)
T-Test of mean difference = 0 (vs not = 0): T-Value = −4.09
P-Value = 0.002

B. Paired T-Test and CI: Total Number of Spikelets
per ears Non-transgenic, Transgenic
Paired T for Estimated Non-transgenic - Transgenic

| | N | Mean | StDev | SE Mean |
|---|---|---|---|---|
| Non-transgenic | 12 | 705.104 | 143.969 | 41.560 |
| Transgenic | 12 | 876.083 | 104.092 | 30.049 |
| Difference | 12 | −170.979 | 151.040 | 43.602 |

95% CI for mean difference: (−266.946, −75.013)
T-Test of mean difference = 0 (vs not = 0): T-Value = −3.92
P-Value = 0.002

### Tassel Traits

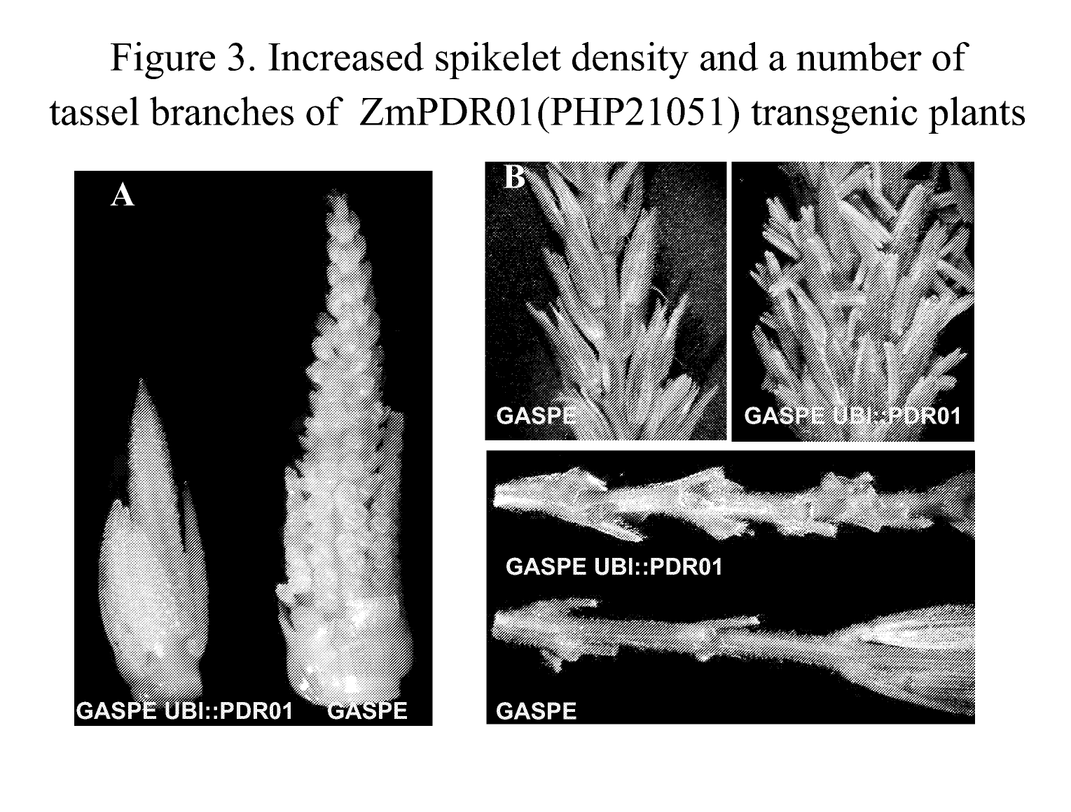

Transgenes ZmPDR01 and ZmPDR02, driven by Ubiquitin promoter changed tassel morphology with respect to the number of lateral branches (FIG. 3A) and spikelet density (3B). This phenotype has a strong penetrance in different transformable lines including GS3 and Gaspe flint. The number of lateral branches in Gaspe Flint background is increased at least 3 times, from 3-4 in Gaspe up to 20 in transgenics (FIG. 3A). The most prominent feature of transgenic tassels is an increased spikelet density, which is most evident on the central rachis (spike). Side by side comparison (FIG. 3B) of the central spikes in control Gaspe and transgenic Gaspe UBI:: ZmPDR01 plants revealed that the distance between adjacent whorls of rachillas in transgenic Gaspe UBI:: ZmPDR01 is nearly half that found in control plants (FIG. 3B). Gaspe UBI:: ZmPDR01 tassel inflorescence meristems produce about two times more SPMs (spikelet pair meristems) per unit length than control GASPE plants.

T-test was performed to compare the transgenic and non-transgenic spikelet number per tassels. It confirmed that differences are statistically significant (Table 4).

TABLE 4
Statistical analysis of spikelet number from
transgenic and non-transgenic tassels
Paired T-Test and CI: Tassel Spikelet,
Tassel Spikelets per main branch
Paired T for Tassel Spikelet NonTransgenics -
Tassel Spikelets Transgenic

| | N | Mean | StDev | SE Mean |
|---|---|---|---|---|
| Tassel Spikelet | 11 | 210.364 | 45.840 | 13.821 |
| Tassel Spikelets | 11 | 351.379 | 84.151 | 25.372 |
| Difference | 11 | −141.015 | 98.156 | 29.595 |

95% CI for mean difference: (−206.958, −75.073)
T-Test of mean difference = 0 (vs not = 0): T-Value = −4.76
P-Value = 0.001

Paired T-Test and CI: Spikelet Density,
Spikelet Density per cm of length
Paired T for Spikelet Density NonTransgenics -
Spikelet Density Transgenics

| | N | Mean | StDev | SE Mean |
|---|---|---|---|---|
| Spikelet Density | 11 | 7.9491 | 1.4408 | 0.4344 |
| Spikelet Density | 11 | 11.9973 | 1.8509 | 0.5581 |
| Difference | 11 | −4.04818 | 2.19032 | 0.66041 |

95% CI for mean difference: (−5.51966, −2.57670)
T-Test of mean difference = 0 (vs not = 0): T-Value = −6.13
P-Value = 0.000

Plants with a large tassel having high spikelet density and copious amount of pollen, as seen in ZmPDR01 transgenics, will improve hybrid seed production. Many hybrid seed production fields are planted in a 4:1 row pattern in which 4 rows are planted with the seed-bearing parent (female) and 1 row is planted with the pollen-bearing parent (male). Only the female rows are harvested for seed, thus only 80% of the land area is harvested. Seed companies must pay the grower for 100% of the acres under production. A large, prolific tassel such as that produced by over-expression of the ZmPDR01 gene would increase the percent of acres used to grow the female parent, thereby decreasing the total number of acres necessary for production. In addition, the large, highly branched tassel will release pollen over a longer period, thereby increasing the pollen shed window. This would improve seed set and reduce the risk of adventitious presence in seed production fields. Many of the best male inbreds have small tassels with a limited period of pollen shed. This requires delayed plantings or other expensive methods to extend the pollen-shed period. Moreover, many superior yielding inbred combinations are never used because male plants do not efficiently “nick” with the female. Incorporating ZmPDR01 into male inbreds would solve this problem by producing males that shed pollen for an extended period of time.

### Leaf Traits

The size, shape and number of leaves are important components of efficient light interception affecting photosynthetic capacity of the plants. The leaf area in ZmPDR01 transgenics plants above the ear increased about 20% over non-transgenic control plants. Those leaves are responsible for fixing 70-80% of the carbohydrates that ultimately end up in the ear. The ZmPDR01 transgenics have enhanced potential for both the source (more photosynthesis) and sink (more kernels to fill) sources as a means of increasing yield.

T-test was performed to compare the transgenic and non-transgenic leaf areas for 4 top leaves. The analysis confirmed that differences are statistically significant (Table 5).

TABLE 5
Statistical analysis of the leaf area from 4 top leaves for transgenic
and non-transgenic plants.
Paired T-Test and CI: Leaf Area Non-transgenic, Leaf Area Transgenic
Paired T for Leaf Area Non-transgenic - Leaf Area Transgenic

| | N | Mean | StDev | SE Mean |
|---|---|---|---|---|
| Leaf Area Non-tr | 12 | 300.948 | 53.077 | 15.322 |
| Leaf Area Transg | 12 | 362.798 | 57.735 | 16.667 |
| Difference | 12 | −61.8508 | 83.4149 | 24.0798 |

95% CI for mean difference: (−114.8502, −8.8515)
T-Test of mean difference = 0 (vs not = 0): T-Value = −2.57 P-Value = 0.026

The ZmPDR01 transgenics also exhibited upright top leaves (Table 6). Increases in the upright leaf habit is a trait that has been positively associated with hybrid yielding ability during the many years of hybrid selection (Duvick, 1992). This trait may result from breeding for high density planting and/or from more rigid stalks that increased rigidity of leaf mid-ribs causing a more upright leaf habit (Duvick, 1992). This allows for increased density in planting crops. Also, when coupled with increased pollen shed, the yield per acre production fields would increase. A statistical T-test was performed to compare the transgenic and non-transgenic leaf angles for 4 top leaves. It confirmed that the leaf inclination differences are statistically significant (Table 6).

TABLE 6
Statistical analysis of the leaf angles for 4 top leaves from transgenic
and non-transgenic plants.
Paired T-Test and CI: Leaf Angle NonTransgenic, Leaf Angle Transgenic
Paired for Leaf Angle NonTransgenic - Leaf Angle Transgenic

| | N | Mean | StDev | SE Mean |
|---|---|---|---|---|
| Leaf Angle NonTr | 12 | 7.19250 | 0.46014 | 0.13283 |
| Leaf Angle Trans | 12 | 7.88167 | 0.34604 | 0.09989 |
| Difference | 12 | −0.689167 | 0.629523 | 0.181728 |

95% CI for mean difference: (−1.089147, −0.289187)
T-Test of mean difference = 0 (vs not = 0): T-Value = −3.79 P-Value = 0.003

Transgenic plants also produced 1-2 leaves more than non-transgenic plants (Table 7), indicating that ZmPDR01 delayed the meristem transition from vegetative to reproductive phase. The delay is less than that found in Arabidopsis TFL1 expressing lines (Ratcliffe. et al., 1998).

TABLE 7
Statistical analysis of the leaf numbers from
transgenic and non-transgenic plants.
Paired T-Test and CI: Leaf Number Non Transgenic,
Leaf Number Transgenic
Paired T for Leaf Number Non Transgenic -
Leaf Number Transgenic

| | N | Mean | StDev | SE Mean |
|---|---|---|---|---|
| Leaf Number Non | 12 | 20.8650 | 1.0095 | 0.2914 |
| Leaf Number Tran | 12 | 22.1433 | 0.7640 | 0.2205 |
| Difference | 12 | −1.27833 | 1.18876 | 0.34317 |

95% CI for mean difference: (−2.03364, −0.52303)

### Stalk Traits

Stalk strength was tested by an Instron, model 4411 (Instron Corporation, 100 Royall Street, Canton, Mass. 02021), which measures force required to break stalks and also measures the stalk bend before breaking (Appenzeller, et al., (2004) Cellulose 11:287-299). Stalks were sampled from mature plants in a field, fully dried at room temperature and measurements were taken for two internodes below the ear. Stalk diameters, flexibility and strength were measured.

TABLE 8
Stalk strength measured by force (kg) to break a stalk

| | | | Standard | Standard |
|---|---|---|---|---|
| | Count | Mean for load(kg) | deviation | error |
| Transgenic | 19 | 70.971 | 21.866 | 5.016 |
| Non-transgenic | 15 | 55.513 | 15.479 | 3.997 |

TABLE 9
Stalk flexibility measured by displacement (mm)

| | | | Standard | Standard |
|---|---|---|---|---|
| | Count | Mean (mm) | deviation | error |
| Transgenic | 19 | .236 | .071 | .016 |
| Non-transgenic | 15 | .305 | .062 | .016 |

TABLE 10
Stalk diameter (mm)

| | | | Standard | Standard |
|---|---|---|---|---|
| | Count | Mean (mm) | deviation | error |
| Transgenic | 19 | 22.305 | 2.606 | .598 |
| Non-transgenic | 15 | 21.707 | 2.219 | .573 |

Maximum force to break a stalk was significantly different between transgenic and non-transgenic plants (Table 8). Transgenic stalks are up to 29% stronger than non-transgenic stalks. Stalk flexibility, measured as the stalk bend before breaking, statistically was not different (Table 9).

Intrernode diameters differed slightly between transgenics and non-transgenics (Table 10) therefore, the observed strength difference may not be due to merely a bigger stalk, a thicker rind, or some dry matter composition. One explanation could be differences in mechanical properties of the transgenic stalks, which are modified as the result of increased number or strength of vascular bundles. The increased stalk strength is a valuable agronomic trait, which reduces stalk lodging under certain environmental conditions. This trait is associated with improved standability and increased harvestable yield.

Morphometric analysis of vascular bundles in internodes was performed in control Gaspe plants and transgenic GASPE UBI::ZmPDR01 plants. Cross-sections were photographed under UV illumination to visualize cellular composition of stem tissues and vascular bundles based on auto-fluorescence. The number of vascular bundles was increased on average by a factor 1.28; the area of vascular bundles was increased by a factor of 1.43; and the area of metaxylem vessels was increased by a factor of 1.96, in ZmPDR01 transgenics compared with non-transgenic GASPE siblings. The ZmPDR01 gene affects vascular bundles in multiple ways by increasing overall numbers of vascular bundles, as well as their size. The analysis of bundles and vessels show the vascular bundles thickness increasing and themetaxylem vessels have a larger diameter.

This data is consistent with the in situ hybridization which shows that the ZmPDR01 gene is expressed in the vascular bundles. ZmPDR01 protein expressed under the ubiquitin promoter initiates more vascular bundles (or bigger bundles) in transgenic plants. Increased bundle strength could explain both the upright leaf habit and stronger stalks in transgenic plants. A more developed vasculature enhances a flux of nutrients across the plants and improves the overall plant vigor.

### Example 2

### Three-Dimensional Structure of ZMPDR01 and ZMPDR14 Proteins Suggest their Function as Kinase Effectors/Regulators

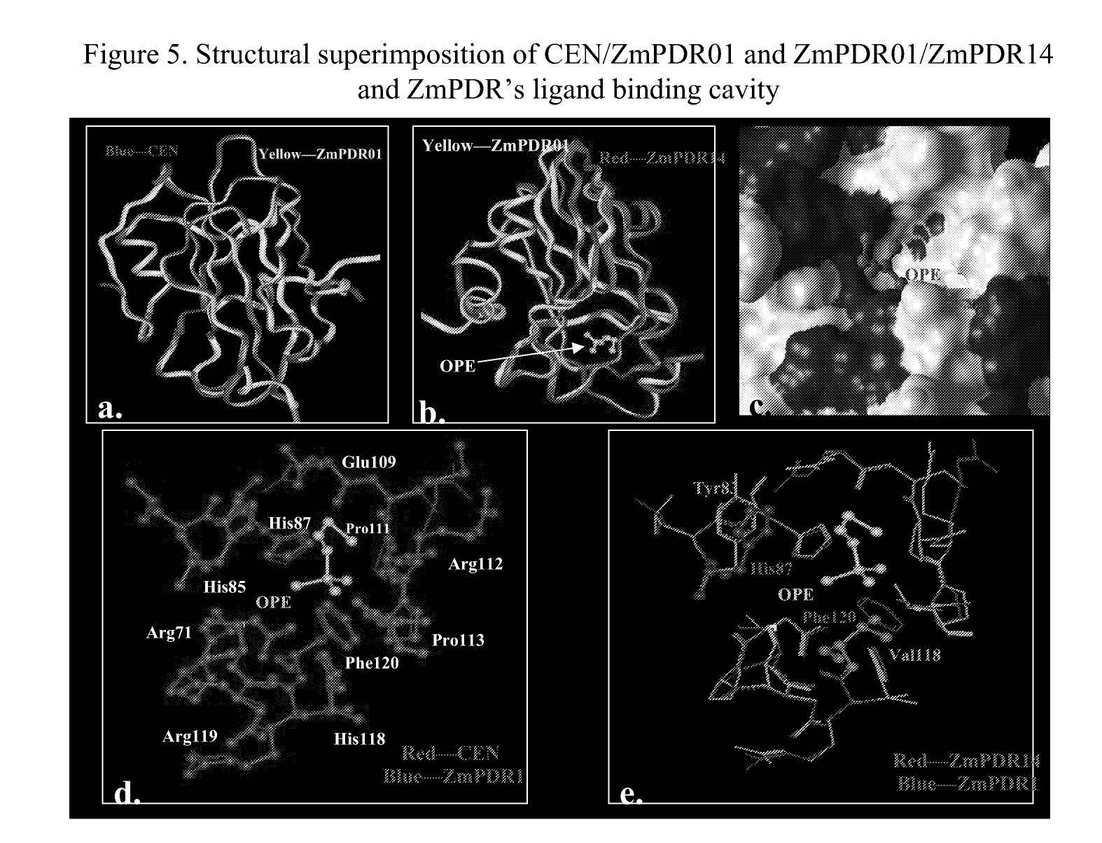

In order to predict biochemical function of maize PDR proteins, ZMPDR01 and ZMPDR14 (ESTs p0104.cabak14rb and cbn10.pk0052.f5 as described in PCT application WO02044390) were chosen for modeling. The crystallographic structure comparison demonstrated that the general fold and the anion binding-site are extremely well conserved among mammal, plant, and bacterial RKIP proteins (Banfield and Brady, 2000; Odabaei, et al., 2004). Similar to those experimentally determined structures, the ZmPDR01 and ZMPDR14 models each have the signature fold and the strongly conserved anion recognition pocket (FIG. 5a, b, d). The structural similarity at both ligand binding site and overall fold indicates the biological functions of ZmPDR01 and ZMPDR14, like other RKIP members, stem from the ability to form complexes with phosphorylated ligand, hence interfering with protein kinases and/or their effectors.

The ZmPDR01/ZMPDR14 structures were modeled with MODELERE (Sali and Blundell, 1993), an Insight II package for structural modeling. Two protein structures, PDB:1qou (Berman, et al., 2000) of Antirrhinum CENTRORADIALIS protein and PDB: 1b7a of the phosphatidylethanolamine-binding protein from bovine, were used as templates. The template structures were first structurally aligned together and then the maize sequences were aligned to the structures with a structure-based sequence alignment tool, in which the structural information was mainly captured in the position specific substitution score matrix and gap-penalty. The atom coordinates of modeling protein were assigned based on template, and subsequently underwent an energy minimization procedure to remove the bad contacts. The overall structures of ZMPDR01/ZMPDR14 are similar to the folding of other the RKIP members characterized as two anti-parallel β-sheets and long stranded-connecting loops (FIG. 5b). Superposition of either ZMPDR01 or ZmPDR14 to template structures gives an r.m.s.d (root mean square of deviation) of <1 Å.

The most striking feature among the RKIP family is the high conservation of the ligand binding-site. Extensive mutagenesis data especially from the mammalian and yeast proteins showed that the binding site and its surrounding region are crucial for protein activities. To identify the binding site in the modeled structures, a ligand surrogate OPE was mapped, phosphoric acid mono-(2-amino-ethyl), into the structural frame based on the overall structural alignment between the bovine RKIP complexed with OPE and ZmPDR1/ZMPDR14. After transformation, OPE fell in a well-defined binding pocket of modeled structures and its phosphate group formed an extensive hydrogen-bond network with the recognition residues (FIG. 5b, c, d, e). The comparison between CEN and ZmPDR01 revealed the near consensus of the major residues contributing the construction of the binding sites, including Asn71, His85, His87, Glu109, Pro111, Arg112, Pro113, His118, and Phe120 as in ZmPDR01 (FIG. 5d). However, the ZMPDR's anion recognition site is slightly different from that of ZmPDR01 or CEN. A large hydrophobic residue, Phe120 of ZMPDR14, is replaced with a smaller hydrophobic Val in ZMPDR14, and in an offset, the ZMPDR14 uses a large aromatic Tyr83 to substitute His83 of ZmPDR01 on another side of the binding-site. As a consequence, ZMPDR14 and ZM PDR01 have a ligand binding-pocket of equivalent size. A similar variation was also observed in the Arabidopsis proteins, atTFL1/atFT (data not shown). This binding-site variation may be associated with the antagonistic effects of TFL1and FT. Analysis of the geometric and electric property of putative anion binding site in ZmPDR01 (FIG. 5c) was performed. The binding site topology indicates it is able to well accommodate phosphorylated ligand. The Arg and a few His residues (possibly in protonated state upon phosphate group binding) may play a key role for ligand recognition.

### Example 3

### Identification of the CETS (PDR) Gene Family in Maize, Rice, Arabidopsis and Sorghum

The identification of genes for this gene family was focused upon sequences from the following plant species: Zea mays (maize), Oryza sativa (rice), Hordeum vulgare (barley), Sorghum bicolor (sorghum), Triticum aestivum (wheat), Allium cepa (onion), Arabidopsis thaliana, Glycine max (soybean), and Helianthus ssp. (sunflower species). Related sequences were found from all but barley. The identification of genes relied upon searches of available genomic and/or cDNA sequences for these species. The sequence sets searched were both public and private (Dupont/Pioneer proprietary) sequences. A local implementation of NCBI Blast version 2.0 was used for the sequence searching. The initial starting protein sequence queries were the six publicly known Arabidopsis prototypes of this gene family, At_TFL1 (At5g03840), At_CEN (At2g27550), At_BFT (At5g62040), At_FT (At1g65480), At_TSF (At4g20370), and At_MFT (At1g18100). No other members of this gene family were found in Arabidopsis.

For maize the proprietary ESTs, EST assemblies, and genomic sequences, plus the public GSS and EST and other maize NCBI sequences were searched. All potential hits to conserved regions of the gene family were assembled and curated, and additional rounds of searching were done to extend the genomic and/or transcript sequences across the fullest possible coding region, but also across UTR and intron features of the genes. Successive rounds of back searching were done using the nucleotide and translation sequences until an exhaustive account of the maize gene family sequences was obtained. All gene and transcript sequences were curated to identify start and stop codons, intron-exon boundaries, UTRs, and the summary protein translation.

The approach for rice relied chiefly upon searching a combination of mostly public genome assemblies and cDNA contigs, with some proprietary cDNA supplemental information. The main genomic assemblies were the NCBI genomic contigs, but the BGI (Beijing Genomics Institute) dataset was also searched. The public rice sequence annotations were sometimes wrong, and improved ORF and translation determinations were made where needed. The approach for sorghum was similar, but relied upon the recently publicly released Sorghum GSS sequences. The GSS sequences overlapping this gene family were assembled and annotated for ORF and translation products. The barley, wheat, soybean, and Helianthus searches chiefly relied upon a dual search of proprietary ESTs and public cDNAs/ESTs. For onion, there was a large body of ESTs deposited in Genbank. They were retrieved and searched locally as a captive set. Any hits were assembled and annotated.

The resulting gene count from the various species, ignoring the known six from Arabidopsis, is as follows: Zea mays—28, Oryza sativa—21, Sorghum bicolor—24, Triticum aestivum—2, Allium cepa—1, Glycine max—7, Helianthus sp.—3, for a total gene count of 86.

### Example 4

### Phylogenetic Analysis of the Maize PDR Gene Families and their Tissue Specific Expression

In the Arabidopsis genome, there are six genes comprising a CETS family including FT (flowering locus T) and TFL1 (terminal flower 1) (Kardailsky, et al., 1999; Kobayashi, et al., 1999), ATC (Arabidopsis thaliana CENTRORADIALIS homologue) (Mimida, et al., 2001), BFT (Brother of FT and TFL1), MFT (Mother of FT and TFL1), TSF (Twin sister of FT) (Kobayashi, et al., 1999). FT and 1TFL1 genes are the important regulators of flowering time with antagonistic action. The FT is an activator, whereas TFL1 is a repressor of flowering (Kardailsky, et al., 1999; Kobayashi, et al., 1999). Constitutive expression of FT in transgenic Arabidopsis plants causes early flowering, and constitutive expression of TFL1 causes late flowering. Other members of this gene family have been classified by their effect on flowering time. Over expression of MFT and TSF led to early flowering and over expression of ACT led to late flowering. No data are available for BFT (Yoo, et al., 2004). However, the loss-of function mutants of ATC and MFT showed no obvious phenotypes indicating that these two genes rather have a role that is different from regulation of flowering time (Mimida, et al., 2001; Yoo, et al., 2004). No functions were assigned to them.

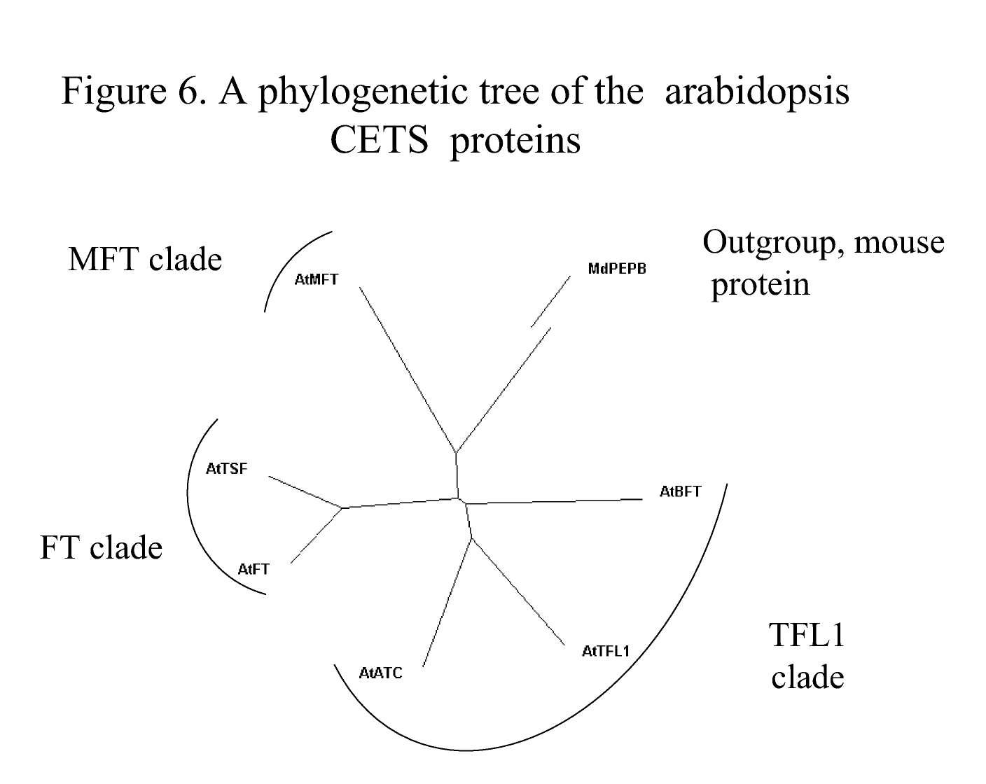

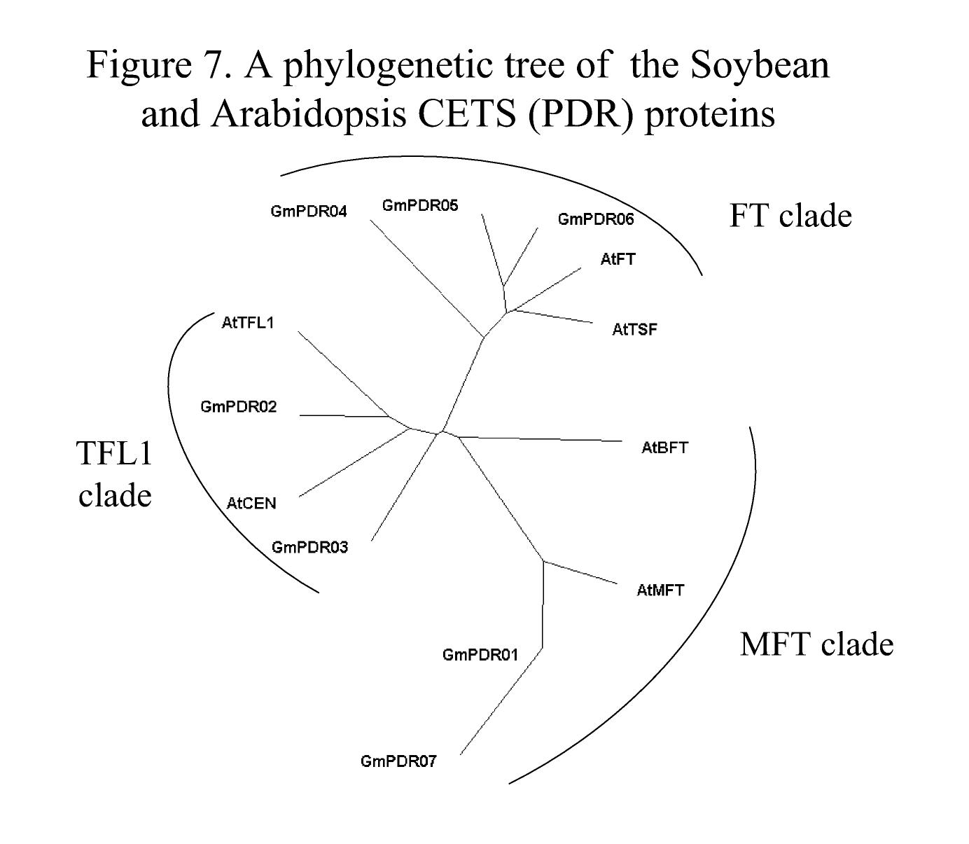

A phylogenetic tree constructed by neighbor-joining method (PAUP program), for Arabidopsis proteins including the mouse PEPB protein as an outgroup, delineated three clades which were named according their founders: the FT clade, the TFL1 clade, and the MFT clade (FIG. 6). Extensive search of the soybean (Glycin max) EST database revealed seven PDR genes. The putative soybean proteins are grouped into the three clades on the phylogenetic tree similar to Arabidopsis (FIG. 7).

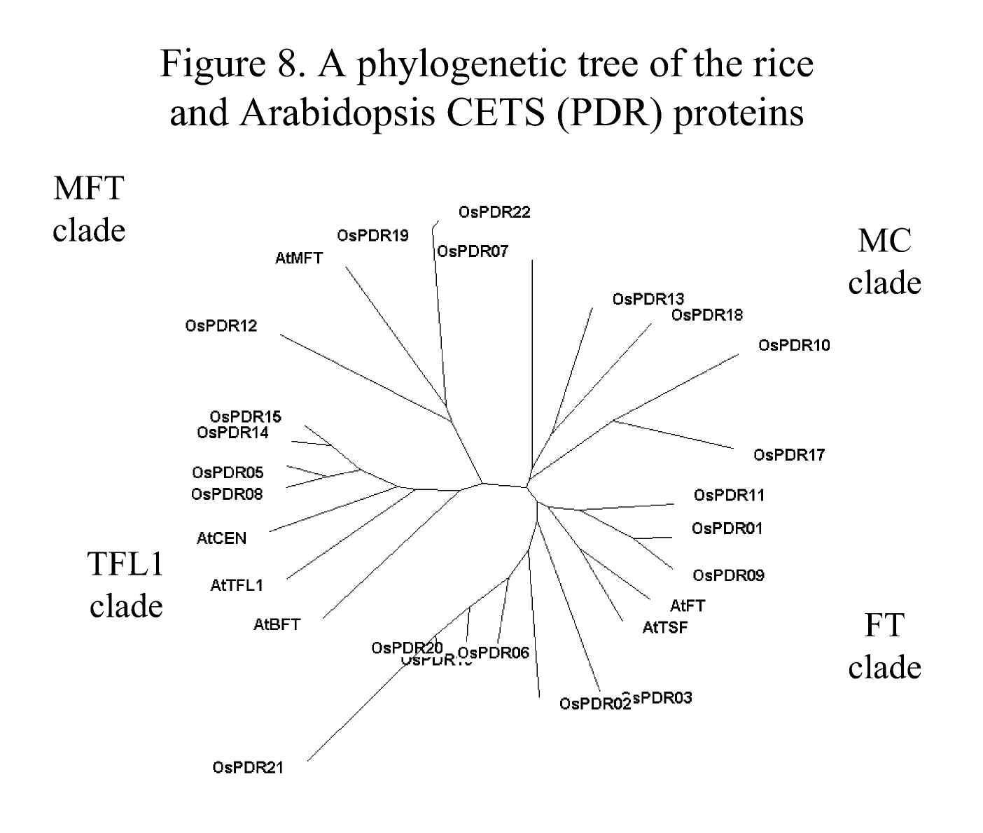

The rice genome contains 22 PDR genes. A phylogenetic tree of rice CETS proteins revealed four clades including three clades described for dicots (FT, TFL1, MST) and a new lade, which was named “the MC (monocot) clade” (FIG. 8). Thus, the PDR gene family is larger and more complex in monocots than in dicots.

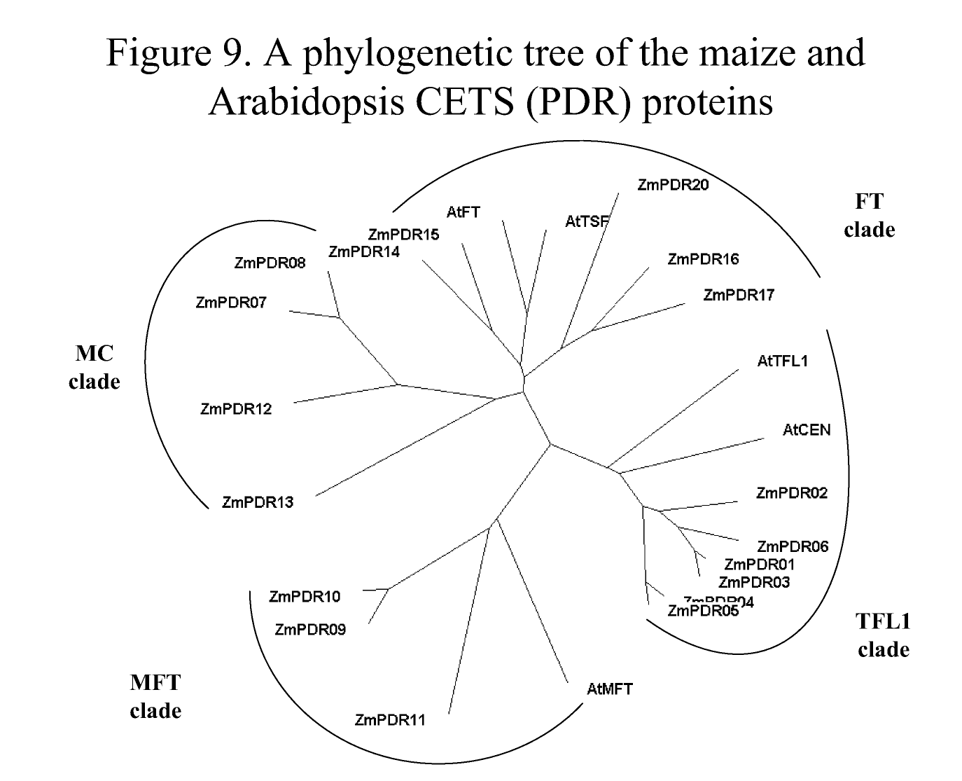

A Pioneer proprietary EST database, public Genomic Survey Sequences (GSS) and Maize assembled genomic sequences (TIGR and ISU-MAGI) were extensively searched, and 33 maize PDR genes were identified. Eighteen of the identified sequences are represented by their corresponding full-length proteins. Partial gene sequences are available for the other members. These 18 complete versions were chosen for the phylogenetic analysis. There are four clades of the CETS proteins in maize, as was the case for rice (FIG. 9). It appears that monocots have the additional clade of the CETS genes (the MC clade) that is not found in dicots.

To predict function of maize PDR genes, we searched the RNA expression profiling MPSS (Massively Parallel Signature Sequencing) (Brenner, et al., 2000), proprietary database that represents more than 200 tissue samples under both normal and stressed conditions. MPSS technology generates 17-mer sequence tags that are unique identifiers of the cDNAs (Brenner et al., 2000). The MPSS expression profiling revealed that genes from different clades showed tissue-specific patterns of expression, suggesting specific functions for each lade.

The maize ‘TFL1’ lade is composed of 6 genes, which are closely related. Coding regions of ZmPDR01, ZmPDR03 and ZmPDR06 shared 85% homology at nucleotide sequence level, while introns shared 55% homology

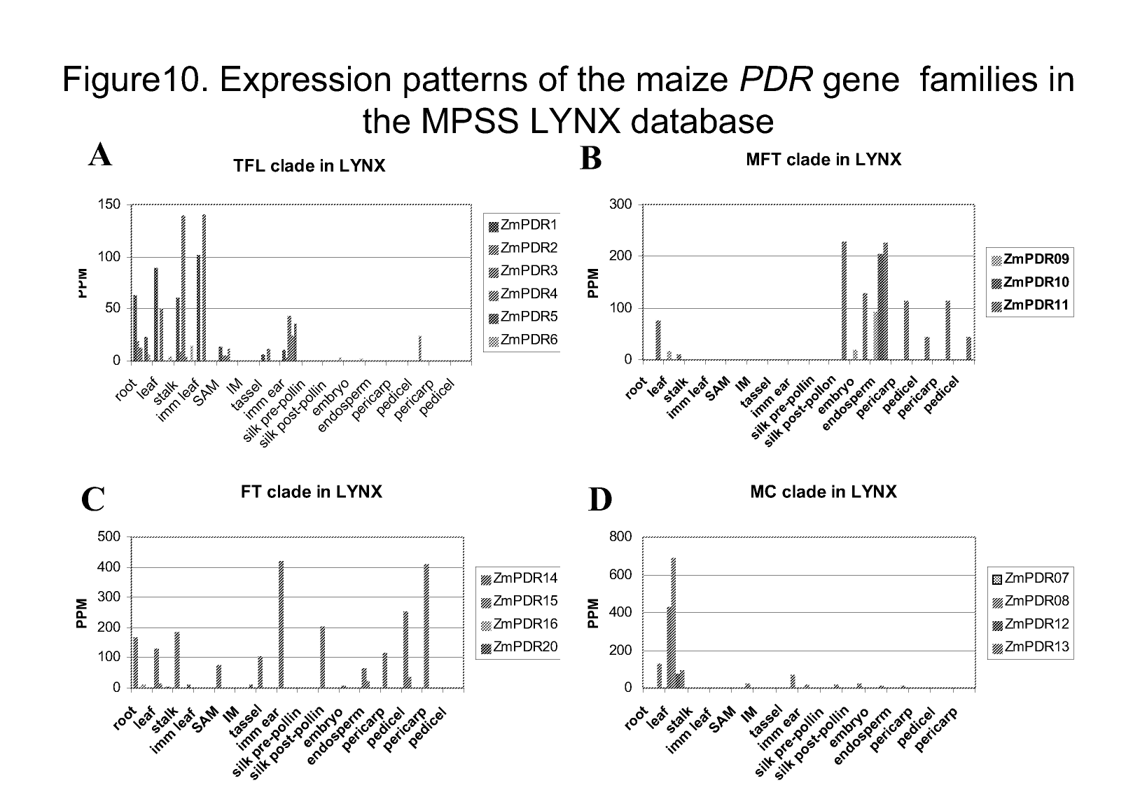

Coding regions of ZmPDR04, and PDR05 shared 75% homology, but the introns showed only 28% homology. ZMPDR01, ZmPDR02, ZmPDR04, ZmPDR05 and ZmPDR06 are mapped to chromosomes 3, 4, 2 10 and 4, respectively. According MPSS profiling, ZmPDR02 gene is expressed at low level in roots, stalks, immature ears, and silk. ZmPDR04/05 showed expression predominantly in reproductive tissues. ZmPDR04 is expressed in tassel, immature ears and pedicel, which is a maternal tissue connecting kernels with cob. ZmPDR05MPSS tags were only in the ear tips (FIG. 10A).

The ‘MFT’ lade is composed of 3 genes ZmPDR09, ZmPDR10, and ZmPDR11, which are expressed mostly in kernels. ZmPDR09 and ZmPDR10 are highly related sharing 94% homology within the coding sequences and 50% homology within introns. ZmPDR09, ZmPDR10 and ZmPDR11 are mapped to chromosomes 8, 3 and 6, respectively. FIG. 10B contains associated expression data. ZmPDR09 is expressed in the embryo and the endosperm. ZmPDR10 is more abundant in the aleurone layer. ZmPDR11 has shown abundant expression in the embryo, endosperm and silk after pollination. A low level of expression may be detected in roots, leaves and tassel. Manipulation of the ZmPDR09, ZmPDR10, and ZmPDR11 genes may result in modification of kernel traits in transgenic plants.

The ‘FT’ clade is composed of 4 genes ZmPDR14, ZmPDR15, ZmPDR16 and ZmPDRC06. According to MPSS profiling, the ZmPDR14 gene is expressed in both vegetative and reproductive tissues (FIG. 10C). MPSS tags for ZmPDR15, ZmPDR16 and ZmFTC06 are detectable at low level in similar tissues. ZmPDR14, ZmPDR15 and ZmPDR16 are mapped to chromosomes 8, 6 and 5, respectively.

The ‘MC’ clade is specific to monocots. It is composed of 4 genes: ZmPDR12, ZmPDR07, ZmPDR13, and ZmPDR08. ZmPDR12 is mapped to chromosome 3. Each of the genes are expressed preferentially in leaves (FIG. 10D). ZmPDR07 and ZmPDR08 are duplicated genes, sharing 85% homology within the coding regions and 63% within introns. Transcription levels of these two genes are responsive to water availability. The level of their expression is 20 times higher in leaves under well-watered conditions than under the drought stress. Manipulation of the ZmPDR07 and ZmPDR08 in transgenics may result in modification of leaf traits and drought tolerance.

### Example 5

### RNA In Situ Hybridization of the Maize PDR Genes from the ‘TFL1’ Phylogenetic Clade

MPSS expression profiling provided the tissue-specific pattern of expression of the PDR genes. Each tissue or organ is composed of many different cell types with specific functions. Gene expression is identified at the cellular level within a target organ with the help of in situ hybridization. The analysis provides the next level of cell-specific expression profiling on cellular level for prediction of possible gene function. Sense and anti-sense RNA were labeled with isotope S³⁵ using T3 or T7 RNA polymerases according to the manufacturer's protocols (Promega). Sense probes were used as controls, which indicated the background level while anti-sense probes produced true hybridization signals.

### ZmPDR01 Gene is Expressed in Vascular Bundles of Developing Leaves and Stem.

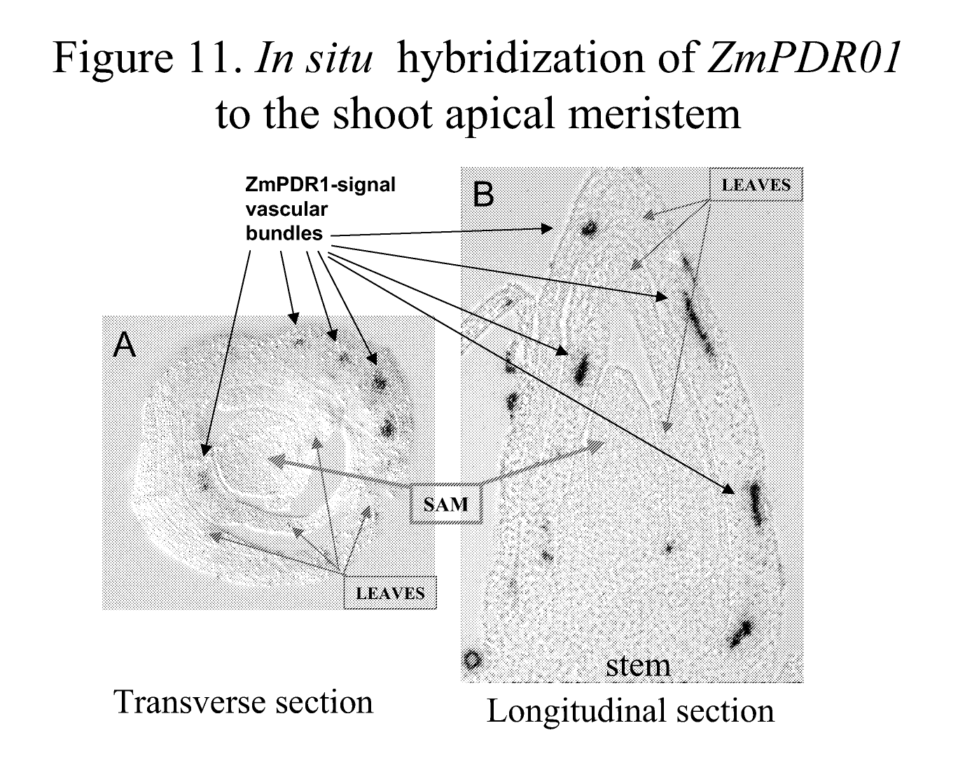

ZmPDR01 anti-sense RNA showed a strong signal in vascular bundles. The hybridization signal was found in primordial provascular cells as well as in the cells which surround mature vascular bundles with differentiated phloem and xylem (FIG. 11). On transverse sections the immature leaves appeared as concentric circles, which are wrapped around SAM (shoot apical meristem) (FIG. 11A). The hybridization signal is concentrated around vascular bundles in a form of isolated islands on the transverse sections (FIG. 11A), while on the longitudinal sections the hybridization signals concentrated in a form of elongated islands around vascular bundles (FIG. 11B). At this stage the leaves are growing in a spiral mode wrapping the SAM.

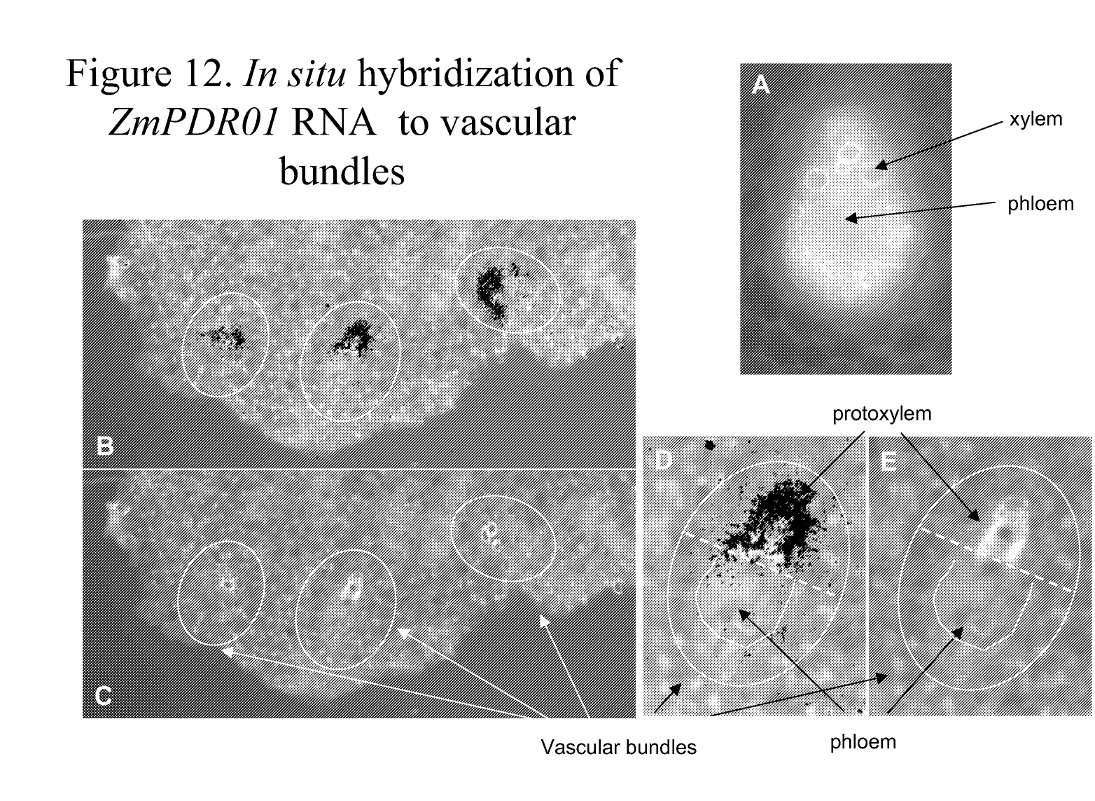

Vascular bundles appear as bright spots under UV illumination due to bright fluorescence of secondary cell walls in xylem (FIG. 12A). Under higher magnification, strong signal can be seen in the primordial vascular bundle cells (in cambial cells) in young leaf (FIG. 12B, C). Hybridization signal can be detected in vascular bundles with well developed xylem vessels, mostly from the adjacent cells which are presumably still in the process of differentiation into xylem, protoxylem and tracheids (FIG. 12D, E). No obvious signal was detected from the phloem and companion cells. These observations show that ZmPDR01 could be involved in the control of provascular cell identity and on later stages for protoxylem cell identity. ZmPDR03 and ZmPDR6 belong to the same sub-branch on the phylogenetic tree as ZmPDR01 (FIG. 9). The genes showed a similar pattern of expression (FIG. 10A) suggesting similarity in function. The expression pattern for ZmPDR01, ZmPDR03 and ZmPDR6 is predicted to be associated with vascular bundles as well.

### ZmPDR02ZmPDR04ZmPDR05 Genes are Expressed in Vascular Bundles of Developing Ears.

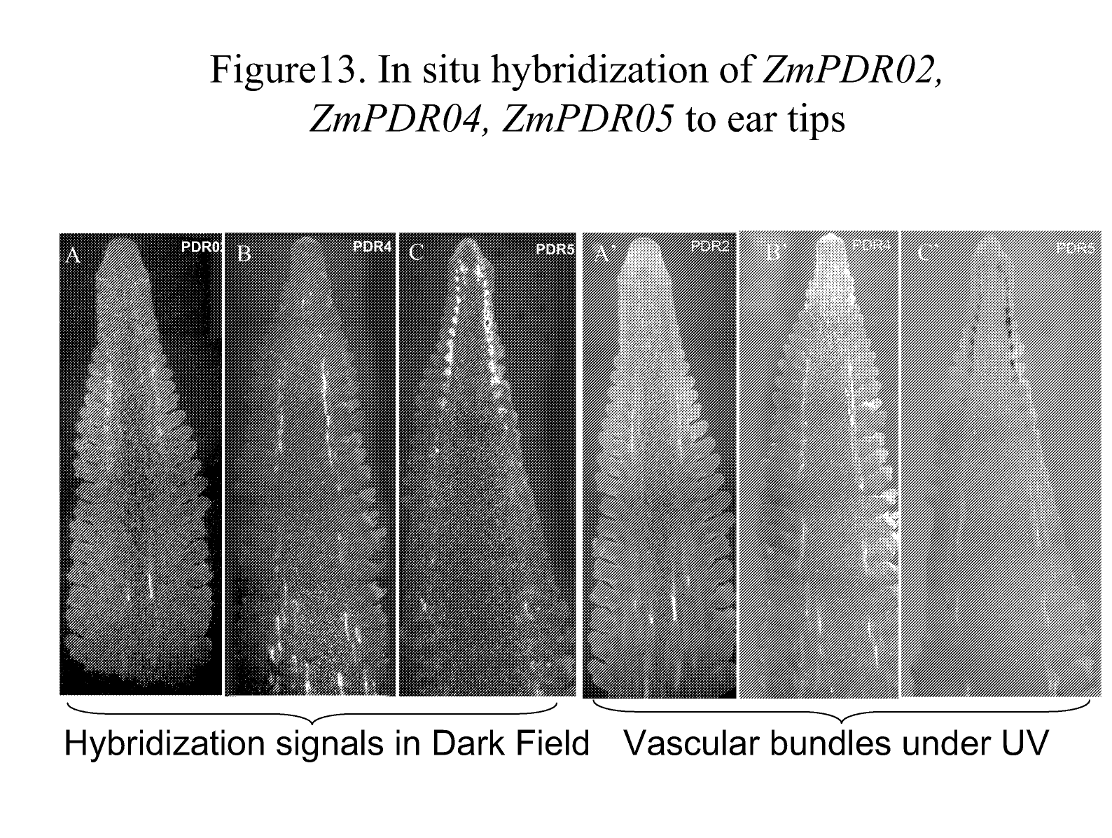

ZmPDR02, ZmPDR04 and ZmPDR05 are members of the sub-group on the TFL1 clade which are expressed in immature ears (FIG. 9, 10). RNA in situ hybridization showed strong signal from the ZmPDR02104/05 anti-sense RNAs in components of vascular bundles on the longitudinal sections of immature ears (FIG. 13) as well as some specific differences. No apparent hybridization signals were found in the upper cell layers of the inflorescence meristem, spikelet pairs, or in spikelet meristems for the ZmPDR02104/05 probes.

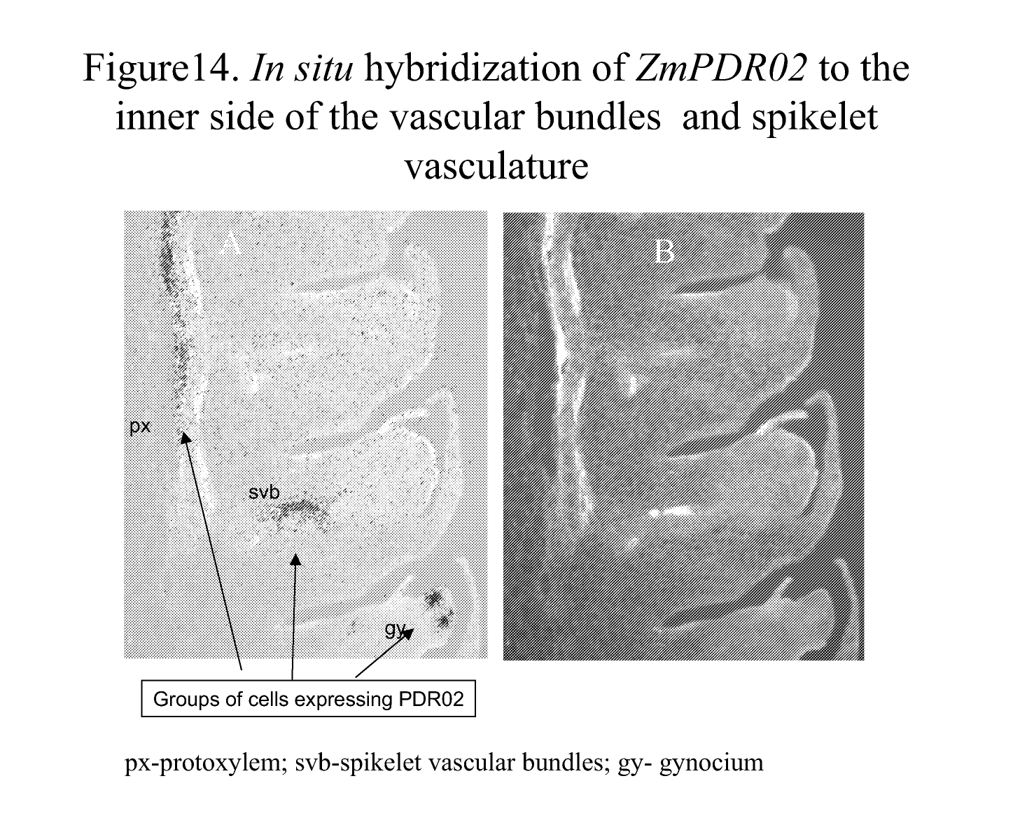

The ZmPDR02 expression initially becomes evident in groups of cells underlying the foundation of each late spikelet pair meristem and each spikelet approximately two to four cell layers within the developing inflorescence stem. These cells have no specific morphological features at that stage except their location (FIG. 13A). The older spikelets below the tenth to twelfth spikelet from the top of the inflorescence have no cells with detectable expression of the ZmPDR02. At the level of 15^{th} to 20^{th} spikelets from the top, expression of ZmPDR02 can be detected within the vascular bundle cells, which are located on inner side of the vascular bundles (FIG. 14A) closer to the axis of the inflorescence stem. These are most likely protoxylem cells. The ZmPDR02 expression can be also seen in vascular bundles within spikelet stem (rachis) (FIG. 14A). In addition the expression of ZmPDR02 can be found in various components of female upper and lower florets (FIG. 13A) such as stamens or at the basement of gynocium. These groups of cells have no specific morphology at that stage of development. ZmPDR02 would be actively transcribed in protovascular cells as well as in proto-xylem or tracheids.ZmPDR04 showed the simplest pattern of expression, which is evident in cells of vascular bundles at the level of SPM (spikelet pair meristems) and below where the vascular bundles develop visible protoxylem and xylem (FIG. 13B).

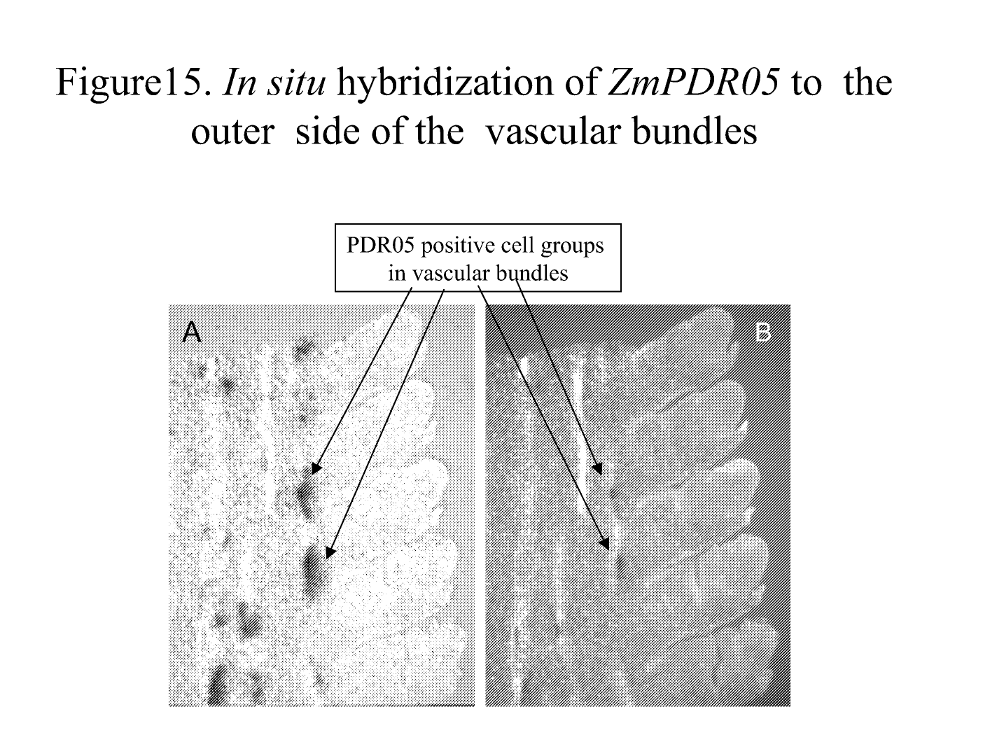

RNA in situ hybridization found strong signal from the ZmPDR05 anti-sense RNA in various groups of cell in the longitudinal sections of the immature ear (FIG. 13C). ZmPDR05 expression is evident in groups of cells underlying inflorescence meristem (FIG. 13C). The primary clusters are small and composed of 2 to 4 labeled cells, which are located inside the inflorescence meristem (IM) below the 6th or 8th layers of cells from the top surface. At the level of primary spikelet pair meristems the groups of labeled cells are located inside the growing ear approximately ¼ of the inflorescence stem diameter from the surface. The distribution of labeled cells has a characteristic segmental pattern with clusters of labeled cells at the base of each spikelet meristem. These cells have no specific morphological features except their location at this stage of development (FIG. 13C). At the lower part of the ear inflorescence ZmPDR05 expressing cells are located on the outer side of vascular bundles, which underly each spikelet (FIG. 15A). This gene is expressed in phloem cells. In some places the expression of ZmPDR05 can be traced to the individual cells apparent from the cross sectioning of the conducting cells. Comparative analysis of ZmPDR02, ZmPDR04 and ZmPDR05 expression in the developing ear showed that each of the three genes is expressed in specific groups of cells within a vascular bundle (FIG. 13, 14, 15). The ZmPDR05 is the first gene of this group to be expressed in progenitor cells produced by inflorescence meristem. The expression of ZmPDR05 as well as ZmPDR02 is induced in rhythmic fashion in small groups of cells if not in individual cells, which are entering the process of differentiation into the spikelet pair meristem. These genes are apparently activated in several other groups of cells in the developing florets. The position of the ZmPDR02 and ZmPDR05 expressing cells coincides with the position of the future bundle vessel plexuses between major vessel bundles and spikelet vessel bundles.

These observations are consistent with various studies that revealed a complex hierarchal organization of ear vascular system. The system is composed of multiple vessel bundles, which are interconnected in multiple plexuses underlying each spikelet. Major vessel bundles are running through the main stem of the cob each of which forms plexuses with the vessel bundles of each spikelet, which are in turn are branched into multiple micro bundles supplying water and nutrients to the developing kernel and various parts of the florets (Cheng, 1995).

### Example 6

### Vascular Bundle Specific Promoters of ZmPDR01, 02, 03, 04, and 05 Genes

The ZmPDR01, ZmPDR02, ZmPDR04, ZmPDR05 genes are expressed within different zones of the vascular bundles as has been shown by in situ hybridization (FIGS. 11-15). These genes are the source of the vascular specific promoters used to target expression in the particular type cells of the vascular bundles.

ZmPDR01 is expressed in vascular bundles of vegetative tissues (leaves and stems) with well developed xylem vessels, especially in the adjacent cells which are presumably still in the process of differentiation into xylem, protoxylem and tracheids. Thus its promoter may be used for gene expression in protoxylem.

ZmPDR02 is actively transcribed in protovascular cells as well as in proto-xylem or tracheids of developing ears. ZmPDR04 is evident in cells of vascular bundles at the level of SPM (spikelet pair meristems) and below where the vascular bundles develop visible protoxylem and xylem. Both promoters may be used for gene expression in the ear specific protoxylem. ZmPDR05 is apparently expressed in phloem cells of the developing ear and its promoter may be used for specific expression in phloem.

### Example 7

### Promoter Optimization for Maize PDR Gene Expression

Manipulation of the expression of different members of the PDR gene family in transgenic maize has been performed. Constitutive expression of ZmPDR01 gene in maize resulted in overall enhanced growth effects in maize plants (see, EXAMPLE 1). In order to create a desirable phenotype in a particular organ/tissue and optimize gene expression in tissue/organ specific manner, the ZmPDR01 gene was linked to a number of tissue/organ-specific promoters. To modify ear traits, ZmPDR01 was linked to the TB1 promoter, which is expressed in axillary branches (Doebley, et al., 1997; Hubbard, et al., 2002), to generate PHP24948. The in situ experiments show that ZmPDR01 is expressed in vascular bundles and could be used to modify stalk traits. The S2A promoter, which is expressed in intervascular cambium around vascular bundles and inside vascular bundles in young stem (Abrahams, et al., 1995) was employed to drive ZmPDR01 in PHP24945. The expression of ZmPDR01 in a root-specific manner was driven with the NAS2 promoter (Mizuno, et al., 2003) in PHP24943. ZmFTM1 and ZmFTM3 promoters, which are predominantly expressed in meristematic tissues, also manipulated the expression of ZmPDR1 in the PHP24951 and PHP24952, respectively, to enhance ear and tassel sizes and avoid late flowering phenotype. The targeting of the tassel traits in transgenic maize, was accomplished using anther and pollen specific promoter SGB6 (U.S. Pat. Nos. 5,470,359 and 5,837,850, Huffman) with both PDR overexpression and RNAi vectors designated as PHP24949 and PHP24950 respectively.

### Example 8

### Application of PDR Gene for Yield Enhancement in Soybeans and Small Grain Crops

High spikelet density induced by over expression of ZmPDR1 gene is particularly valuable for crops with perfect flowers (male and female florets formed on the same spikelets) such as rice, wheat, sorghum, barley, rye that have spikes (head) equivalent to maize tassels. Over expression of PDR genes could increase the spikelet number per spike and grain yield of transgenic plants. The PDR genes ectopically expressed in transgenic soybean plants would be expected to increase the overall biomass, and the number of pods, that leading to higher yielding soybean varieties.

### Example 9

### Enhanced Agronomic Traits in ZmPDR03ZmPDR04 and ZmPDR05 Transgenic Plants

Transgenic plants were produced for ZmPDR03, ZmPDR04 and ZmPDR05 genes that are closely related to ZmPDR01 and ZmPDR02. Corresponding cDNAs were integrated into a transcriptional cassette between the Ubiquitin promoter and PINII terminator in a standard vector for Agrobacterium transformation. Twenty-five events were generated for each construct (PHP23176, UBI::ZmPDR03, PHP25992, UBI::ZmPDR04; and PHP26034 UBI::ZmPDR05)). Greenhouse-raised TO plants exhibited an extended period of vegetative growth, produced more and larger leaves, and thicker stalks. Transgenic plants also produced tassels having an increased spikelet density and an increased amount of pollen. The ZmPDR03/04/05 transgenics had phenotypic expression similar to that of ZmPDR01 and ZmPDR02 transgenic plants (see, Example 1). This indicates that ZmPDR genes from the same phylogenetic clade are able to create similar transgenic traits in plants (see, FIG. 9).

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

What is claimed is:

1. A method for plant characteristics, the method comprising:
a. introducing into a plant cell a recombinant expression cassette comprising a polynucleotide whose expression, alone or in combination with additional polynucleotides, functions as a plant developmental regulator polypeptide within the plant;
b. culturing the plant cell under plant forming conditions to produce a plant; and,
c. inducing expression of the polynucleotide for a time sufficient to alter the architecture of the plant.

2. The method of claim 1 wherein the plant is a monocot.

3. The method of claim 1 wherein the plant is a dicot.

4. The method of claim 1 wherein the plant is maize, barley, wheat, rice, rye, oats, millet, sorghum, soybean, canola or sunflower.

5. The method of claim 2 wherein the plant is maize.

6. The method of claim 1 wherein the polynucleotide is linked to a promoter.

7. The method of claim 1 wherein the polynucleotide is selected from the group consisting of: SEQ ID NOS: 1, 3, 5, 7, 9, 11, 13, 15, 17, 19, 21, 23, 25, 27, 29, 31, 33, 35, 37, 39, 41, 43, 45, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183.

8. A transgenic plant produced by the method of claim 1.

9. A transgenic seed produced by the transgenic plant of claim 8.

10. The method of claim 1, where expression of plant developmental regulator polypeptide within the plant is increased.

11. The method of claim 1, where expression of plant developmental regulatory polypeptide within the plant is decreased.

12. The method of claim 10, where said plant has increased kernel number per ear.

13. The method of claim 10, where said plant has an increased tassel spikelet density.

14. The method of claim 10, wherein said plant has an increased tassel branch number.

15. The method of claim 10, where said plant has increased pollen production.

16. The method of claim 10, where said plant has improved canopy shape.

17. The method of claim 10, where said plant has increased photosynthetic capacity in leaf tissue.

18. The method of claim 10, where said plant has improved stalk strength.

19. The method of claim 10, where said plant has improved plant standability.

20. The method of claim 10, where said plant has altered vascular bundle structure or number.

21. The method of claim 10, where said plant has increased root biomass.

22. The method of claim 10, where said plant has enhanced root growth.

23. The method of claim 10, where said plant has modulated shoot development.

24. The method of claim 10, where said plant has modulated leaf development.

25. The method of claim 11, where said plant has shorter plant internodes.

26. The method of claim 11, where said plant has stunted growth.

27. The method of claim 11, where said plant is a dwarf plant.

28. A method for increasing plant harvestable yield, the method comprising:
a. introducing into a plant cell a recombinant expression cassette comprising a polynucleotide whose expression, alone or in combination with additional polynucleotides, functions as a plant developmental regulator polypeptide within the plant;
b. culturing the plant cell under plant forming conditions to produce a plant; and
c. inducing expression of the polynucleotide for a time sufficient to increase the harvestable yield of the plant.

29. The method of claim 28 wherein the plant is a monocot.

30. The method of claim 28 wherein the plant is a dicot.

31. The method of claim 28 wherein the plant is maize, barley, wheat, rice, rye, oats, millet, sorghum, canola, sunflower and soybean.

32. The method of claim 29 wherein the plant is maize.

33. The method of claim 28 wherein the polynucleotide is linked to a promoter.

34. The method of claim 28 wherein the polynucleotide is selected from the group consisting of: SEQ ID NOS: 1, 3, 5, 7, 9, 11, 13, 15, 17, 19, 21, 23, 25, 27, 29, 31, 33, 35, 37, 39, 41, 43, 45, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183.

35. A transgenic plant of the method of claim 28.

36. A transgenic seed of the transgenic plant of claim 35.

37. An isolated nucleic acid comprising a polynucleotide sequence of the PDR coding region or a complement thereof.

38. An isolated nucleic acid wherein the polynucleotide has at least 75% sequence identity to the PDR coding region as determined by GAP 10 analysis using default parameters over the entire length of the sequence, or a complement thereof, wherein expression of the polynucleotide modulates the level of PDR expression in a plant.

39. An isolated nucleic acid wherein the polynucleotide hybridizes under high stringency conditions to the PDR coding region, or a complement thereof, wherein expression of the polynucleotide modulates the level of PDR expression in a plant.

40. An isolated nucleic acid wherein the polynucleotide comprises the PDR coding region, or a variant thereof, wherein the expression of the variant modulates the level of PDR expression in a plant.

41. An isolated nucleic acid wherein the polynucleotide comprises a fragment of the PDR coding region.

42. The isolated nucleic acid fragment of claim 41 wherein the fragment is a functional fragment.

43. An expression cassette comprising the nucleic acid of claim 37 operably linked to a promoter, wherein the nucleic acid is in sense or antisense orientation.

44. A non-human host cell stably transformed with the expression cassette of claim 43.

45. The host cell of claim 44 that is a plant cell.

46. The host cell of claim 44 that is a bacterial cell.

47. A plant stably transformed with the expression cassette of claim 43.

48. An isolated nucleic acid comprising a polynucleotide sequence of the PDR coding region, or a complement thereof.

49. An isolated polynucleotide selected from the group consisting of:
a. a polynucleotide having at least 80% sequence identity, as determined by the GAP algorithm under default parameters, to the full length sequence of a polynucleotide selected from the group consisting of SEQ ID NOS: 51, 89, 91, 97, 119, 127, 139, 147, 165 and 171; wherein the polynucleotide encodes a polypeptide that has PDR functions; and
b. a polynucleotide encoding a polypeptide selected from the group consisting of SEQ ID NO: 50, 90, 92, 98, 120, 128, 140, 148, 166, and 170; and
c. a polynucleotide selected from the group consisting of SEQ ID NOS: 51, 89, 91, 97, 119, 127, 139, 147, 165 and 171; and
d. a polynucleotide which is complementary to the polynucleotide of (a), (b), or (c).

50. A recombinant expression cassette, comprising the polynucleotide of claim 43, wherein the polynucleotide is operably linked, in sense or anti-sense orientation, to a promoter.

51. A host cell comprising the expression cassette of claim 50.

52. A transgenic plant comprising the recombinant expression cassette of claim 50.

53. The transgenic plant of claim 52, wherein said plant is a monocot.

54. The transgenic plant of claim 52, wherein said plant is a dicot.

55. The transgenic plant of claim 52, wherein said plant is selected from the group consisting of: maize, soybean, sunflower, sorghum, canola, wheat, alfalfa, cotton, rice, barley, millet, peanut and cocoa.

56. A transgenic seed from the transgenic plant of claim 52.

57. A method of increasing yield in plants, comprising:
a. introducing into a plant cell a recombinant expression cassette comprising the polynucleotide of claim operably linked to a promoter; and
b. culturing the plant under plant cell growing conditions; wherein the plant architecture is improved.

58. The method of claim 57, wherein the plant cell is from a plant selected from the group consisting of: maize, soybean, sunflower, sorghum, canola, wheat, alfalfa, cotton, rice, barley, millet, peanut and cocoa.

59. A method of increasing yield in a plant, comprising:
a. introducing into a plant cell a recombinant expression cassette comprising the polynucleotide of claim 49 operably linked to a promoter;
b. culturing the plant cell under plant cell growing conditions; and
c. regenerating a plant form said plant cell; wherein the plant architecture is improved.

60. The method of claim 59, wherein the plant is selected from the group consisting of: maize, soybean, sorghum, canola, wheat, alfalfa, cotton, rice, barley, millet, peanut, and cocoa.
